(19) United States
(12) Patent Application Publication
(10) Pub. No.: US 2025/0283020 A1
(43) Pub. Date: Sep. 11, 2025

(54) MICROPHYSIOLOGICAL SYSTEM AND USES THEREOF

(71) Applicants: INSTITUT NATIONAL DE LA SANTE ET DE LA RECHERCHE MEDICALE, Paris (FR); CENTRE NATIONAL DE LA RECHERCHE SCIENTIFIQUE, Paris (FR); UNIVERSITÉ PAUL SABATIER TOULOUSE III, Toulouse (FR); ECOLE NATIONAL VÉTÉRINAIRE DE TOULOUSE, Toulouse (FR); INSTITUT NATIONAL DE LA RECHERCHE POUR L'AGRICULTURE, L'ALIMENTATION ET L'ENVIRONNEMENT (INRAE), Paris (FR)

(72) Inventors: Dimitri HAMEL, Toulouse (FR); Audrey FERRAND, Pompertuzat (FR); Julie FONCY, Ramonville Saint Agne (FR); Laurent MALAQUIN, Ayguesvives (FR)

(21) Appl. No.: 18/716,343

(22) PCT Filed: Dec. 7, 2022
(86) PCT No.: PCT/EP2022/084852

§ 371 Date: Jun. 4, 2024

(30) Foreign Application Priority Data
Dec. 8, 2021 (EP) 21306727.5

(51) Int. Cl. C12M 3/06; C12M 1/00; C12M 1/12

(52) U.S. Cl. CPC C12M 23/16; C12M 23/22; C12M 23/58; C12M 25/02

(57) ABSTRACT

The invention relates to a microfluidic device (1) comprising a frame (32) and two opposite walls (16a,b), the two opposite walls (16a,b) and the frame (32) delimiting together a chamber (2): —the chamber (2) comprising a first zone (4) and a second zone (5), —the second zone (5) comprising a porous member (3) extending in the chamber (2) and comprising a first surface (9) and a second surface (10) opposite to the first surface (9), the first surface (9) separating the chamber (2) in the first zone (4) and in the second zone (5), —the frame (32) comprising at least a first and a second sets of ports (11, 12, 13, 14), the first set of port comprising at least two ports (11, 12) arranged in the frame (32) for fluid circulation within in the first zone (4) and the second set of ports comprising at least two ports (13, 14) arranged in the frame (32) for fluid circulation within the second zone (5), and—the two ports (13, 14) of the second set of ports being open (i) in a microchannel (15) extending through the porous member (3), or (ii) in a cavity (19) arranged between the second surface (10) of the porous member (3) and the frame (32) of the chamber (2). The invention relates to microphysiological systems comprising a microfluidic device and cultured cells on the top surface of the hydrogel matrix. The microphysiological systems may be used to model biological surface or biological tissue at physiological interface comprising a luminal and a stromal compartments, such as colon, pancreas or skin tissue.

### TECHNICAL FIELD

The invention relates to the field of microfluidic devices usable for producing biological artificial tissue or for ex vivo or in vitro culture of biological tissue(s), e.g., plant or animal tissues. The invention relates to the field of organ on chip and microphysiological systems. The invention relates to the field of in vitro or ex vivo models of living tissues, for example tissues playing the role of interface or barrier between different entities, such as the epithelial, e.g., colon, lung or skin tissue, or endothelial tissues, e.g. lymphatic or blood vessel, using a microphysiological system.

### TECHNICAL BACKGROUND

Microphysiological systems (MPS) are in vitro platforms composed of cells; explants derived from tissues/organs; and/or organoid cell formations of human, animal, or plant origin in a micro-environment that provides and supports biochemical/electrical/mechanical responses to model a set of specific properties that define organ or tissue function. They present an opportunity to bring new tools to biology, medicine, pharmacology, physiology, and toxicology. The International Consortium for Innovation and Quality in Pharmaceutical Development (IQ) defines the MPS as “culture systems that go beyond traditional 2D culture including several of the following design aspects: a multicellular environment within a biopolymer/biomaterial or tissue-derived matrix; a 3D architecture; the inclusion of mechanical cues such as stretch or infusion for intestinal peristalsis, flow rate; control of mass transfer through microfluidic features; the incorporation of primary cells or derived from stem cells; and/or inclusion of stromal components” (Fabre et al., Lab on a Chip, 2020).

Presenting a surface closed to 30 m², the intestinal epithelium is the second main interface, just after the pulmonary epithelium, between the external world and our inner body (Helander et al., Scand J Gastroenterol, 2014. 49(6): 681-689.). This lining of monolayer cells is a dynamic barrier enabling the absorption of dietary nutrients while excluding harmful compounds, and an actor of the immune surveillance by sampling the luminal antigens (Yu et al., J Biomed science, 2018. 25(1)). This ability to both controls the uptake across the mucosa and protect from damage of harmful substances is defined as the intestinal barrier function (IBF) (Yu et al., J Biomed science, 2018. 25(1)). In order to maintain a functional IBF, the different cell populations (stem cells, transit-amplifying progenitors and differentiated cells-enterocytes, enteroendocrine, goblet cells) composing the colon epithelium are entirely renewed over a 5 to 7-day period by the intestinal stem cells (ISC) residing at the bottom of the colon crypt. A very specific environment, the intestinal crypt niche, tightly controls the homeostasis and integrity of the crypt cells. The niche includes the basal lamina (BL), stromal cells that participate in the renewal and differentiation processes by secreting gradients of diverse factors (Wnt, R-Spondin, Noggin, BMP . . . ) along the crypt axis, and the extracellular matrix (ECM) which regulates cell fate via matrix-cell interactions (Onfroy-Roy et al., Cells. 2020; 9(12):2629).

The development of in vitro artificial colonic microdevice, which reproduces more faithfully complex in vivo systems, are important tools to improve our understanding of the human gut physiology and pathologies. A main flaw of currently used flat culture systems in petri dishes is their lack of integration of the tissue topological aspects and their impacts on the tissue behavior and fate. In this context, synthetic in vitro models such as microphysiological systems allow the control of specific parameters including tissue topology, rigidity and nutrients and molecular flow distribution, as well as the 3D architecture and cellular heterogeneity.

3D intestinal organoids allow recreating 3D intestinal epithelial mini-organs to study the ISC capacity to reconstitute a fully polarized and functional epithelium with its diverse cell populations. However, being cultivated in 3D within a MATRIGEL™ matrix, these hollow spherical structures do not properly recapitulate the intestinal/colon topology aspects and make difficult to access the lumen compartment. In consequence, although the 3D human colon organoid model represents an interesting tool to study the proliferation and differentiation processes of the intestinal epithelium, more relevant tools remain needed to investigate other aspects such as the impact of the tissue topology on the cell behavior and fate, the lumen/epithelial interface, the mechanical stimuli or the control of molecular gradients.

The creation of in vitro models mimicking the physical and chemical cues of intestinal tissues has attracted a strong interest during the last 10 years and has provided considerable technological breakthrough in the fields. A specific attention has been taken to develop 3D in vitro systems reproducing the intestinal topology (crypt and/or villi) and an accessible lumen/epithelium interface (Verhulsel et al., Lab Chip. 2021; Creff et al., Biomaterials. 2019; Kim et al., Lab Chip. 2012; Kim et al., Integr Biol (Camb). 2013; Nikolaev et al., Nature. 2020). Several models have reproduced the small intestine crypt/villi topology, one by structuring a Collagen (Coll) I hydrogel allowing the culture of fibroblast within the gel (Verhulsel et al., Lab Chip. 2021) and the other by combining a photopolymerizable hydrogel, which supports the growth of intestinal cell lines, with high resolution stereolithographic 3D printing (Creff et al., Biomaterials. 2019). In both cases the groups have demonstrated the impact of the topology on the establishment of a uniform epithelium, cell polarization and migration. Using a PDMS replication approach, the groups of Magness and Allbritton (Wang et al., Cell Mol Gastroenterol Hepatol. 2017; 5(2):113-130; Wang et al., Biomaterials. 2017; 128:44-55) structured colon crypts topology into a layer of Collagen I hydrogel placed onto the membrane of a TRANSWELL™-type insert. This set-up helps to control the apico-basal compartment of the epithelium and thus to establish a biochemical gradient along the crypt-villus axis. However, none of these models are coupled to a microfluidic system, and thus do not allow to finely tune and monitor the culture environment over time.

To date, only two models are reported coupled to a microfluidic system: The most reported one displays a central chamber separated by a porous membrane that allows epithelial cells 2D culture and to control the apico-basal flow. Two hollow side chambers permit the control of the air pressure and thus the stretching or compression of the central chamber membrane (Kim et al., Lab Chip. 2012; Kim et al., Integr Biol (Camb). 2013). The device has a dual liquid flow circulation but is devoid of a hydrogel matrix comprising a compartment or a printed surface.

A more evolved system has recently been published by Nikolaev et al. They reported a colonic MPS coupled to a fluidic system allowing an active control of the luminal compartment above the colonic epithelium derived from human organoids (Nikolaev et al., Nature. 2020). This model is based on the structuration by laser microdissection of the hydride hydrogel (mixture of Collagen I and MATRIGEL™), reproducing a ‘lumen’ and colonic crypt-like structures. However, the two main flaws of this device are a poorly reproduced colon crypt topology, the design being largely restricted by the device conception itself, and the lack of possible active control of the basal compartment.

Chen et al. (Sci Rep 5, 13708 (2015)) describes a device for the 3D culture of intestinal cells comprising a porous silk fiber matrix comprising a channel, the inner side of which is optionally printed with a pattern (luminal part of the channel) and in which the cells are cultured. However, this device does not allow the circulation of two streams of liquids.

Wang et al. (Cell Mol Gastroenterol Hepatol. 2017; 5(2):113-130) describes a PDMS printing stamp for printing a pattern representing colon crypts on the surface of a hydrogel for cell culture. The cell culture device of TRANSWELL™ type does not allow the circulation of two streams of liquids.

Venzac et al. (Microsystems & Nanoengineering, Springer Nature, 2020, 6 (1)) describes a system of movable walls for microfluidic devices acting as valves controlling the flow of a liquid through the channels of the devices. There is no mention of a hydrogel matrix comprising a compartment.

Yamada et al. (Lab on a Chip, 2016, 16 (11), pp. 2059-2068) describes a microchip for neuron culture and axon growth, the preparation of which includes microprinting a pattern on the cell culture surface by means of a print stamp. A micro-channel separates the growing area into two zones. The device is not configured to allow the circulation of two streams of liquids.

US 2018/0200714 A1 describes a microfluidic device comprising a PDMS or hydrogel matrix comprising a woven thread and one or more channels. However, there is no mention of liquid circulation on surface of the matrix or a printed surface of the matrix.

Therefore, there is a need for a new microfluidic device or microphysiological system suitable for mimicking the different compartments of a biological tissue, such as for example luminal and stromal compartments, or different tissues or organs.

There is a need for a new microfluidic device or microphysiological system suitable for mimicking the physiological and/or pathological rigidities, mechanical stresses and changes of composition of biological fluids (e.g., gas composition, fluid composition, etc.) of the luminal and stromal compartments of a biological tissue or organ.

There is a need for a new microfluidic device or microphysiological system suitable for mimicking the physiological and/or pathological cell-cell interactions, cell-matrix interactions, or cell-microbiota interactions of different compartments of a biological tissue, or organ, or different tissues or organs.

There is a need for development of new in vitro culture models allowing to more closely reproducing tissue or organ physiological and pathological conditions by controlling multiple parameters of the cellular microenvironment (e.g., matrix, rigidity, flow management . . . ).

There is a need for a new device suitable for increasing the speed, efficiency, and safety of pharmaceutical development and testing, which can adequately recapitulate the dynamics of drug-organ/tissue, drug-drug, and drug-organ/tissue-organ/tissue interactions in humans for different biological organs/tissues, such as in gut tissues, i.e., gastric, intestine or colon tissues, skin or lung tissues.

There is a need for a new microfluidic device or microphysiological system suitable for controlled studies of the dynamics of metabolism and/or signaling within and between human tissues or organs, or for controlled studies of microbiome or of disease within and between human organs/tissues, such as gut tissue, i.e., gastric, intestine or colon tissues, skin or lung tissues.

There is a need for a new microfluidic device or microphysiological system able to be easily manufactured in large numbers.

There is a need for a new microfluidic device or microphysiological system able to be easily connected in parallel or in series and to mimic a succession or an organization of different organs or biological tissues.

There is a need for a new microfluidic device or microphysiological system able to recapitulate cell-matrix, cell-ceII, cell-tissue, and cell-organism interactions.

There is a need for a new microfluidic device or microphysiological system able to ensure reproducibility and stiffness control of a porous member, such as a hydrogel matrix, to allow cell adhesion and proliferation.

There is a need for a new microfluidic device or microphysiological system able to mimic luminal and stromal biological compartments.

There is a need for a new microfluidic device or microphysiological system able to mimic stretch or infusion for intestinal respiration or peristalsis.

There is a need for a new microfluidic device or microphysiological system able to mimic a tissue topology, such as a colon.

There is a need for a new microfluidic device or microphysiological system suitable for high resolution and in-depth optical imaging of the cultured cells or modelled tissue or biological surface.

There is a need for a new microfluidic device or microphysiological system suitable for bioprinting.

There is a need for a new microfluidic device or microphysiological system suitable for omics analysis, i.e., configured for allowing injection of materials, and for allowing recovery of suitable amounts of samples.

There is a need for a new microfluidic device or microphysiological system allowing production of epithelium or endothelium with increased total surface.

There is a need for a new microfluidic device or microphysiological system compatible with different biomaterials or materials.

There is a need for a new microfluidic device or microphysiological system allowing a customizable number of inlet/outlet ports to control spatial and temporal heterogeneity.

The present invention has for purpose to satisfy all or part of those needs.

### SUMMARY

According to one of its objects, the present disclosure relates to a microfluidic device (1) comprising two opposite walls (16a,b) and a frame (32) defining a chamber (2) comprising at least a first porous member (3) and at least a first and a second zones (4, 5):
the frame (32) comprising at least a first and a second sets of ports (11, 12, 13, 14), the first and second sets of ports (11, 12, 13, 14) comprising at least two ports arranged in the frame for fluid circulation within, respectively, the first and the second zone (4,5),
the first porous member (3) extending partly in the chamber (2) and comprising a first surface (9) and a second surface (10) opposite to the first surface (9), the first surface (9) defining at least partly an interface between the first zone (4) and the second zone (5) of the chamber (2),
the first zone (4) being defined in the chamber (2) at least being between the first set of ports (11, 12) and the first surface (9) of the first porous member (3), and
the second zone (5) comprising the first porous member (3) and
   a microchannel (15) configured in the first porous member (3), the microchannel (15) extending between the ports (13, 14) of the second set of ports, and/or
a portion of the chamber (2) defined at least between the second set of ports (13, 14) and the second surface (10) of the porous member (3).

The second zone (5) may be delimited by a part of the frame (32).

According to another of its objects, the present disclosure relates to a microfluidic device (1) comprising a frame (32) and two opposite walls (16a,b), the two opposite walls (16a,b) and the frame (32) delimiting together a chamber (2):
the chamber (2) comprising a first zone (4) and a second zone (5),
   the second zone (5) comprising a porous member (3) extending in the chamber (2) and comprising a first surface (9) and a second surface (10) opposite to the first surface (9), the first surface (9) separating the chamber (2) in the first zone (4) and in the second zone (5),
the frame (32) comprising at least a first and a second sets of ports (11, 12, 13, 14), the first set of port comprising at least two ports (11, 12) arranged in the frame (32) for fluid circulation within in the first zone (4) and the second set of ports comprising at least two ports (13, 14) arranged in the frame (32) for fluid circulation within the second zone (5), and the two ports (13, 14) of the second set of ports being open
(i) in a microchannel (15) extending through the porous member (3), or
(ii) in a cavity (19) arranged between the second surface (10) of the porous member (3) and the frame (32) of the chamber (2).

Herein, “first porous member” and “porous member” are used interchangeably, except if the context dictates otherwise.

The frame (32) and the opposite walls (16a,b) delimit together a chamber (2) arranged to be able to be filled with a liquid.

Within the disclosure, the expression “microfluidic device” intends to refer to a device configured for transporting small volumes of fluids at controlled flow rates. A “small” amount may range from a nanoliter to a milliliter. A microfluidic device typically comprises at least a microfluidic channel plus all of the relevant micro-features plus inlet/outlet ports that make it work.

Within the disclosure, the expression “two opposite walls” intends to refer to two structures, each defining a surface, positioned opposite one to the other, parallel, or not. The walls may be planned or curved, and of any shape such as round, rectangular, square, or trapezoidal.

Within the disclosure, the term “frame” intends to refer to a structure supporting or holding the opposite walls and defining with the opposite walls a chamber.

The frame may have any form, such as round, triangular, square.

Within the disclosure, the term “chamber” intends to refer to internal volume defined by the two opposite walls and the frame, and configured for receiving and holding various materials, such as fluids, porous member, cells, etc.

Within the disclosure of the invention, the term “zone” intends to refer to a region of the internal volume of the chamber, this region having functional and/or physical delimitations.

With the disclosure, the expression “porous member” intends to refer to an element configured for allowing passage and diffusion of various materials, such as fluids, gas, biological materials or cells, from the porous member to the at least first and/or second zone or from the at least first and/or second zone to the porous member. A porous member may be configured to be porous from start or to become porous or have an increase of porosity over time. A change from non-porous to porous material or in terms of increase of porosity may be obtained with partially degradable materials, such as gelatin, collagen, matrigel, or hyaluronic acid.

The porosity of a material is the volumetric fraction of pores in the material. These pores can be located on its surface and/or in its internal structure. Porosity is associated with the density of the material, and with the nature of its compounds and the existence of empty spaces between them.

Porosity of a porous material may be measured by any known technics in the art, such as Mercury porosimetry, Helium pycnometry, or else FIB-SEM.

The porosity of a porous material, expressed as fraction of the volume of voids over the total volume of the porous member, may range from about 10% (low porosity) to about 90% (high porosity).

A porous material may have pores having an average pore size ranging from about 50 nm to about 500 μm, for example from about 100 nm to about 350 μm. The average pore size may be measured by any known methods in the art.

As example of suitable method, one may mention the scanning electronic microscopy (SEM) as described in Louis et al. (Biotechnol. Bioeng., 2017, 114: 1813-1824).

Within the disclosure, the term “microchannel” intends to refer any elongated space, tube, duct, pipe, conduit, along which a fluid substance can be transported. A channel may be delimited, at least along part of its length, by walls having an inner face that define an interior space. A microchannel may have at least a portion of its inner wall being a porous surface allowing passage of fluid from the microchannel to the porous member and/or from the porous member to the microchannel.

Within the disclosure the term “port” intends to refer to a space or gap, of any form, allowing passage or access from the interior, or internal volume, to the exterior of the chamber, and reciprocally.

As shown in the Examples section, the inventors have developed a microfluidic device and a microphysiological system (MPS) suitable for mimicking a human colon-like model, recapitulating in vitro more closely the 3D human colonic epithelium microenvironment including its topology and matrix support stiffness. The Examples show that the newly developed microfluidic device and microphysiological system can advantageously be used to mimic and model cell tissues, organ-like structures, or biological surfaces. For example, the newly developed microfluidic device and a microphysiological system may be used to model a biological surface, such as an endothelium or an epithelium, and study the interactions and reactions of the biological surface with the stromal and/or luminal environments.

The configuration of the newly developed microfluidic devise and microphysiological system comprising at least a first and a second zones and a porous member, the first zone being above the porous member and the second zone being a microchannel in the porous member or being below the porous member allows reproducing a biological surface at the interface of the first and second zone. For example, the stromal and luminal compartments of a biological tissue, for example a colonic tissue, may be reproduced by the first and second zones. The first and second zones are in fluid communication with the exterior of the chamber by means of set of ports allowing the circulation and control of various fluids inside the first and second zones, mimicking, for example, the fluids coming from the stromal and/or luminal compartments. For example, in case of an epithelium, such as a colonic tissue, the circulation of a fluid in the second zone may mimic a blood circulation, and the circulation of a fluid in the first zone may mimic the circulation of an alimentary bolus. A biological surface which can be modelled with the newly developed microfluidic devise and microphysiological system may be, for example, a gut tissue (gastric, intestine, colon, or rectal tissue), a skin tissue, a lung tissue, an organ canal (e.g., a hepatic, a gland or a pancreatic canal).

As shown in the Examples, the MPS integrates a microfluidic device that allows active control of both the first zone, for example representing an apical/luminal environment, and of the second zone, for example representing a basal/stromal environment, by accurate injection but also sample recovery, as well as in situ imaging of tissue culture. This novel device, combining 3D scaffold and microfluidic addressing, allows a better understanding of the tissue (mechanobiology, architecture, functions . . . ) and is a useful tool to study microbiota, pathogens, and nutrients impacts on the epithelium, e.g., the colon epithelium, of the modelled biological tissue, as well as drug screening.

Advantageously, a microfluidic device and microphysiological system disclosed herein may be coupled to a microfluidic system. The coupled fluidic system makes possible to access and control the ‘luminal’ and/or ‘stromal’ compartments, and to create a gradient of factors which are particularly relevant in the context of the compartmentalized biological surface via the supply.

According to further advantages, the microfluidic device and microphysiological system disclosed herein allows providing control of i) the topology of a biological tissue (e.g., a colon tissue), ii) the rigidity of the supporting scaffold material and iii) the mass transport mechanism by an active dynamic microfluidic control of both the lumen and basal compartments.

According to further advantages, the microfluidic device and microphysiological system disclosed herein may be prepared with the molding of a porous member, such as a polyacrylamide hydrogel, to mimic human biological tissue surface, such as colon crypt topology. The molding may be performed directly within the microfluidic device that may comprises several inlet and outlet ports to ensure continuous perfusion of medium during the culture. The device may comprise two lateral glass windows that provide unique capabilities for a real time imaging of the tissue formation and development during culture.

According to further advantages the microfluidic device, microphysiological system, and movable closure disclosed herein may be easily manufactured with a 3D printing method.

According to another of its objects, the disclosure relates to a microfluidic device (1) comprising two opposite walls (16a,b) and a frame (32) defining a chamber (2) comprising at least a first and a second zones (4, 5):
the frame (32) comprising a movable closure (33) and at least a first and a second sets of ports (11, 12, 13, 14), the first and second sets of ports comprising at least two ports arranged in the frame (32) for fluid circulation within, respectively, the first and the second zone (4, 5),
the movable closure (33) being configured to extend at least partly within the chamber (2) through the first zone (4), the movable closure (33) comprising an outward face (34) and an inward face (35), the inward face (35) being configured to extend at the interface of the first and second zones (4, 5). The inward face (35) may comprise at least partly an imprinting face (36).

A microfluidic device (1) comprising a frame (32) and two opposite walls (16a,b), the two opposite walls (16a,b) and the frame (32) delimiting together a chamber (2):
the chamber (2) comprising a first zone (4) and a second zone (5),
the frame (32) comprising a movable closure (33) and at least a first and a second sets of ports (11, 12, 13, 14),
the first set of ports comprising at least two ports arranged (11, 12) in the frame (32) for fluid circulation within the first zone (4) and the second set of ports comprising at least two ports (13, 14) arranged in the frame (32) for fluid circulation within the second zone (5), and
   the movable closure (33) comprising an outward face (34) and an inward face (35), the inward face (35) comprising an imprinting face (36), and the movable closure (33) extending within the chamber (2) through the first zone (4) up to an interface between the first zone (4) and the second zone (5).

The first zone (4) is filled with the movable closure (33). The interface between the first and second zones (4, 5) is delimited by the imprinting face (36) of the inward face (35).

In some embodiments, the inward face (35) may comprise at least partly an imprinting face (36). The inward face (35) may be configured to be an imprinting face (36). The inward face (35) may be configured to be contacted with the first surface (9) of a porous member (3).

The imprinting face (36) may comprise a plurality of reliefs (27).

The imprinting face (36) may comprise regularly spaced reliefs (27). The regularly spaced relief may be cylindrical reliefs. The cylindrical reliefs may be comprised of cylindrical having a height of about 400 μm, a diameter of about 50-100 μm, and being spaced from each other of about 400 μm. The imprinting face (35) may define protruding structures defining an inverted pattern intended to be imprinted or embossed on the first (9) or second surface (10) of the porous member (3). In some embodiments, the pattern is intended to be imprinted or embossed on the first surface (9) of the porous member (3).

In some embodiments, the chamber (2) may comprise at least a first porous member (3), the first porous member (3) extending partly in the chamber (2) and comprising a first surface (9) and a second surface (10) opposite to first surface (9), the first surface (9) defining at least partly an interface between the first zone (4) and the second zone (5) of the chamber (2).

In some embodiments, the second zone (5) of the chamber (2) may comprise:
a porous member extending in the chamber (2) and comprising a first surface (9) and a second surface (10) opposite to first surface, the first surface (9) delimiting the interface between the first zone (4) and the second zone (5) of the chamber (2), and the two ports (13, 14) of the second set of ports being open
(i) in a microchannel (15) extending through the porous member (3), or
(ii) in a cavity (19) arranged between the second surface (10) of the porous member (3) and the frame (32) of the chamber (2).

In some embodiments, the inward face (35) of the movable closure (33) is in closed contact with the first surface (9) of the first porous member (3). In some other embodiments, the inward face (9) of the movable closure (33) is in closed contact with the second surface (10) of the first porous member (33). The movable closure (33) may be a top and/or a bottom closure (17, 8).

In some embodiments, the frame (32) may comprise at least two lateral walls (6, 7) arranged between the opposite walls (16a,b).

In some embodiments, the frame (32) may comprise a top closure (17) and a bottom closure (8) and at least one of the top and bottom closure (17, 8) is a movable closure.

In some embodiments, the frame (32) may comprise a bottom closure (8) being a fixed closure integral with the lateral walls (6, 7).

The lateral walls (6, 7) may be connected together by a top (17) or a bottom (8) closure, and for example are connected together by the bottom closure (8).

In some embodiments, at least one of the bottom and of the top closures (17, 8) comprises at least one port (18a,b).

In some embodiments, at least one of the bottom and of the top closures (17, 8) may comprise at least one protruding element (29, 30, 31) intended to exert a mechanical constraint or pressure on the porous member (3).

In some embodiments, one or both of the opposite walls (16a,b) is or are made, in whole or in part, of a material transparent to at least one wavelength in the range from infrared to UV wavelengths.

In some embodiments, the microfluidic device (1) as disclosed herein may comprise least an additional microchannel (15a) in the first porous member (3) and/or a cavity (19) defined between the second surface (10) of the first porous member (9) and the frame (32) of the chamber (2), and the frame (32) comprising optionally at least an additional set of ports comprising at least two ports (20, 21) arranged in the frame (32) for fluid circulation within said additional microchannel (15a) and/or said cavity (19).

In some embodiments, the microfluidic device (1) as disclosed herein may be such that
the second zone (5) comprises the porous member (3) comprising a microchannel (15a) and an additional microchannel (15b), or
the second zone (5) comprises (a) the porous member (3) comprising the microchannel (15) and (b) the cavity (19),
and the frame (32) comprises an additional set of ports comprising at least two ports (20, 21) arranged in the frame (32) for fluid circulation within the second zone (5) and being open in the additional microchannel (15a) or in the cavity (19).

In some embodiments, the chamber (2) may comprise a second porous member.

In some embodiments, the first porous member (3), and optionally the at least second porous member, may be positioned between, and in contact with, the opposite walls (16a,b) and the lateral walls (6, 7).

In some embodiments, the first porous member (3), and optionally the at least second porous member, are a porous matrix. A porous matrix may be hydrogel matrix.

Within the disclosure, the expression “hydrogel matrix” intends to refer water-swollen polymeric materials that maintain a distinct three-dimensional structure. Hydrogels may be broadly classified into two categories:
(i) permanent or chemical gel, such as PEG diacrylate, polyacrylamide, gelatin-methacrylate, or hyaluronic acid-methacrylate. They are called “permanent” or “chemical” gels since they are covalently cross-linked (i.e., hydrogen bonds replaced with stronger and stable covalent bonds) networks. They attain an equilibrium swelling state which depends on the polymer-water interaction parameter and the crosslink density. In physically cross-linked gels, dissolution is prevented by physical interactions, which exist between different polymer chains (Hennink & Nostrum, 2002).
(ii) reversible or physical gel, such as collagen, gelatin, or hyaluronic acid. They are called “reversible” or “physical” gels since the networks are held together by molecular entanglements, and/or secondary forces including ionic, hydrogen bonding or hydrophobic interactions. All of these interactions are reversible and can be disrupted by changes in physical conditions or application of stress (Rosiak & Yoshii, 1999).

The porous member (3) may be a monolayer or a multilayer member. For example, a multilayered porous member may comprise several, at least two, distinct layers of different type of materials, such as polymers, or of the same material, such as polymer at different concentrations.

In some embodiments, the first surface (9) of the first porous member (3) may be a cell culturing surface.

In some embodiments, the cell culturing surface may comprise reliefs (25). The reliefs may form a regular pattern. The reliefs may be microcavities or protruding micro-reliefs. The reliefs may be regularly spaced to each other on the cell culturing surface.

In some embodiments, reliefs (25) may be present within the porous member (3).

In some embodiments, with a multilayer porous member, reliefs may be present in a volume within the porous member, at an interface of two layers.

In some embodiments, with a multilayer porous member, reliefs may be present across at least one, two, or more layers.

In some embodiments, the first zone (4) is between the first surface (9) of the first porous member (3) and a top closure (17).

In some embodiments, the second zone (5) is between the first surface (9) of the first porous member (3) and a bottom closure (8).

In some embodiments, at least one of the ports (11, 12, 13, 14, 20, 21) arranged in the frame (32) or in the top or bottom closure (18a,b,c) may be connected to a microfluidic tube.

In some embodiments, at least one port of the at least a first and/or a second sets of ports may be connected to a microfluidic tube (22a,b, 23a,b, 24a,b).

In some embodiments, at least two ports of the at least a first and/or a second sets of ports (11, 12, 13, 14, 20, 21) may be each connected to a microfluidic tube (22a,b, 23a,b).

In some embodiments, at least one port (18a, b, c) the top and/or bottom closure (8, 17) may be connected to a microfluidic tube (24a,b).

In some embodiments, at least one port (11, 12, 13, 14, 20, 21) of the at least a first and/or a second sets of ports may be connected to a sensor or an actuator.

In some embodiments, at least two ports (11, 12, 13, 14, 20, 21) of the at least a first and/or a second sets of ports may be each connected to a microfluidic tube (22a,b, 23a,b) and at least one additional port (11, 12, 13, 14, 20, 21) of the at least a first and/or a second sets of ports and/or at least one port the top and/or bottom closure may be connected to a sensor or an actuator.

Within the disclosure, a “sensor” intends to refer a device that can detect the changes in a physical environment. It can convert physical parameters such as temperature, heat, motion, humidity, pressure, etc. into electrical signals. It can transform this signal into a human readable display and send them through a network for further processing.

Within the disclosure, an “actuator” intends to refer to a device that converts a control signal into a physical change/stimulation in the environment. It obtains a control signal in the form of electric voltage, current, hydraulic fluid, pneumatic or hydraulic pressure. The actuator converts the received control signal into mechanical motion.

In one of its objects, the present disclosure relates to a microphysiological system comprising at least a microfluidic device (1) as disclosed herein and at least one cell type cultured in suitable conditions in the first and/or second zone (4,5) of said microfluidic device (1).

Within the disclosure the expression “microphysiological system” or “(MPS)” intends to refer to two- or three-dimensional cellular constructs in a micro-environment that provides and supports biochemical/electrical/mechanical responses to model a set of specific properties that define organ or tissue function. MPS may also be referred to as “organs-on-chips” or “in vitro organ constructs”. The cellular constructs may be made with various type of cells, such as immortalized cell lines, primary cells from animals or humans, embryonic stem cells, or induced pluripotent stem cells (iPSCs). Organ-on-Chips are microfluidic devices used for mimicking a biological environment, such as human body environment, and which are lined with living cells, such as human cells, and configured to imitate the physiological and mechanical conditions experienced in the biological environment, such as in the human body. They may be used for drug development, disease modeling, and personalized medicine. Microphysiological systems (MPS) are complex, multi-cellular in vitro systems that commonly include three-dimensional aspects, fluid flow, changing pressure or stretch, and multi-organ interactions. These systems are being developed to better mimic some aspects of specific organ systems or combinations of organ systems.

In some embodiments, the microphysiological system may comprise at least one cell type cultured in suitable conditions in suspension in the first and/or in the second zones (4,5).

In some embodiments, the at least one cell type may be cultured in suitable conditions on the first surface (9) of the first porous member (3).

In some embodiments, the at least one cell type cultured in suitable conditions on the inner face of the microchannel (15) extending within the porous member (3).

In some embodiments, the at least one cell type may be cultured in suitable conditions within the porous member (3).

Cultured cells may be adherent or non-adherent cells. In some embodiments, cultured cells are adherent cells.

In one of its objects, the present disclosure relates to an assembly comprising at least two microfluidic devices (1) as disclosed herein or at least two microphysiological system as disclosed herein, the at least two microfluidic devices (1) or at least two microphysiological system being connected in series or in parallel.

For example, in a configuration in series, the first zone (a) of a first microfluidic device (a), or a first MPS (a), may be connected to the first zone (b) of a second microfluidic device (b), or a second MPS (b), by means of a microfluidic tube connecting a first port (a), in fluid communication with the first zone (a), and a first port (b), in fluid communication with the first zone (b). Also, or alternatively, the second zone (a) of a first microfluidic device (a), or a first MPS (a), may be connected to the second zone (b) of a second microfluidic device (b), or a second MPS (b), by means of a microfluidic tube connecting a second port (a), in fluid communication with the second zone (a), and a second port (b), in fluid communication with the second zone (b). The configuration may be repeated at least once, twice, three times, or more.

For example, in a configuration in parallel, a first microfluidic tube comprising at least two branches, e.g., a Y-microfluidic tube, may be connected by a first branch (a) to a first port (a), in fluid communication with a first zone (a) of a first microfluidic device (a), or a first MPS (a), and by a second branch (b) to a first port (b), in fluid communication with a first zone (b) of a second microfluidic device (b), or a second MPS (b), A second microfluidic tube comprising at least two branches, e.g., a Y-microfluidic tube, may be connected by a first branch (a) to a second port (a), in fluid communication with the first zone (a) of the first microfluidic device (a), or a first MPS (a), and by a second branch (b) to a second port (b), in fluid communication with the first zone (b) of a second microfluidic device (b), or a second MPS (b). The first and second ports (a) and the first and second ports (b) are configured for a fluid circulation in the first zone (a) and in the first zone (b), respectively. By multiplying the branches of the first and second microfluidic tubes it may be possible to add further microfluidic device or MPS. Also, or alternatively, the configuration may be repeated with the second zones.

In some embodiments, at least one of the ports of a microfluidic device of a microphysiological system as disclosed herein is in fluid communication with a reservoir.

Within the disclosure, the term “reservoir” intends to refer to a volume that can contain fluids. Reservoirs involve at least one fluidic connection means allowing to put them in fluidic connection with at least one compartment of the microfluidic device, continuously or at some specific times during the operation of the device. A reservoir may contain any fluid, liquid or gas, suitable for feeding a microfluidic device, or a MPS, as disclosed herein. Alternatively, a reservoir may be used to collect any fluid exiting the microfluidic device, or the MPS. A liquid may be or contain, for example, a cell culture media, a buffer, a nutrients solution, or an active pharmaceutical ingredient (API). A reservoir may comprise or be added with a pump for feeding a fluid into the microfluidic device, or the MPS, or for extracting a fluid from the microfluidic device, or the MPS.

In one of its objects, the present disclosure relates to a method for manufacturing a microfluidic device (1) as disclosed herein. The method comprises at least the steps of:
providing a chamber (2) comprising two opposite walls (16a,b) and a frame (32), the frame (32) comprising at least a first and a second sets of ports (11, 12, 13, 14), each of the first and second sets of ports comprising at least two ports arranged in the frame (32) for fluid circulation within the chamber (2),
forming at least a first porous member (3) in the chamber (3), for obtaining at least a first and a second zones (4, 5), the first porous member (3) comprising a first surface (9) defining at least partly an interface between the first and second zones (4, 5) and a second surface opposite (10) to the first surface (9),
the first zone (4) being defined in the chamber (2) at least being between the first set of ports (11, 12) and the first surface (9) of the first porous member (3), and
the second zone (5) comprising the first porous member (3) and
a microchannel (15) in the first porous member (3), the microchannel (15) extending between the ports of the second set of ports (13, 14), and/or
a portion of the chamber (2) defined at least between the second set of ports (13, 14) and the second surface (10) of the porous member (2).

In one of its objects, the present disclosure relates to a method for manufacturing a microfluidic device (1) as disclosed herein, the method comprising at least the steps of:
providing a chamber (2) delimited by a frame (32) and two opposite walls (16a,b),
forming a porous member (3) in the chamber (2), the porous member (3) comprising a first surface (9) and a second surface (10), the first surface (9) separating the chamber (2) in a first and a second zones (4,5), wherein
the frame (32) comprises at least a first and a second sets of ports (11, 12, 13, 14), the first set of ports (11, 12) comprising at least two ports arranged in the frame (32) for fluid circulation within the first zone (4) and the second set of ports comprising at least two ports (13, 14) arranged in the frame (32) for fluid circulation within the second zone (5), and the two ports (13, 14) of the second set of ports being open
(i) a microchannel (15) extending through the porous member (3), or
(ii) in a cavity (19) arranged between the second surface (10) of the porous member (3) and the frame (32) of the chamber (2).

A cavity (19) may be obtained by forming a porous member (3) maintaining an empty space in a part of the second zone (5) of the chamber (2), this part being positioned between the second surface (10) of this porous member and the frame (32). To obtain such empty space, one may use a bottom closure (8) with an inward face protruding inside the chamber (2), within a part of the second zone (5), at the time of forming of the porous member (3) inside the chamber (2). Once the porous member (3) is formed, the bottom closure with an inward face protruding inside the chamber (2) is replaced with a bottom closure (8) with an inward face not protruding inside the chamber (2) or protruding with a less extent compared to the bottom closure with a protruding inward face, so that an empty space is obtained between the second surface (10) of the porous member and the frame (32).

A microchannel (15) in the first porous member (3) may be obtained by:
positioning within the chamber (2) at least one elongated member extending through the ports of the second set of ports (13, 14), and
casting in the chamber (2) at least one material suitable to embed said elongated member and to form a porous member (3).

A microchannel (15) in the first porous member (3) may be obtained by:
positioning within the chamber (2) at least one elongated member extending through the ports of the second set of ports (13, 14), and
   casting in the chamber (2) at least one material suitable to embed said elongated member and to form a porous member (3) and a microchannel (15) extending through said first porous member (3).

Further to the casting of the material suitable to embed the elongated member, the material may undergo a step of curing, polymerization, or hardening to form a porous member.

After obtaining the porous member (3), the elongated member may be removed to obtain a microchannel (15) in the porous member (3). The elongated member may have any size and shape provided that it can be extended through ports of the second set of ports (13, 14) and may be withdrawn after obtaining the porous member (3).

For example, an elongated member may be a tube, a wire, a thread.

In some embodiments, a porous member (3) may comprise a network of microchannels. A network of microchannels may be obtained either by positioning an elongated member with multiple branches or multiple elongated members.

In some embodiments, an elongated member with multiple branches may be a sugar-based elongated member which may be dissolved in water after obtaining the porous member (3).

In some embodiments, the porous member (3) may be a hydrogel matrix.

The step of providing the chamber (2) may comprise a step of bonding two opposite walls (16a,b) to a frame (32), as disclose herein.

In some embodiments, a manufacturing method may further comprise a step of connecting, at least, a first microfluidic tube (22a,b) to a first port (11, 12) and a second microfluidic tube (23a,b) to a second port (13, 14), the first and second ports being from the at least first or second sets of ports and being arranged in the frame (32) for fluid circulation within the first or the second zone (4,5).

In some embodiments, the manufacturing method may further comprise a step of forming reliefs (25) on the first surface (9) of the porous member (3) by contacting the first surface (9) with a movable closure (33) comprising an outward face (34) and an inward face (35), the inward face (35) being an imprinting face (36) configured to extend and contact with the first surface (9) of the porous member (3) and comprising a plurality of reliefs (27).

In some embodiments, the manufacturing method may further comprise a step of forming reliefs (25) on the first surface (9) of the porous member (3) by contacting the first surface (9) with a movable closure (33) comprising an outward face (34) and an inward face (35), the inward face comprising an imprinting face (36) for imprinting reliefs (25) on the first surface (9), and the movable closure (33) being arranged to extend within the chamber (2) through the first zone (4) up to the interface with the second zone (5) to contact with the first surface (9) of the porous member (3).

In some embodiments, the manufacturing method may further comprise a step of forming reliefs (25) on the first surface (9) of the porous member (3) by contacting the first surface (9) with a movable closure (33) comprising an outward face (34) and an inward face (35), the inward face comprising an imprinting face (36) comprising a plurality of reliefs (27), and the movable closure (33) being arranged to extend within the chamber (2) through the first zone (4) up to the interface with the second zone (5) to contact with the first surface (9) of the porous member (3).

In some embodiments, reliefs (25) may be provided within a volume of the porous member.

In one of its objects, the present disclosure relates to a method for manufacturing of a microphysiological system (MPS) as disclosed herein. The method comprises the steps of manufacturing a microfluidic device (1) as disclosed herein and further a step of culturing, in suitable conditions, cells in one zone (4, 5).

In some embodiments, the cells may be cultured on the first surface (9) of the porous member (3). In other embodiments, the cells may be cultured on the inner face of the microchannel (15) configured in the porous member (3).

In one of its objects, the present disclosure relates to a use of a microfluidic device (1) or an MPS as disclosed herein, for cell culture.

In one of its objects, the present disclosure relates to a use of a microfluidic device (1) or an MPS as disclosed herein, for modelling a biological surface.

In one of its objects, the present disclosure relates to a microphysiological system for modelling a biological tissue of a physiological interface, for example a tissue colon, the MPS comprising:
at least one microfluidic device (1) as disclosed herein,
wherein
the first zone (4) contains a cell culture medium in fluid communication with at least two ports of the at least first set of ports (11, 12),
the microchannel (15) contains a physiologically acceptable medium in fluid communication with at least two ports of the at least second set of ports (13, 14), and
at least one cell-type is cultured on the first surface (9) of the porous member (3).

In one of its objects, the present disclosure relates to a method for culturing isolated cells, modelling a cell tissue, or modelling a biological surface, for example a colon tissue or a skin tissue, the method comprising at least the steps of:
providing at least one microfluidic device (1) as disclosed herein,
culturing, in suitable conditions, at least one cell type in at least one of at least first and/or second zones (4, 5), the first and/or second zones (4, 5) containing a cell culture medium in circulation through at least a set of at least two ports from the at least first set and/or second set of ports (11, 12, 13, 14).

In some embodiments, the cells may be cultured on the first surface (9) of the porous member (3).

In some embodiments, a first flux of a first cell culture medium is generated in the first zone (4) by circulating said first medium through at least a first set of at least two ports (11, 12) and/or a second flux of a second cell culture medium is generated in the second zone (5) by circulating said second medium through at least a second set of at least two ports (13, 14), the at least two ports of the first and/or second set of ports (11, 12, 13, 14) being configured for fluid circulation in the first and/or second zone (4, 5).

In one of its objects, the present disclosure relates to a movable closure (33), suitable for a microfluidic device (1) as disclosed herein, the movable closure (33) being configured to extend at least partly within the chamber (2) through the first zone (4), the movable closure (33) comprising an outward face (34) and an inward face (35), the inward face (35) being configured to extend at the interface of the first and second zones (4, 5).

In one of its objects, the present disclosure relates to a movable closure (33), suitable for a microfluidic device (1) as disclosed herein, the movable closure (33) comprising an outward face (34) and an inward face (35), the inward face (35) comprising an imprinting face (36), and the movable closure (33) being arranged for be able to extend within the chamber (2) through a first zone (4) up to an interface with a second zone (5).

The first zone (4) is filled with the movable closure (33). The interface between the first and second zones (4, 5) is delimited by the imprinting face (36) of the inward face (35).

In some embodiments, the inward face (35) may comprise at least partly an imprinting face (36). The inward face (35) may be configured to be an imprinting face (36). The inward face (35) may be configured to be contacted with the first surface (9) of a porous member (33). The imprinting face (36) may comprise a plurality of reliefs (27).

The imprinting face (36) may comprise a plurality of reliefs (27).

The imprinted face (36) may comprise regularly spaced reliefs. The regularly spaced relief may be cylindrical reliefs. The cylindrical reliefs may be comprised of cylindrical having a height of about 400 μm, a diameter of about 50-100 μm, and being spaced from each other of about 400 μm. The imprinted face (36) may define protruding structures defining an inverted pattern intended to be imprinted or embossed on the first or second surface of the porous member. In some embodiments, the pattern is intended to be imprinted or embossed on the first surface of the porous member.

In some embodiment, the inward face (35) of the closure (33) may be surface treated. A treatment of surface may be selected to advantageously to reduce adherence or friction between the inward face (35) of the closure (33) and the first face (9) of the porous member (3). A surface treatment may be selected to allow imprinting of the reliefs in the first surface (9) of the porous member (3) and prevent adhesion of the first surface (9) to the inward face (35) so as to avoid a pullout of the first surface (9) of the porous member (3) when the closure (33) is withdrawn.

In one of its objects, the present disclosure relates to a manufacturing method of a movable closure (33) as disclosed herein. The method may comprise a step of obtaining the closure (33) by molding, 3D printing, or laser dissection.

The method may comprise a step of a surface treatment of the inward face. The surface treatment may be carried out with any agent know in the art for reducing or preventing adhesion between two surfaces, for favoring electrostatic repulsion, or increasing hydrophobicity.

The foregoing and other objects, features and advantages of the invention will be described in more detail by referring to the attached drawings, the following description, and the Examples provided hereafter.

### DESCRIPTION OF THE FIGURES

FIG. 1 illustrates the fabrication workflow of an Microphysiological System (MPS) of the disclosure. FIG. 1A: a 3D printed, U-shaped frame (FIG. 1A1), is assembled with two lateral glass slides and segments of tubes (L, B, L′, B′) (FIG. 1A2). Capillaries are inserted to define the basal/stromal (B,B′) microchanel. FIG. 1B: The MPS chamber was further filled with a PA hydrogel preparation to cover the capillary and inlet/outlet ports (B,B′) (FIG. 1B1). A 3D printed mold comprising an array of 400 μm high and 100 μm diameter pillars arranged into a hexagonal lattice (400 μm period) (FIG. 11B2) is then inserted into the chamber and contacted with the surface of the PA hydrogel preparation in order to imprint and replicate crypt-like structures at the top surface of the PA hydrogel (FIG. 11B1). The PA hydrogel preparation is then reticulated. FIG. 1C: after reticulation of the PA hydrogel, the capillary and the mold were removed from the MPS, and a 3D printed cap was inserted in order to seal the luminal compartment of the MPS.

FIG. 2. FIGS. 2A and 2B represent the confocal imaging of crypt-like structures obtained after imprinting soft PA and hard PA hydrogels with a mold bearing an array of 400 μm high and 100 μm diameter pillars arranged into a hexagonal lattice (400 μm period). Fluorescein-methacrylate was used to stain the PA hydrogel network. Cross-cut reconstructions of the structure profiles were obtained from in plane image acquisition (scale bar: 300 μm). FIG. 2C represents the evaluation of the mean±SD structure diameter, height and mean distance between crypt-like structures obtained from confocal acquisitions.

FIG. 3 represents confocal imaging of crypt-like structures replication in Soft PA (FIG. 3A), and Hard PA (FIG. 3B and FIG. 3C) hydrogels functionalized with Collagen I at 1 or 5 mg/mL (scale bar: 300 μm). Fluorescence intensities obtained for the PA hydrogel network (fluorescein-methacrylate) and Collagen I (AlexaFluor 647 NHS) were monitored (FIG. 3D). Homogeneous interpenetration of PA hydrogel and Coll I was obtained for Soft PA-Coll 1 mg/mL and Hard PA—Coll I 5 mg/mL. FIG. 3E: Analysis of the structure dimensions in the three different configurations revealed that combination of Hard PA hydrogel with a Coll I coating at 5 mg/mL was providing a strong replication accuracy.

FIG. 4 shows an epithelial cell culture in an MPS of the disclosure. Representative brightfield images of Caco-2 cells plated for 24 hours into the MPS containing either soft (FIG. 4A) or hard (FIG. 4B) PA gel imprinted with crypt-like structure and coated with homogeneous collagen 1. Global images (FIG. 4A1 and 4B1, 10X objective, scale bar: 90 μm) and focus on bottom (FIG. 4A2 and 4B2) and top crypts (FIG. 4A3, 4A4, 4B3, and 4B4, 20X objective, scale bar: 40 μm) are represented.

FIG. 5: is a schematic representation in perspective of a microfluidic device (1) according to the disclosure.

FIG. 6 is a schematic representation of a microfluidic device (1) according to the disclosure comprising a movable closure (33) for imprinting. The opposite walls (16a,b) are omitted.

FIGS. 7A and 7B are schematic representations of a microfluidic device (1) according to the disclosure comprising a movable top closure (17) and a fixed bottom closure (8) (FIG. 7A) or movable top and bottom closures (FIG. 7B). The opposite walls (16a,b) are omitted.

FIG. 8 represents a microfluidic device (1) with an additional microchannel (15b) or with a cavity (19). FIG. 8A represents a microfluidic device with an additional microchannel (15b) in the porous member. FIG. 8B represents a microfluidic device with a cavity (19) delimited by the second surface (10) of the porous member (3) and part of the frame (32) comprising the second set of at least two ports (13, 14). The opposite walls (16a,b) are omitted.

FIG. 9 is a schematic representation of a microfluidic device (1) according to the disclosure with a movable top closure (17) and the reliefs (25). The opposite walls (16a,b) are omitted.

FIG. 10 is a schematic representation of a microfluidic device (1) according to the disclosure with a movable top closure (17) and fixed bottom closure (8), and with the ports (11, 12, 13, 14, 18a,b) connected with tubes (22a,b, 23a,b, and 24a,b).

FIGS. 11, 12 and 13 are a schematic representation of a microfluidic device according to the disclosure with top closure (17) bearing protruding movable or actionable elements (29, 30, 31). The thick black arrows illustrate the direction of movements of the actionable elements (29,30,31). The opposite walls (16a,b) are omitted.

FIG. 14 is a schematic representation of an assembly of microfluidic devices according to the disclosure in series. White triangles illustrate the sense of fluid circulation in the zones (4,5) through the ports (11, 12) and (13, 14). The zone (4) mimics a luminal compartment through which is going a luminal flow. The zone (5) mimics a stromal compartment through which is going a stromal flow. By circulating specific and control media in the luminal and stromal compartments it is possible to modulate the inputs. By collecting the media exiting the luminal and stromal compartments, it is possible to measure specific outputs.

FIG. 15 is a schematic representation of an assembly of microfluidic devices according to the disclosure in parallel. White triangles illustrate the sense of fluid circulation in the zones (4,5) through the ports (11, 12) and (13, 14). The zone (4) mimics a luminal compartment through which is going a luminal flow. The zone (5) mimics a stromal compartment through which is going a stromal flow. By circulating specific and control media in the luminal and stromal compartments it is possible to modulate the inputs. By collecting the media exiting the luminal and stromal compartments, it is possible to measure specific outputs. The dotte black arrow illustrates the global sense of fluid circulation in the assembly.

FIG. 16 is a schematic representation of the manufacture of a microfluidic device (1) according to the disclosure.

FIG. 17: FIG. 17A represents a picture of a microfluidic device (1) as disclosed herein. FIG. 17B is picture of crypt-like structures imprinted on the first surface (9) of a porous member (3).

FIG. 18: illustrates a FITC-dextran diffusion assay performed with soft and hard polyacrylamide (PA) hydrogels. FIG. 18A illustrates images of FITC-dextran (3 kDa and kDa) diffusion assay performed within the MPS comprising either a polyacrylamide (PA) hydrogel of soft or hard formulation (depending on Young's modulus wish) between two given times (t1, t2). Acquisition carried out with a wide field microscope (Apotome, Zeiss) with a 5× objective. Scale bar: 500 μm. FIG. 18B illustrates extraction of fluorescence intensity curves along the axis of the FITC-dextran migration front in the PA hydrogel at t1 and t2. FIG. 18C shows a table resuming results from determination of the diffusion coefficients (μm²/sec) of FITC-dextran 3 kDa and 40 kDa in PA hydrogels (soft and hard formulation) from the intensity decay curves measured in FIG. 18A and determined in FIG. 18B.

FIGS. 19A, B and C: illustrate the concept of using a magnetic field gradient for exerting an attractive force on a magnetized bead (paramagnetic, ferromagnetic, super-paramagnetic). The magnet is oriented at 45° so that one of the vertices is closest to the ball (max gradient conditions). The force is proportional to the volume of the baII, the gradient, the magnetization (M=XB, X=magnetic susceptibility) of the ball in the field B induced by the magnet.

### DETAILED DESCRIPTION

### Definitions

Unless otherwise defined herein, scientific and technical terms used in connection with the present invention shall have the meanings that are commonly understood by those of ordinary skill in the art. For example, the Concise Dictionary of Biomedicine and Molecular Biology, Juo, Pei-Show, 2^{nd} ed., 2002, CRC Press; The Dictionary of Cell and Molecular Biology, 3^{rd} ed., 1999, Academic Press; and the Oxford Dictionary Of Biochemistry And Molecular Biology, Revised, 2000, Oxford University Press, may provide one of skill with a general dictionary of many of the terms used in this disclosure. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Reactions and purification techniques are performed according to manufacturer's specifications, as commonly accomplished in the art or as described herein. Further, unless otherwise required by context, singular terms shall include pluralities and plural terms shall include the singular.

Units, prefixes, and symbols are denoted in their Système International des Unités (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic characteristic(s) of the disclosure. It is understood that the different embodiments of the disclosure using the term “comprising” or equivalent cover the embodiments where this term is replaced with “comprising only”, “consisting of” or “consisting essentially of”.

It is understood that wherever aspects are described herein with the language “comprising,” otherwise analogous aspects described in terms of “consisting of” and/or “consisting essentially of” are also provided.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Within the disclosure, the terms “significantly” or “substantially” used to qualify a difference or a change, for example ‘significantly different of” or “substantially different from”, with respect to a feature or a parameter intends to mean that the observe change or difference is noticeable and/or it has a statistic meaning. Conversely, the terms significantly” or “substantially” used to qualify a similitude or an identity, for example “not significantly different from” or “substantially identical to”, with respect to a feature or a parameter intends to mean that any observed change or difference is such that the nature and function of the concerned parameter or feature is not materially affected.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

All publications and other references mentioned herein are incorporated by reference in their entirety. Although a number of documents are cited herein, this citation does not constitute an admission that any of these documents forms part of the common general knowledge in the art.

### Microfluidic Device and Microphysiological System

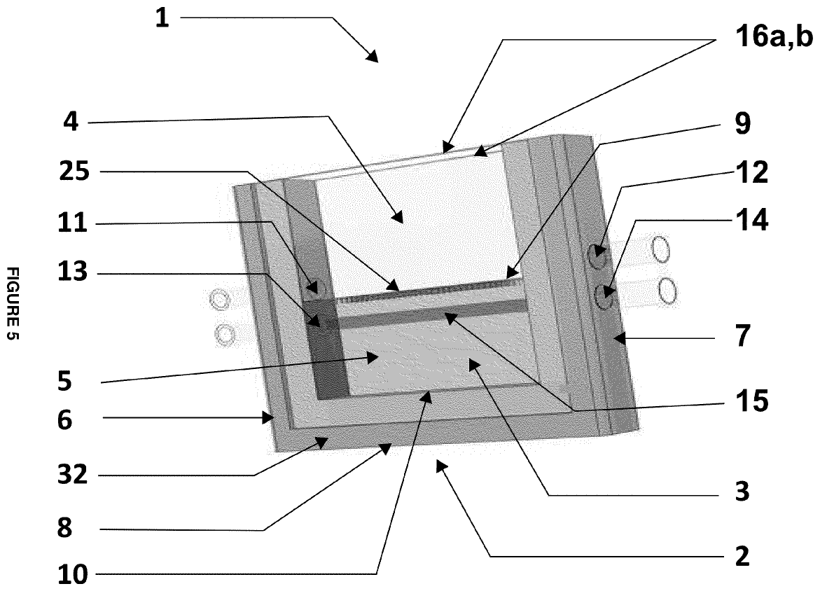

As shown on FIG. 5 or FIG. 10, a microfluidic device (1) as disclosed herein may comprise two opposite walls (16a,b) and a frame (32) defining a chamber (2) comprising at least a first porous member (3) and at least a first (4) and a second (5) zones:
the frame (32) comprising at least a first (11, 12) and a second (13, 14) sets of ports, the first and second sets of ports comprising at least two ports arranged in the frame for fluid circulation within, respectively, the first (4) and the second (5) zone,
   the first porous member (3) extending partly in the chamber (2) and comprising a first surface (9) and a second surface (10) opposite to the first surface, the first surface defining at least partly an interface between the first zone (4) and the second zone (5) of the chamber (2),
the first zone (4) being delimited in the chamber (2) at least being between the first set of ports (11, 12) and the first surface (9) of the first porous member (3), and
the second zone (5) comprising the first porous member (3) and
a microchannel (15) in the first porous member (3), the microchannel (15) extending between the ports (13, 14) of the second set of ports, and/or
a portion of the chamber (2) delimited at least between the second set of ports (13, 14) and the second surface (10) of the porous member (3) (see FIG. 5).

The second zone (5) may be delimited by a part of the frame (32). The second zone may be a region of the chamber delimited by the first surface (9) of the porous member (3) and the part of the frame (32) comprising the at least second set of at least two ports (13, 14).

The first zone (4) may be a region of the chamber (2) delimited by the first surface (9) of the porous member (3) and the part of the frame (32) comprising the at least first set of at least two ports (11, 12).

As shown on FIG. 5 or FIG. 10, a microfluidic device (1) as disclosed herein may comprise a frame (32) and two opposite walls (16a,b), the two opposite walls (16a,b) and the frame (32) delimiting together a chamber (2):
the chamber (2) comprising a first zone (4) and a second zone (5),
   the second zone (5) comprising a porous member (3) extending in the chamber (2) and comprising a first surface (9) and a second surface (10) opposite to the first surface (9), the first surface (9) separating the chamber (2) in the first zone (4) and in the second zone (5),
the frame (32) comprising at least a first and a second sets of ports (11, 12, 13, 14), the first set of port comprising at least two ports (11, 12) arranged in the frame (32) for fluid circulation within in the first zone (4) and the second set of ports comprising at least two ports (13, 14) arranged in the frame (32) for fluid circulation within the second zone (5), and the two ports (13, 14) of the second set of ports being open
(i) in a microchannel (15) extending through the porous member (3), or
(ii) in a cavity (19) arranged between the second surface (10) of the porous member (3) and the frame (32) of the chamber (2).

The frame (32) and the two opposite walls (16a,b) may be formed of a single piece delimiting a chamber (2).

As shown on FIG. 10, the frame (32) and the two opposite walls (16a,b) may be three parts assembled together to delimit a chamber (2). The two opposite walls (16a,b) are in tight contact with the frame (32).

The frame (32) comprises two lateral walls (6, 7). The frame (32) comprise a top part where can be positioned a top closure (17) and a bottom part where can be poisoned a bottom closure (8).

As shown on FIG. 10, the chamber (2) is delimited laterally by the frame (32) and the two opposite walls (16a,b). The frame (32) delimits the sides of the chamber (2). The two opposite walls (16a,b) delimit the front and back of the chamber. The chamber (2) comprises a top part and a bottom part. The first zone (4) and the second zone (5) are delimited laterally by the frame (32) and the two opposite walls (16a,b). The first zone (4) is in the top part. The second zone (5) is in the bottom part. The first zone (4) is above the second zone (5). The chamber (2) comprises a top opening. The chamber may comprise a top closure (17) and a bottom closure (8). The top closure (17) may be movable. The bottom closure (8) may be fixed. The bottom closure (8) may be integral part of the frame (32).

The chamber (2) is delimited laterally by the two lateral walls (6, 7) of the frame (32). The two lateral walls (6, 7) of the frame (32) delimits the sides of the chamber (2).

The first surface (9) is delimiting an interface between the first zone (4) and the second zone (5).

The frame (32) comprises a first set ports comprising two ports (11, 12), or more, open in fluid communication in the first zone (4). The frame (32) comprises a second set ports comprising two ports (11, 12), or more, open in fluid communication in the first zone (5).

As shown on FIG. 10, the porous member (3) is contained, or positioned, in the second zone (5) with its first surface (9) delimiting an interface between the first zone (4) and the second zone (5). The porous member (3) is laterally in contact with the frame (32) and the opposite walls (6, 7). The first surface (9) is facing the top of the chamber (2).

The second surface (10) is facing the bottom of the chamber (2).

The porous member (3) may fill completely the second zone (5).

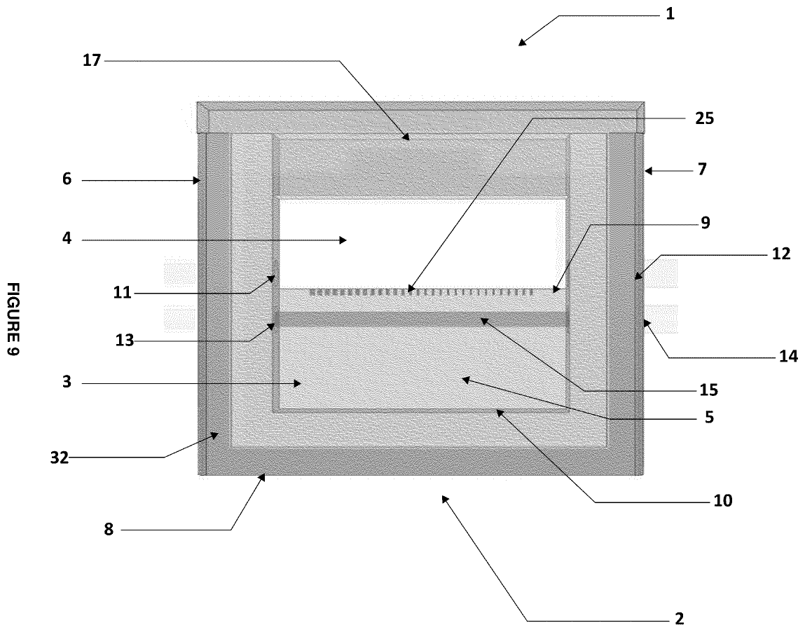

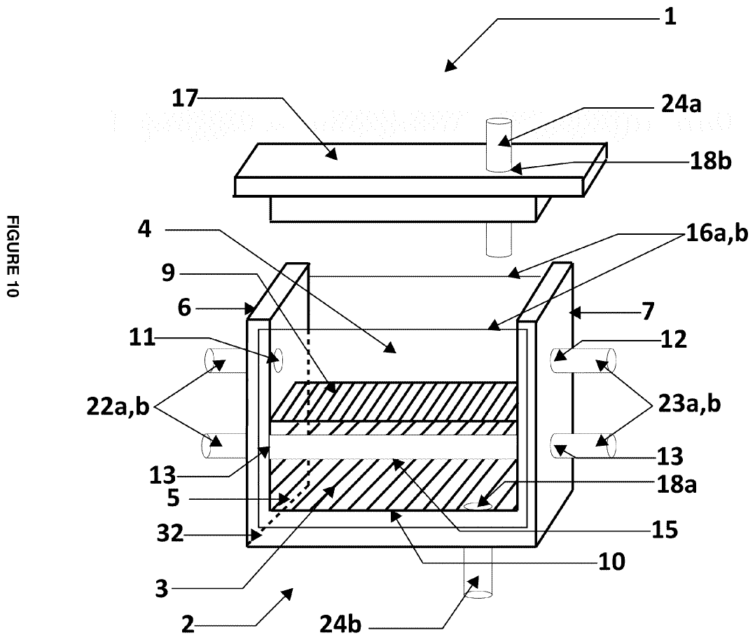

As shown on FIG. 8, the porous member (3) may fill partially the second zone (5). In such case a cavity (19) may be arranged between the second surface (10) and the frame (32). The cavity (19) is positioned in the bottom part of the chamber (2). The cavity (19) may be delimited by the bottom closure (8).

A porous member (3) filling completely the second zone (5) comprises a microchannel (15) in open connection with two ports of the second set of ports.

In one configuration, a porous member (3) may fill partially the second zone (5) and may comprise a microchannel (15) in open connection with two ports of the second set of ports, and the cavity (19) may be a closed cavity, not in open connection with two ports of the second set of ports arranged in the frame (32).

In a second configuration, a porous member (3) may fill partially the second zone (5) and may comprise a microchannel (15) in open connection with two ports of the second set of ports, and the cavity (19) may be in open connection with two additional ports of the second set of ports arranged in the frame (32).

In a third configuration, a porous member (3) may fill partially the second zone (5) and may not comprise a microchannel, and the cavity (19) may be in open connection with two ports of the second set of ports. arranged in the frame (32).

As shown on FIG. 8, the porous member (3) may comprise one or more, for example two or more, microchannel (15), each being in open connection with a pair of ports of the second set of ports arranged in the frame (32).

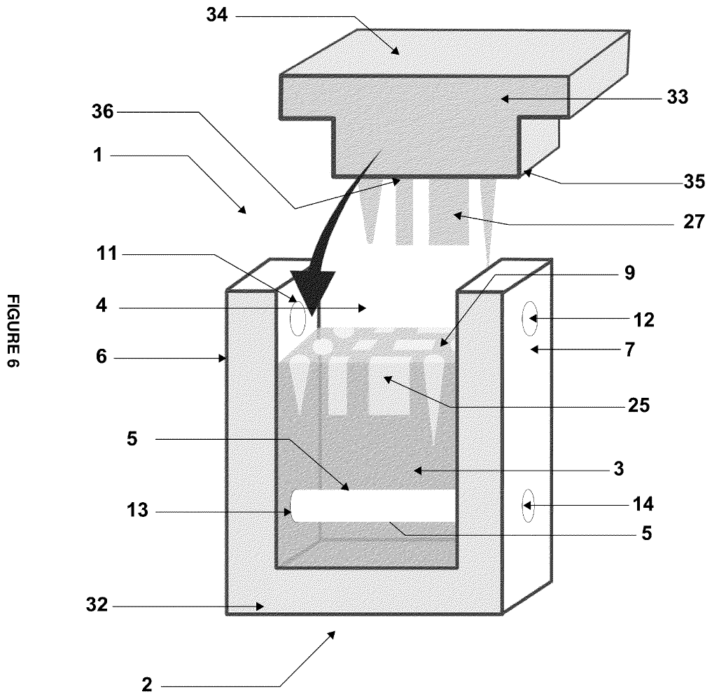

As shown on FIGS. 5 and 6, a microfluidic device (1) may comprise two opposite walls (16a,b—FIG. 5) and a frame (32) defining a chamber (2) comprising at least a first (4) and a second (5) zones:
the frame (32) may comprise a movable closure (33—FIG. 6) and at least a first (11, 12) and a second (13, 14) sets of ports, the first (11, 12) and second (13, 14) sets of ports comprising at least two ports (11, 12) or (13, 14) arranged in the frame (32) for fluid circulation within, respectively, the first (4) and the second (5) zone,
the movable closure (33) being arranged to extend at least partly within the chamber (2) through the first zone (4), the movable closure comprising an outward face (34) and an inward face (35), the inward face being arranged to extend at the interface of the first (4) and second (5) zones. The inward face (35) may comprise at least partly an imprinting face (36).

As shown on FIGS. 5 and 6, a microfluidic device (1) may comprise aframe (32) and two opposite walls (16a,b), the two opposite walls (16a,b) and the frame (32) delimiting together a chamber (2):
the chamber (2) comprising a first zone (4) and a second zone (5),
the frame (32) comprising a movable closure (33) and at least a first and a second sets of ports (11, 12, 13, 14),
the first set of ports comprising at least two ports arranged (11, 12) in the frame (32) for fluid circulation within the first zone (4) and the second set of ports comprising at least two ports (13, 14) arranged in the frame (32) for fluid circulation within the second zone (5), and
   the movable closure (33) comprising an outward face (34) and an inward face (35), the inward face (35) comprising an imprinting face (36), and the movable closure (33) extending within the chamber (2) through the first zone (4) up to an interface between the first zone (4) and the second zone (5).

The first zone (4) is filled with the movable closure (33). The interface between the first and second zones (4, 5) is delimited by the imprinting face (36) of the inward face (35).

The interface between the first zone (4) and the second zone (5) may be delimited by the first surface (9) of the porous member (3).

The movable closure (33) may be referred to as movable closure for imprinting. In some embodiments, a microfluidic device or a microphysiological system (MPS) as disclosed herein may comprise a closure devoid of the inward imprinting face and which can be referred to as a simple movable closure or a closure for sealing (17).

The second zone (5) of the chamber (2) may comprise:
a porous member extending in the chamber (2) and comprising a first surface (9) and a second surface (10) opposite to first surface, the first surface (9) delimiting the interface between the first zone (4) and the second zone (5) of the chamber (2), and the two ports (13, 14) of the second set of ports being open
(i) in a microchannel (15) extending through the porous member (3), or
(ii) in a cavity (19) arranged between the second surface (10) of the porous member (3) and the frame (32) of the chamber (2).

### Chamber

As shown for example on FIG. 5, chamber of a microfluidic device (1) or of a MPS as disclosed may comprise a frame (32) and two opposites walls (16a,b).

The frame may comprise at least two lateral walls (6, 7) arranged between the opposite walls (16a,b).

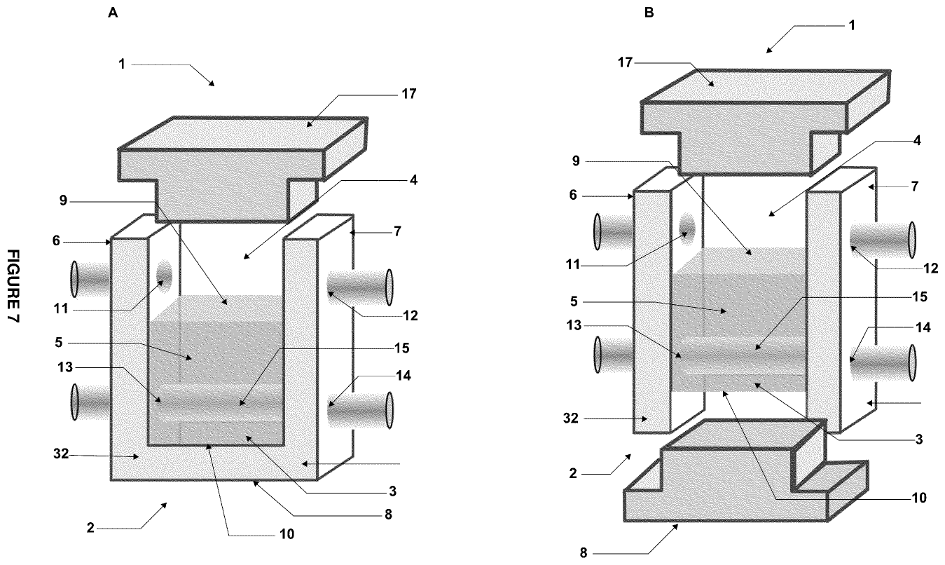

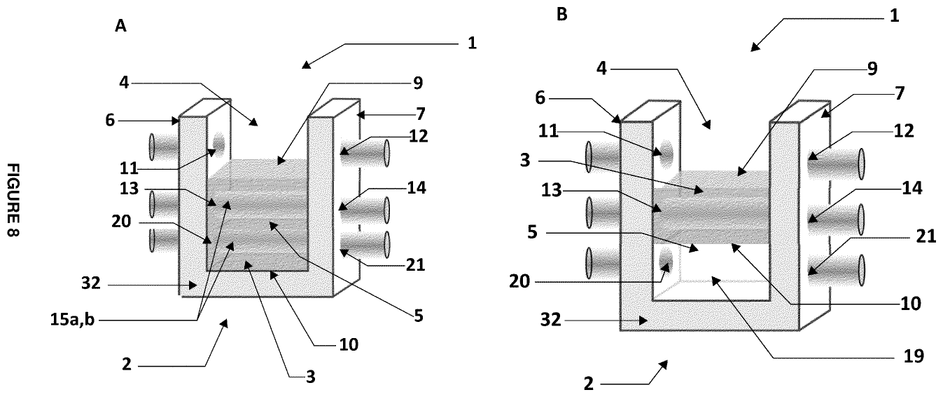

As shown for example on FIGS. 7A and 7B, the frame may comprise a top (17) and/or a bottom (8) closure. At least one of the top (17) and bottom (8) closure may be a movable closure.

As shown on FIG. 7A, the frame may comprise a closure (8) which can be a fixed closure integral with the lateral walls (6, 7). The lateral walls (6, 7) may be connected together by a top or a bottom closure, and for example are connected together by the bottom closure (8). In some embodiments, the lateral walls (6, 7) and the bottom closure (8) of the frame (32) may delimit a U-shaped frame.

The frame (32) of the chamber (2) may be made of any biocompatible material suitable for 3D printing or stereolithography. Examples of biocompatible materials include, but are not limited to, glass, silicon, silicones, polyurethanes, rubber, molded plastic, polymethylmethacrylate (PMMA), polycarbonate, polytetrafluoroethylene (TEFLON™), polyvinylchloride (PVC), polydimethylsiloxane (PDMS), polysulfone, photopolymerable materials based on polyurethane, polyacryltes, thiolenes, thermoplastic elastomers, and fluorinated thermoplastic elastomers.

In some embodiment, a frame (32) of the chamber (2) may be fabricated with PDMS (poly-dimethylsiloxane).

The frame (32) may comprise at least a first (11, 12) and a second (13, 14) sets of ports, the first (11, 12) and second (13, 14) sets of ports may each comprise at least two ports (11, 12) or (13, 14) arranged in the frame (32) for fluid circulation within, respectively, the first (4) and the second (5) zone.

The at least two ports (11, 12) of the first set of ports may be each opposite and symmetrically positioned in the frame (32) relatively to a vertical plan or may be each opposite and vertically and/or horizontally shifted one relatively to the other.

The at least two ports (13, 14) of the second set of ports may be each opposite and symmetrically positioned in the frame (32) relatively to a vertical plan or may be each opposite and vertically and/or horizontally shifted one relatively to the other.

As shown on FIG. 8A or FIG. 8B, in some embodiments, the microfluidic device (1) as disclosed herein may comprise (i) at least an additional microchannel (15a,b) in the first porous member (3) (FIG. 8A) and/or (ii) a cavity (19) (FIG. 8B) delimited between the second surface (10) of the first porous member (3) and the frame (32) of the chamber, and the frame (32) may optionally comprise at least an additional set of ports comprising at least two ports (20,21) arranged in the frame (32) for fluid circulation within said additional microchannel (15b) and/or said cavity (19) of the chamber (2).

A microfluidic device (1) discloses herein may comprise:
a second zone (5) comprising the porous member (3) comprising a microchannel (15a) and an additional microchannel (15b), or
a second zone (5) comprising (a) the porous member (3) comprising the microchannel (15) and (b) the cavity (19),
and the frame (32) comprises an additional set of ports comprising at least two ports (20, 21) arranged in the frame (32) for fluid circulation within the second zone (5) and being open in the additional microchannel (15a) or in the cavity (19)

In one configuration, a porous member (3) may fill completely the second zone (5) and may comprise one microchannel (15) in open connection with a pair of ports of the second set of ports. In such configuration there is no cavity (19).

In one configuration, a porous member (3) may fill completely the second zone (5) and may comprise two or more microchannel (15), each being in open connection with a pair of ports of the second set of ports. In such configuration there is no cavity (19).

In another configuration, a porous member (3) may fill partially the second zone (5) and may comprise a microchannel (15) in open connection with two ports of the second set of ports, and the cavity (19) may be a closed cavity, not in open connection with two ports of the second set of ports arranged in the frame (32).

In another configuration, a porous member (3) may fill partially the second zone (5) and may comprise a microchannel (15) in open connection with two ports of the second set of ports, and the cavity (19) may be in open connection with two additional ports of the second set of ports arranged in the frame (32).

In another configuration, a porous member (3) may fill partially the second zone (5) and may not comprise a microchannel, and the cavity (19) may be in open connection with two ports of the second set of ports arranged in the frame (32).

In another configuration, a porous member (3) may fill partially the second zone (5) and may comprise two or more microchannel (15), each being in open connection with a pair of ports of the second set of ports, and the cavity (19) may be in open connection with two additional ports of the second set of ports arranged in the frame (32).

In another configuration, a porous member (3) may fill partially the second zone (5) and may comprise two or more microchannel (15), each being in open connection with a pair of ports of the second set of ports, and the cavity (19) may be a closed cavity, not in open connection with two ports of the second set of ports arranged in the frame (32).

The ports of the second set of ports are arranged in the frame (32) to be in fluid connection with the microchannel(s) and/or the cavity (19).

In some embodiments, the chamber (2) may comprise a second porous member. The second porous member may comprise a first surface and a second surface opposed to the first surface. The first surface of the second porous member may be opposite and spaced from the second surface of the first porous member to define a volume in the chamber between the second surface of the first porous member and the first surface of the second porous member. The frame (32) may comprise optionally at least an additional set of ports comprising at least two ports arranged in the frame for fluid circulation within said volume of the chamber.

The at least two additional ports of the at least additional first set of ports may be each opposite and symmetrically positioned in the frame relatively to a vertical plan or may be each opposite and vertically and/or horizontally shifted one relatively to the other.

In some embodiments, the opposite walls (16a,b) may be, in whole or in part, transparent to visible light, UV light, infrared light, or X rays.

At least one or both of opposite walls (16a,b) may be coverslip of glass, quartz, or of polymer, such as PMMA, Polystyrene (PS), or Polycarbonate (PC).

The opposite walls (16a,b) may be of any suitable size, thickness, or shape, depending on the use of the microfluidic device. For example, the opposite walls (16a,b) may, independently of each other, have a square, a rectangular or a round shape. The size may vary from about 13×13 mm to about 16×16 mm for a square shape. The thickness may be of about 0.2 mm.

The opposite walls (16a,b) may be, independently of each other, plane or curved, such as convex or concave. In some embodiments the third and fourth walls are plane.

In some embodiments, the opposite walls (16a,b) may be, independently of each other, movably fixed onto the frame (32) of the chamber (2) or may be glued to the frame (32) of the chamber (2). In some embodiments, the opposite walls (16a,b) may be glued to the frame (32) of the chamber (2).

The surface of the inner side(s) of the opposite walls (16a,b) may be, independently of each other, chemically treated to enhance adhesion to the frame of the chamber, adhesion to the porous member, and/or adhesion of the culture cells. Examples of suitable chemical treatment may include treatment resulting in a hydrophilic or hydrophobic coating, treatment resulting in negatively or positively charged coating, coating with ITO (tin-doped indium oxide), silanization.

Surface treatment of the inner side(s) of the opposite walls (16a,b) may be used to impart specific properties to the walls, such as thermal or electric conductivity; porosity, for allowing gas entry or exit; deformability; micro-nanostructures; and combinations thereof.

In some embodiments, the opposite walls (16a,b) are silanized glass coverslips.

The opposite walls (16a,b) may comprise integrated sensor or device, such as optode for measuring gas such as O₂, CO₂; electrodes, for measuring electric current; optical sensor or device, such as LED or reflector; piezoelectric sensor, for measuring mechanical deformation; sensor for detecting volume and surface waves; biosensors, such as enzymes, antibodies, aptamers, DNA, or RNA; and combinations thereof.

In some embodiments, a microfluidic device (1) disclosed herein may comprise at least one light-conductive element, such as an optical fiber, able to bring light at predefined positions inside the chamber, or collect light therefrom, or a combination thereof.

In some embodiments, a microfluidic device (1) disclosed herein may comprise at least one optical assembly. The optical assembly may be used, for instance, to condition light to be applied in one zone of the chamber, or to condition and analyze light collected from a zone. The optical assembly can comprise, for instance, any or any combination of lenses, mirrors, filters, prisms, chromatographic elements, gratings, light-emitting devices, light sources, light-sensitive devices, diodes, photodiodes photomultipliers, cameras, light intensifiers, waveguides, microscope objectives, optical fibers, polarizers, and more generally any of optical components known in the art.

The opposite walls (16a,b) or light conducting elements, or optical components, allow observation by optical imaging or detection means, or photostimulation, inside of the zones of the chamber. A microfluidic device may be used for optical imaging, fluorescence, luminescence, photostimulation, light absorption, spectroscopy, chemiluminescence, electrochemiluminescence, or all kinds of optically activated chemical or biological reactions, such as light-induced transconformation, uncaging, polymerization, degradation, optogenetics, electro-optic surface modifications, and the like.

In some embodiments, the opposite walls (16a,b) may comprise electrically conducting elements associated in the invention's device. In some preferred embodiments, they are in electric connectivity with the interior of the chamber.

The conducting elements may be in direct electric contact with at least one zone of the chamber, or they may be insulated from the zones of the chamber in the microfluidic device.

In some embodiments, the conducting elements may comprise, or be part of, or be connected to, any electronic device, or any device involving the measure of a current, or the measure of a potential. As a non-exhaustive list of examples, the conducting elements may comprise pH sensors, ion-sensitive sensors, biosensors, chemical sensors, electrochemically active electrodes, piezoelectric elements, deformation sensors, position sensors, components of an impedancemetric sensor, temperature sensors, field effect transistors, and the like.

In some embodiments, the electrically conducting element may be a heating element.

In some embodiments, the electrically conducting element may be a solenoid, or a spire, generating a magnetic field.

In some embodiments, a microfluidic device (1) disclosed herein, for example the third and/or fourth walls, may comprise at least a magnetic element.

The magnetic element may be a wire of a magnetic material. The magnetic element may be a magnetic core of soft magnetic material, or a magnet.

### Porous Member

A microfluidic device (1) or a MPS as disclosed herein may comprise at least a first porous member (3). Optionally, a microfluidic device (1) or a MPS as disclosed herein may comprise at least a second porous member.

The first porous member (3), and optionally the at least second porous member, may be positioned between, and in contact with, the opposite walls (16a,b) and the lateral walls (6, 7) delimiting the chamber (2).

The porous member (3) comprises a first surface (9) and a second surface (10). The second surface (10) is opposite to the first surface (9). The second surface (10) is positioned over the bottom closure (8) of the frame (32).

In some embodiments, the first zone (4) is delimited by the first surface (9) of the first porous member (3), the part of the frame (32) comprising the at least first set of at least two ports (11, 12), and optionally, an inward face of a top closure (17).

The first zone (4) may be positioned between a part of the lateral walls (6, 7), the part of the first lateral wall (6) comprises a first port (11) and the part of the second lateral wall (7) comprises a second port (12). The first zone (4) comprises a base side. The base side is the first surface (9) of the porous member (3).

In some embodiments, the second zone (5) is delimited by the second surface (10) of the first porous member (3), the part of the frame (32) comprising the at least second set of at least two ports (13, 14), and optionally an inward face of a bottom closure (8).

The second zone (5) may be positioned between a part of the lateral walls (6, 7), the part of the first wall (6) comprises a third port (13) and the part of the second wall (7) comprises a fourth port (14).

A porous member (3) may be any element configured for allowing passage and diffusion of various materials, such as fluids, gas, biological materials or cells from the first zone to the second zone and reciprocally

The porous member (3) may be a monolayer or a multilayer member. For example, a multilayered porous member may comprise several, at least two, distinct layers of different type of materials, such as polymers, or of the same material, such as polymer at different concentrations.

In some embodiments, the first surface (9) of the first porous member (3) may be a cell culturing surface.

In some embodiments, the cell culturing surface may comprise reliefs (25).

The reliefs (25) may form a regular pattern. The reliefs may be microcavities or protruding micro-reliefs. The reliefs may be regularly spaced to each other on the cell culturing surface.

In some embodiments, a pattern may be within the porous member (3).

In some embodiments, with a multilayer porous member, a pattern may be in a volume within the porous member (3), at an interface of two layers.

In some embodiments, with a multilayer porous member, a pattern may be present across at least one, two, or more layers.

A porous member (3) may be homogenous or heterogeneous.

A porous member (3) may be a monolayer or a multilayer porous member.

A homogenous porous member may be a porous member made with a single type of polymer with a homogenous concentration across the volume of the matrix. In such case, the porous member is a monolayer.

A heterogeneous porous member may be a porous member made with a single type of polymer but with a gradient of concentration of polymer, increasing and/or decreasing from the bottom face towards the upper face of the porous member and/or from the first wall towards the second wall of the chamber.

Advantageously, a porous member may be transparent to at least one wavelength in the range from infrared to UV wavelengths.

A porous member may be a 4D porous member or 4D matrix. A 4D matrix is a matrix whose shape, property, and functionality are able to self-transform when exposed to a predetermined stimulus. 4D printed porous member, such as hydrogels, are known in the art (Dong et al., Advanced Materials Technologies, 2020).

A porous member (3) may be used as support for culturing cells. The use of porous member, such as hydrogel matrices as extracellular matrix analogs, is well-known in the art (González-Díaz et al., Gels. 2016). The cells may be cultured either on the surface of the porous member or within the volume of the porous member.

In some embodiments, the first porous member (3), and optionally the at least second porous member, are a porous matrix. A porous matrix may be for example a hydrogel matrix or a ceramic.

In some embodiments, a porous member (3) may be a hydrogel matrix.

In some embodiments, a porous member (3) may be a hydrogel matrix made of a permanent or chemical gel, such as PEG diacrylate, polyacrylamide, gelatin-methacrylate, or hyaluronic acid-methacrylate, or methacrylate.

In some embodiments, a porous member (3) may be a hydrogel matrix made of a reversible or physical gel, such as collagen, gelatin, or hyaluronic acid.

The chemical composition, density, and hydrophobicity of the polymers can make the hydrogel vary in consistency from viscous fluids to rigid solids. The porosity of the hydrogel can be controlled easily by tuning the density of cross-links.

Hydrogel matrices may be homopolymer hydrogels, i.e., made of one type of hydrophilic monomer unit; copolymer hydrogels, i.e., consisting of two comonomer units, one of which must be hydrophilic; or multipolymer hydrogels, i.e., produced from three or more comonomers reacting together.

Also, hydrogels may be neutral, anionic, cationic, or ampholytic hydrogels. Also, hydrogels may be amorphous hydrogels, i.e., non-crystalline containing randomly arranged macromolecular chains; semicrystalline hydrogels, i.e., include a mixture of amorphous and crystalline phases possessing dense regions of ordered macromolecular chains; or hydrogen-bonded structures, i.e., the hydrogel network is based on electrostatic interactions.

In some embodiments, a hydrogel matrix may be obtained at least one polymer suitable to be cross-linked in hydrogel matrix, when placed in suitable conditions. Cross-linking may be obtained chemically or by photopolymerization orthermo-polymerization though the transparent portion of the opposite walls (16a,b).

Hydrogel matrices can be made from at least one type of polymer.

In some embodiments, a hydrogel matrix may be made of at least two, at least 3, at least four, at least five, or more, types of polymers. In some embodiments, a hydrogel matrix may be made of at least two types of polymers.

A hydrogel matrix may be homogenous or heterogeneous.

A hydrogel matrix may be a monolayer or a multilayer hydrogel matrix.

For example, a homogenous hydrogel matrix may be a hydrogel matrix made with a single type of material, e.g., a polymer, with a homogenous concentration across the volume of the matrix. In such case, the hydrogel matrix is a monolayer.

For example, a heterogeneous hydrogel matrix may be a hydrogel matrix made with a single type of material, e.g., a polymer but with a gradient of concentration of the material, increasing and/or decreasing from the bottom face towards the upper face of the hydrogel matrix and/or from the first wall towards the second wall of the chamber.

A heterogeneous hydrogel matrix may be a composite hydrogel matrix, i.e., a hydrogel matrix made with at least two types of materials, e.g., polymers, each material with a single concentration, or at least one with a gradient of concentration, increasing and/or decreasing, from the second surface towards the first surface of the hydrogel matrix and/or from one lateral wall towards the opposed lateral wall of the frame.

A heterogenous hydrogel matrix may be a multilayer matrix.

A heterogenous or composite hydrogel matrix may be composed of at least two layers of materials, e.g., polymers, a first layer being made with at least a first type of material, e.g., polymer, and a second layer being made with at least a second type of material, e.g., polymer. For example, a composite hydrogel matrix may comprise a first layer of a first type of material, e.g., polymer, a second layer of a second type of material, e.g., polymer, and optionally a third layer, or more, of a third, or more, type of material, e.g., polymer. A material, e.g., polymer may have a constant concentration or a gradient of concentration throughout its associated layer.

The layers of materials, e.g., polymers, may be arranged horizontally or vertically.

The layers of materials, e.g., polymers, may be arranged as stacks of polymers with a constant concentration of material, e.g., polymer, within a stack or may be arranged with a gradient of concentration of material, e.g., polymer, with a stack, increasing and/or decreasing, from the second surface towards the first surface of the hydrogel matrix, or from one lateral wall towards the opposed lateral wall of the frame.

In some embodiments, a hydrogel matrix is a composite matrix comprising at least two layers of a first and of a second type of materials, e.g., polymers. The layers may be arranged horizontally.

In some embodiment, the concentration of the material, e.g., polymer, is constant (or homogeneous) in the layer.

A hydrogel matrix may be a synthetic, a natural, or a physical gel, or a thermosensitive or a photosensitive material, an alginate, a chemosensitive gel.

A material or element suitable to form a hydrogel matrix may be, for example, carboxymethyl cellulose, possibly cross-linked, a modified starch, alginate, pectin, hyaluronic acid, collagen, matrigel, decellularized matrix, gelatin, polyacrylamide, polyethylene glycol, hydrogel of poly (2-hydroxyethyl methacrylate) (pHEMA), GELMA, PDMS, Nylon, Matrigel, Cultrex, and combination thereof.

Hydrogel matrices made from synthetic hydrophilic polymers, such as polyacrylamide or polyethylene glycol, may not support cell adhesion. To make hydrogels cell-adhesive one may incorporate a cell adhesive material, such as peptides or proteins, into the hydrogel network. The biofunctionalization of hydrogels may be achieved through bioconjugation, such as coupling between —NHS and amine groups, Michael-type addition, thiol-acrylate reaction, copolymerization, or coating.

In some embodiments, the hydrogel matrix may be a polyacrylamide hydrogel coated with a cell-adhesive material. A cell adhesive material may be a hydrogel matrix.

A cell-adhesive material may be RGD peptides; vitronetin-derived peptide; proteoglycan moieties, such as chondroitin sulfate (CS) moieties, hyaluronic acid moieties, or heparin; collagen; and combinations thereof.

A suitable collagen may be a collagen of type I, II, III, IV, V, VI, VII, VIII, IX, X, XI or XII.

Acrylamide may be advantageously used to prepare a hydrogel matrix since it is chemically inert, electrically neutral, hydrophilic, and transparent for optical detection at wavelengths greater than 250 nm. Polyacrylamide gels may be prepared by free radical polymerization of acrylamide and a comonomer crosslinker such as bis-acrylamide.

Polymerization may be initiated by ammonium persulfate (APS) with tetramethylethylenediamine (TEMED) as the catalyst. Riboflavin (or riboflavin-5′-phosphate) may also be used as a source of free radicals, often in combination with TEMED and APS. Polymerization speed depends on various factors, such as monomer and catalyst concentration, temperature and purity of reagents.

In some embodiments, a hydrogel matrix is a composite matrix comprising at first layer of a polyacrylamide hydrogel and of a second layer of collagen I. The layers may be arranged horizontally. The first layer may be positioned below the second layer. In some embodiment, the upper face of the hydrogel matrix is composed of a layer of collagen.

A hydrogel of polyacrylamide may contain from about 1% to about 15% of acrylamide, from about 3% to about 12%, from about 4% to about 10%, from about 6% to about 8%, and for example from about 4% to about 10%, or may contain 4% or 10% of acrylamide.

A hydrogel of polyacrylamide may contain about 2%, 3, 4, 5, 6, 7, 8, 9, 10, 11, 12, 13, 14, or about 15% of acrylamide.

A hydrogel of polyacrylamide may contain from about 8 to about 12% of acrylamide.

A hydrogel of polyacrylamide may contain from about 0.01% to about 1% of bis-acrylamide, from about 0.05% to about 0.75%, from about 0.1% to about 0.50%, from about 0.15% to about 0.40%, from about 0.2% to about 0.3% of bis-acrylamide.

A hydrogel of polyacrylamide may contain about 0.01%, 0.05, 0.1, 0.2, 0.3, 0.4, 0.5, 0.6, 0.7, 0.8, 0.9 or about 1% of bis-acrylamide.

After preparing and casting a polyacrylamide hydrogel in a microfluidic device as disclosed herein, the first surface of the hydrogel may be coated with a cell adhesive coating material.

A gel of collagen may contain from about 0.1% (0.1 mg/mL) to about 15% (15 mg/mL) of collagen, from about 0.5% to about 10%, from about 0.8% to about 12%, from about 1% to about 10%, from about 1.5% to about 8%, from about 2% to about 6%, or from about 3% to about 5%, may contain 1% or 5% of collagen.

A gel of collagen may contain about 0.1%, 0.5, 1, 1.5, 2, 2.5, 3, 4, 5, 6, 7, 8, 9, 10, 11, 12, 13, 14 or about 15% of collagen.

A gel of collagen may contain from about 1 to about 5% of collagen.

A porous member (3) may comprise a hydrogel of polyacrylamide coated with collagen, where the amount ratio of polyacrylamide to collagen is from about 1.2:1 to about 1000:1, from about 2:1 to about 800:1, from about 4:1 to about 600:1, from about 8:1 to about 300:1, from about 10:1 to about 200:1, from about 15:1 to about 150:1, from about 20:1 to about 100:1, or is from about 20:1 to about 40:1.

A porous member (3) may comprise a hydrogel of polyacrylamide coated with collagen, where the amount ratio of polyacrylamide to collagen is at about 20:1.

A suitable porous member (3) may have a Young's modulus ranging from about 0.1 kPa to about 150 kPa, from about 0.5 kPa to about 125 kPa, from about 1 kPa to about 100 kPa, from about 1.5 kPa to about 80 kPa, from about 2 kPa to about 60 kPa, from about 2.5 kPa to about 40 kPa, from about 3.0 kPa to about 25.0 kPa, from about 3.5 to about 22.0 kPa, from about 4.0 to about 20.0 kPa, from about 5.0 to about 18.0 kPa, from about 6.0 to about 15.0 pKa, from about 8.0 to about 12.0 kPa.

A suitable porous member (3) may have a Young's modulus of about 0.1, 0.5, 1, 1, 5, 2, 2.5, 3, 3.5, 4, 5, 6, 7, 8, 9, 10, 11, 12, 13, 14, 16, 17, 18, 19, 20, 21, 22, 23, 24, 25, 30, 40, 50, 60, 70, 80, 90, 100, 110, 120, 130, 140, or about 150 kPa.

A suitable porous member (3) may have a Young's modulus of about 3.5 to about 17 kPa.

A suitable porous member (3) may have a Young's modulus of about 15 to about 17 kPa.

The Young's modulus of porous member may be measured by atomic force microscopy (AFM).

An AFM measurement may be performed using Borosilicate Glass Particle (10 μm) on silicon nitride cantilevers (PT.BORO.SN.10, Novascan Technologies) with a nominal spring constant of 0.06 N/m mounted on a NanoWizard Ill AFM (JPK Instruments) coupled to an inverted optical microscope (Axiovert 200, Carl Zeiss).

To ensure reproducibility in force application and measurement, the cantilever sensitivity and spring constant should be calibrated before each measure using the JPK Instrument software using the thermal noise method. Force-distance curves may be recorded in contact mode in liquid at 2 Hz (1 second per approach-retract force curve) with an applied force of 1 nN.

Each hydrogel was tested at 4 locations with about 250 force curves acquired per location and 512 data points per curve. The measured area was 150 μm×150 μm per location.

In some embodiments, the porous member (3) may comprise at least one probe or a sensor. A probe may be suitable to obtain measure on mechanical constraints, pressure, shear stress, deformation resulting from the culture cells.

A probe may be a colored or fluorescent dye, a metallic particle, or a magnetic particle, such as fluorescent micro or nanoparticle, quantum dot, or RX absorbing probe such as KI. Such probe may emit a signal representative of the change occurring in the porous member or on the surface of the porous member as a result of the cell culture or the interactions of the cells with the surrounding environment. For example, such probes may be used to detect and measure gel stress exerted by cells.

As above indicated, and as shown on FIG. 5, 6 or 9, in some embodiments, reliefs (25) may be present on the first surface (9) of the porous member (3).

In some embodiments, the reliefs (25) may form a regular pattern. The reliefs (25) may be microcavities or protruding micro-reliefs. The reliefs (25) may be regularly spaced to each other on the first surface.

In some embodiments, the first surface (9) of the first porous member (3) may delimit a cell culturing surface.

In some other embodiments, reliefs (25) may be present in a volume of the porous member (3). In such embodiments, the reliefs (25) are inside the volume delimited by the porous member (3).

In some embodiments, with a multilayer porous member (3), reliefs (25) may be present in a volume within the porous member (3), at an interface of two layers.

The provision of reliefs (25) on the first surface (9) of or in a volume in the porous member (3) may be carried out by molding, sculpting, imprinting and/or embossing the matrix with different reliefs, patterns or 3D-structures at the first surface (9) of the porous member (3) or within the volume of the porous member (3).

In some embodiments, reliefs (25) such as a regular pattern may be imprinted or embossed on the first surface (9) of a porous member (3).

In some embodiments, the provision of reliefs (25) may involve the presence on the first surface (9) or in a volume of the porous member (3) of, for instance, protrusions, recesses, ridges, posts, wells, and the like.

In some other embodiments, the provision of reliefs (25) may involve imparting the first surface (9) or a volume of the porous member (3) with different properties, such as optical properties, wetting properties, chemical properties, adhesion properties, electric properties, or magnetic properties.

In some embodiments, the reliefs (25) may comprise regularly spaced reliefs or a regular pattern. The regularly spaced relief may be cylindrical reliefs. The cylindrical reliefs may be comprised of cylindrical having a height of about 400 μm, a diameter of about 50-100 μm, and being spaced from each other of about 400 μm.

In some embodiments, reliefs (25), such as a regular pattern, may be imparted by imprinting or embossing with a mold or a movable closure (33) for imprinting, as disclosed herein.

The provision of reliefs (25) on the first surface (9) of a porous member (3) may be made by contacting the first surface (9) of a porous member (3) with a mold or a movable closure (33) as disclosed herein bearing at its surface, the inverted representation of the reliefs (25) to be imprinted or embossed. In such case, the reliefs (25) are imprinted or embossed. The imprinting may be made by application of the mold or movable closure (33) onto the first surface (9) of the porous member (3) during the curing or hardening of the material forming the porous member (3).

For example, a microfluidic device (1) containing a porous member (3) bearing at its first surface (9) some reliefs (25) may be manufactured with a method comprising the steps of: casting in the chamber (2) at least one material suitable to form a porous member (3), applying a mold or movable closure (33) bearing an inverted representation of the reliefs (25) to be imprinted onto the first surface (9) of the casted material suitable for forming a porous member (3), hardening or curing the material in conditions suitable for obtaining a porous member (3), removing the mold or movable closure (33), and obtaining a porous member (3) with reliefs (25) imprinted on the first surface (9).

In some embodiments, a porous member (3) may comprise at least two layers of different materials. In some embodiments, a porous member (3) may be prepared by casting, in the chamber (2) of a microfluidic device (1) disclosed herein, at least a first material, such as a first polymer, suitable to form a first layer of a porous member (3), and at least a second material, such as a second polymer, suitable to form a second layer of the porous member (3), the second material being casted over the first material, applying a mold or movable closure (33) bearing an inverted representation of the reliefs (25) to be imprinted on the upper face of the second casted material, hardening or curing the first and second materials in conditions suitable for obtaining a porous member (3), removing the mold or the movable closure (33) for imprinting, and obtaining a porous member (3) with reliefs (25) imprinted on the first face (9). In some embodiments, the thickness of the second layer of material may be such that the application of the mold or movable closure (33) for imprinting or embossing imparts the reliefs across the second and first layers of the porous member (3).

In some embodiments, a porous member (3) with at least a first and second layers of materials suitable to form a porous member, such as first and second layers of polymers, may be imparted with reliefs (25) at an interface of the two layers.

In some embodiments, with a multilayer porous member (3), reliefs (25) may be present across at least one, two, or more layers.

In some embodiments, a porous member (3) with at least a first and second layers of materials suitable to form a porous member, such as a first a second layers of polymers, may be imparted with reliefs (25) across the two layers.

In some embodiments, a porous member (3) may be prepared by casting in the chamber (2) a first layer of a first type of a material suitable to form a porous member, such as a first polymer, for example a solution of acrylamide/bis-acrylamide, casting in the chamber (2) a second layer of a material suitable to form a porous member, such as a second type of polymer, for example a solution collagen, the second layer being positioned over the first layer, applying a mold or movable closure (33) for imprinting, the mold or the movable closure (33) bearing an inverted representation of the reliefs (25) to be imprinted for imparting reliefs (25) across the first and second layers, hardening or curing the first and second materials in conditions suitable for obtaining a porous member (3), removing the mold or the movable closure (33), and obtaining a porous member (3) with reliefs (25) imprinted on the first surface (9).

Alternatively, a porous member (3) with at least two layers of materials may be prepared by casting a first layer of a first type of a material suitable to form a porous member, such as a first polymer, for example a solution of acrylamide/bis-acrylamide, applying a mold or movable closure (33) for imprinting, the mold or the movable closure (33) bearing an inverted representation of the reliefs (25) to be imprinted, the inverted representation of the reliefs being coated with a layer of a material suitable to form a second layer of the porous member, such as a second type of polymer, for example collagen, hardening or curing the first material, and optionally the second material, in conditions suitable for obtaining a porous member (3), removing the mold or the movable closure (33), and obtaining a porous member (3) with two layers of materials and bearing reliefs (25) imprinted on the first surface (9).

In some embodiments, reliefs (25) in the volume of the porous member (3) with at least two layers of materials may be obtained with a method comprising the steps of: casting at least a first material suitable to form a first layer of a porous member (3), such as a first polymer, applying a mold or a movable closure (33) bearing an inverted representation of the reliefs to be imprinted onto the first surface of said first material, hardening or curing said at least first material in conditions suitable for obtaining a first layer of the porous member (3), removing the mold or movable closure, casting at least a second material suitable to form a second layer of a porous member (3), such as a second polymer, hardening or curing said at least second material in conditions suitable for obtaining a second layer of the porous member (3), and obtaining a porous member (3) with reliefs in a volume of the porous member.

In some embodiments, the first and second materials may be of same or different type. For example, they may of different types, such as (i) acrylamide/bis-acrylamide to obtain a polyacrylamide hydrogel, and (ii) collagen.

Alternatively, a pattern may be obtained at the upper face of or within a volume in the porous member by microdissection with a laser beam, such as disclosed in Nikolaev et al., (Nature. 2020; 585(7826):574-578).

A pattern may be obtained at the upper face of or within the porous member by 3D printing of the porous member, for example as disclosed in Wang et al., (Biomaterials. 2017; 128:44-55).

As above indicated, and illustrated on FIG. 5, 6 or FIG. 7, a porous member (3) may comprise a microchannel (15) having a first end connected with a first port (13) in a first wall (6) of the frame (32) and a second end open in a second port in a second wall (7) of the frame (32).

As shown in FIG. 8A, in some embodiments, a porous member (3) may comprise, further to a first microchannel (15a) at least a second microchannel (15b). Each of the microchannels have a first and a second ends. The first ends of the microchannels are each connected to a port (13a,b) in a first wall (6) of the frame (32) of the chamber (2).

The second ends of the microchannels are each connected to another port (14a,b) in the second wall (7) of the frame (32).

In some, a porous member (3) may comprise a network of microchannels. A network of microchannels may be obtained either by positioning an elongated member with multiple branches or multiple elongated members.

A microchannel (15) may be linear or non-linear. A “non-linear” microchannel refers to a flow path or microchannel having a longitudinal axis that deviates from a straight line along its length by more than an amount equal to the minimum cross-sectional dimension of the microchannel (15) or flow path. A “longitudinal axis” of a microchannel (15) refers to an axis disposed along the entire length of such a microchannel (15), which is coextensive with and defined by the geometric centerline of the direction of any bulk fluid which would flow through the microchannel (15) should such microchannel be arranged for a fluid to flow therethrough.

A microchannel (15) may have a multiplicity of shapes, topologies and sizes. As non-limiting examples, a microchannel can have circular, square, parallelepipedal (cuboid), ribbon-like, or have more complex shapes. In some embodiments, a microchannel (15) has a circular section on at least part of its length. In some embodiments, a microchannel (15) may have a constant cross-section. In some other embodiments, a microchannel (15) may have a non-uniform or non-constant cross-section.

In some embodiments, a microchannel (15) may comprise between its first and second ends at least one bifurcation and at least one anastomosis. In some embodiments, a microchannel (15) may comprise between its first and second ends a plurality of bifurcation and anastomosis. In some embodiments, a microchannel (15) may comprise between its first and second ends a plurality of interconnected microchannels. Such embodiments allow obtaining between the first and second ends of a microchannel a blood-vessels like network.

In some embodiments, a blood-vessels like network may comprise a single end in a first wall (6) of the frame (32) and at least two or more ends in a second wall (7) of the frame (32), or reciprocally.

A microchannel (15) may comprise at least a part in which it is elongated in at least one direction, said direction defining a main axis, in order to transport fluid along their main axis.

In some embodiments, a microchannel (15) may also be chamber-like or comprise a chamber-like zone.

In some embodiments, at least a part of the inner face a microchannel (15) may be coated with a material such as a metal or a polymer. The coating of part or all the inner face allows modulating or conferring differentiated properties of the microchannel (15) with regard to porosity, for example with regard to gas, drugs, or biological molecules diffusion, or with regard to mechanical or electrical properties.

In some embodiments, an inner face of a microhannel (15) may be porous to fluids, such as gas or liquids, or to cells.

Examples of material suitable for coating the inner face of a microchannel (15) may be metal, cellulose, PTFE, or textile fibers.

In some embodiments, a device as disclosed herein may comprise one microchannel (15) in the porous member (3) as a second compartment. The microchannel (15) may have a constant cross-section. The microchannel may have a first end in the first wall (6) and a second end in the second wall (7) of the frame (32) of the chamber (2).

### Closures

As shown on FIG. 6, 7A, 7B or on 9, in some embodiments, a microfluidic device (1) as disclosed herein may comprise at least one movable closure (8, 17, or 33). A closure may be a closure (8, 17) for sealing the chamber or a closure (33) for (sealing and) imprinting.

The closure (8, 17 or 33) comprises an inward face (35) and an outward face (34). The inward face (35) is facing the inside of the chamber (2). The outward face (34) is facing the exterior of the chamber (2).

A movable closure (33) for imprinting may be arranged to extend at least partly within the chamber (2) through the first zone (4), the movable closure (33) for imprinting may comprise an outward face (34) and an inward face (35), the inward face being arranged to extend at the interface of the first (4) and second (5) zones.

A movable closure (33), suitable for a microfluidic device (1) as disclosed herein, may comprise an outward face (34) and an inward face (35), the inward face (35) comprising an imprinting face (36), and the movable closure (33) being arranged for be able to extend within the chamber (2) through a first zone (4) up to an interface with a second zone (5).

In some embodiments, a top closure (17) and/or a bottom closure (8) may comprise an inward face (35) arranged to be an imprinting face (36). In some embodiments, an inward face (35) arranged to be an imprinting face (36) is the inwards face of a top closure (33).

The inward face may (35) be arranged to be contacted with the surface, first or second (9, 10), of a porous member (3).

In some embodiments, the inward face (35) may comprise at least partly an imprinting face (36). The inward face may be arranged to be an imprinting face (36). The inward face (35) may be arranged to be contacted with the first surface (9) of a porous member (3).

In some embodiments, the inward face (35) of a movable closure (33) for imprinting is in closed contact with the first surface (9) of the first porous member (3). In some other embodiments, the inward face (35) of a movable closure (33) for imprinting is in closed contact with the second surface (10) of the first porous member (3). The movable closure (33) for imprinting may be a top and/or a bottom closure (8, 17).

The imprinting face (36) may comprise a plurality of reliefs protruding structure(s) (27) intended to imprint or emboss reliefs (25) on the first surface (9) or the second surface (10) of a porous member (3). The protruding structures (27) delimit together inverted reliefs of the reliefs (25) intended to be imprinted or embossed on a surface (9, 10) of the porous member (3).

The reliefs or protruding structures (27) may be of any shapes and of any dimensions. The reliefs or protruding structures (27) may comprise identical, repeated, protruding structures or may comprises protruding structures differing from each other in shape and/or in dimensions. The protruding structures may be identical and repeated in part and may be different from each other, in shape and/or in dimensions, in part.

The imprinted face (36) may comprise repeated, regularly spaced, structures or reliefs or a regular pattern.

In some embodiments, the protruding structures (27) may be an inverted crypt-like pattern. The protruding structures (27) defining an inverted crypt-like pattern may identical, repeated, structures.

The regularly spaced relief or structure may be cylindrical reliefs. The cylindrical reliefs may be comprised of cylindrical having a height from about 350 μm to about 450 μm, or of about 400 μm, a diameter of about 50-120 μm, or from about 90 μm to about 110 μm, for example of about 100 μm, and being regularly spaced from each other from about 350 μm to about 450 μm, or of about 400 μm. The imprinted face (36) may comprise protruding structures (27) delimiting an inverted pattern intended to be imprinted or embossed on the first or second surface (9, 10) of the porous member (3), depending on whether the closure is a top (17) or a bottom closure (8). In some embodiments, the pattern is on the inward face (35) of a top closure (17) and is intended to be imprinted or embossed on the first surface (9) of the porous member (3).

A movable closure (8, 17) may be arranged to extend at least partly within the chamber (2) through the first zone (4) or the second zone (5). A closure (8, 17) is arranged to seal the chamber (2) to prevent any leakage of fluids during the use of the microfluidic device (1). The inward face of a closure (8, 17) does not extend in the chamber to the point facing and obstructing the ports arranged in the frame (32) of the chamber (32).

When in place, a movable closure (8, 17) may be arranged to hermetically seal the chamber. When used with a closure, the expression “hermetic sealing” intends to refer to a closure that prevent unintentional exit (or leakage) of materials, such as fluid or gas, from the chamber or that prevent unintentional entry of material, e.g., entering of contaminants, in the chamber.

A closure (8, 17) may be made of any biocompatible material suitable for 3D printing or stereolithography. Examples of biocompatible materials include, but are not limited to, glass, silicon, silicones, polyurethanes, rubber, molded plastic, polymethylmethacrylate (PMMA), polycarbonate, polytetrafluoroethylene (TEFLON™) polyvinylchloride (PVC), polydimethylsiloxane (PDMS), polysulfone, glass, silicon, silicones, polyurethanes, rubber, molded plastic, polymethylmethacrylate (PMMA), polycarbonate, polytetrafluoroethylene (TEFLON™), polyvinylchloride (PVC), polydimethylsiloxane (PDMS), polysulfone, photopolymerable materials based on polyurethane, polyacryltes, thiolenes, thermoplastic elastomers, or fluorinated thermoplastic elastomers.

In some embodiments, a closure, for example a closure (8, 17), may be porous to gas. Closures made with a material porous to gas allow exchanging gas such as oxygen of carbon dioxide during cell cultures.

In some embodiments, a closure, for example a closure (8, 17), may be made with a hydrogel-type material allowing the diffusion, inside or outside the microfluidic device or the microphysiological system, of compounds or biological molecules.

In some embodiments, a closure, for example a closure (8, 17), may comprise a conducive material. A closure may be used as electrodes. Such embodiment may be used for trans epithelial electrical resistances (TEER) measurements.

As shown on FIG. 10, in some embodiments, a base closure (8) and/or a top closure (17) may comprise at least one port (18a,b).

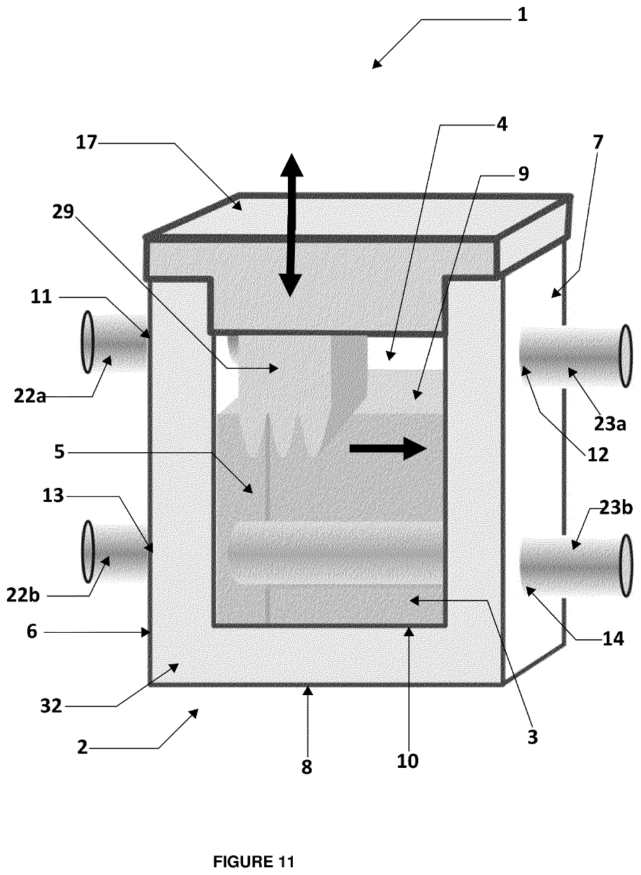

As shown on FIGS. 11, 12 and 13, in some embodiments, a top closure (17) may comprise a protruding element (29, 30, 31) intended to be positioned in contact with the porous member (3) and to exert a mechanical constraint or pressure on the porous member (3), for example on the first surface (9) of the porous member (3). The protruding element (29, 30, 31) may be of any shape and any dimensions. The protruding element (29, 30, 31) may be fixed or movable (or actionable).

The mechanical constraint may be uniformly exerted on all the area first surface (9) of the porous member (3) or may be exerted only on a part or on a plurality of parts of the area of the first surface (9) of the porous member (3). The dimensions and shape of the protruding element (29, 30, 31) may be such that the mechanical constraint or the mechanical pressure is exerted on the surface of the porous member (3) or in the volume of the porous member (3).

The protruding element (29, 30, 31) may be fixed or may be movable either vertically, perpendicularly to the plane of the first surface (9) of the porous member (3) or horizontally, parallel to the plane of the first surface (9) of the porous member (3).

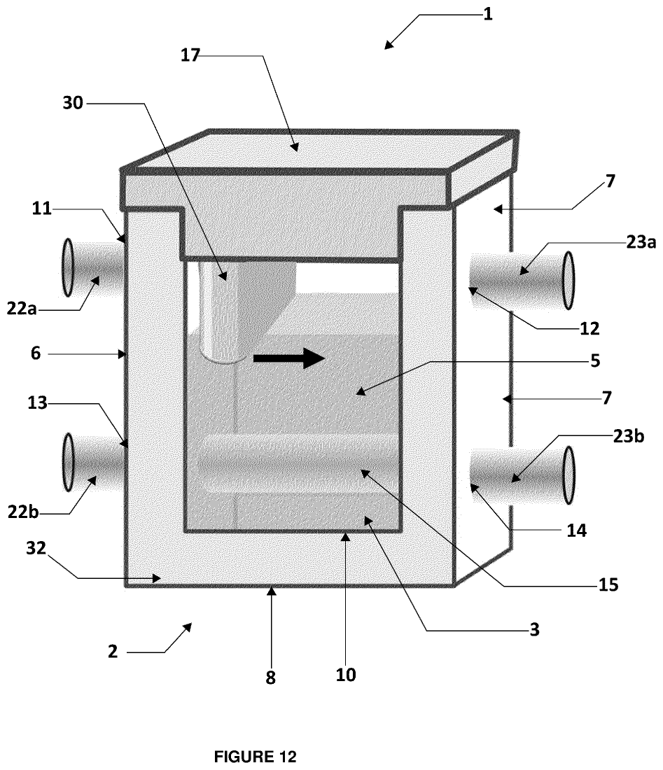

As shown on FIG. 12, in some embodiments, a protruding element (29, 30, 31) may be a flat, elongated, protruding element, such as a blade (31). The blade (31) may be movable in a direction parallel to the plane of the first surface (9) of the porous member (3) (horizontally).

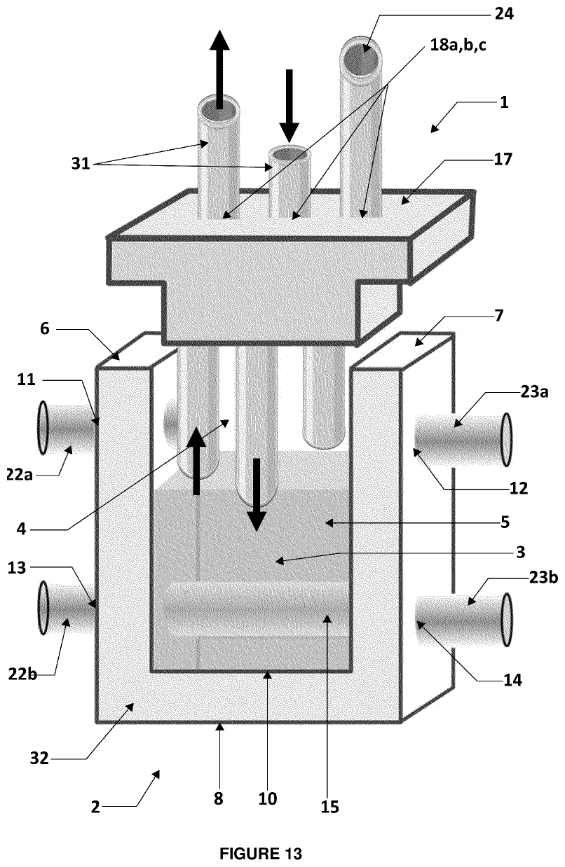

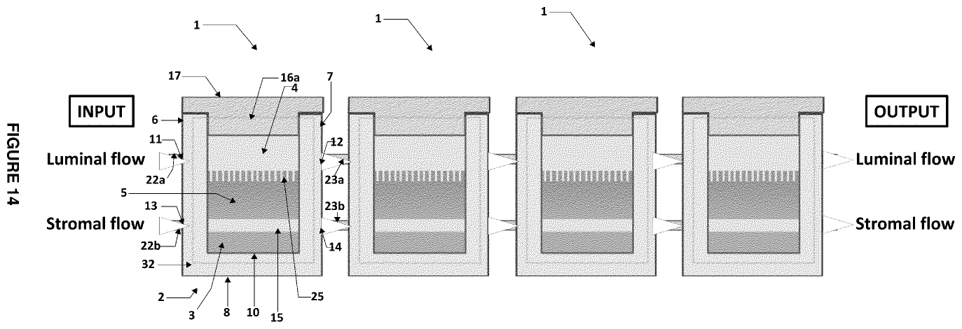

As shown on FIG. 13, in some embodiments, the protruding element may be plunger-like (31). A plunger (31) may be movably positioned through a port (18a,b,c) of the top closure (17). In some embodiments, a top closure (17) may comprise a plurality of plungers (31) vertically movable (or actionable) through a plurality of ports (18a,bc) arranged in a top closure (17). For example, at least two, or three, or four vertically movable (or actionable) plungers may be positioned in a top closure (17). Each plunger (31) is movably positioned through a port (18) of the top closure (17). When a plurality of movable plungers (31) is present, the plungers (31) may be moved simultaneously or sequentially or randomly. The top closure (17) may further comprise a port (18c) connected to a microfluidic tube (24).

In some embodiments a movable (or actionable) protruding element (29, 31, 30) may be operated manually, mechanically, or magnetically. For example, a protruding element may be or comprise a metallic element movable in a magnetic field.

In some embodiments, a microfluidic device (1) may comprise at least bead, for example a metallic, positioned on the first surface (9) of a porous member (3), the bead(s) being intended to exert a mechanical constraint or a mechanical pressure on the surface (9) of the porous member (3) or in the volume of the porous member (3). The beads may be moved either by tilting up and down the microfluidic device (1) or by placing the microfluidic device (1) in an alternating magnetic field (an using a bead made of material sensible to the magnetic field).

In one of its objects, the present disclosure relates to a movable closure (33), suitable for a microfluidic device (1) as disclosed herein, the movable closure (33) being arranged to extend at least partly within the chamber (2) through the first zone (4), the movable closure (33) comprising an outward face (34) and an inward face (35), the inward face (35) being arranged to extend at the interface of the first and second zones (4,5).

In some embodiments, the inward face (35) may comprise at least partly an imprinting face (36). The inward face (35) may be arranged to be an imprinting face (36). The inward face (35) may be arranged to be contacted with the first surface (9) of a porous member (3). The imprinting face may comprise a plurality of reliefs (27).

The imprinting face (35) may comprise a plurality of reliefs (27), for example as above detailed.

In some embodiments, the imprinted face (35) may comprise regularly spaced reliefs. The regularly spaced relief may be cylindrical reliefs. The cylindrical reliefs may be comprised of cylindrical having a height of about 400 μm, a diameter of about 50-100 μm, and being spaced from each other of about 400 μm. The imprinted face (35) may delimit protruding structures defining an inverted pattern intended to be imprinted or embossed on the first or second surface (9, 10) of the porous member (3). In some embodiments, the pattern is intended to be imprinted or embossed on the first surface (9) of the porous member (3).

In some embodiment, the inward face (35) of the closure (33) may be surface treated. A treatment of surface may be selected to advantageously to reduce adherence or friction between the inward face (35) of the closure and the first face (9) of the porous member (3). A surface treatment may be selected to allow imprinting of the reliefs in the first surface (9) of the porous member (3) and prevent adhesion of the first surface (9) to the inward face (35) so as to avoid a pullout of the first surface (9) of the porous member (3) when the closure (33) is withdrawn. For example, the treatment of surface may be as described in the Examples section.

### Connections to Reservoirs or Instruments

A microfluidic device (1) or a microphysiological system as disclosed herein may be connected to any type of instruments or reservoirs.

As illustrated on FIG. 5, 6, 7A, 7B, 8, 9 or 10, in some embodiments, the lateral walls (6, 7) of the frame (32) and, optionally, the top and bottom closures (8, 17) may comprise at least one port (11, 12, 13, 14, 18). In some embodiments, the lateral walls (6, 7) of the frame (32) and, optionally the top and bottom closures (8, 17) may comprise at least one set of at least two ports.

In some embodiments, at least one port of the at least a first and/or a second sets of ports (11, 12, 13, 14) may be connected to a microfluidic tube (22a,b, 23a,b).

In some embodiments, at least two ports of the at least a first and/or a second sets of ports (11, 12, 13, 14) may be each connected to a microfluidic tube (22a,b, 23a,b).

In some embodiments, at least one port (18) the top and/or bottom closure (8, 17) may be connected to a microfluidic tube (24).

In some embodiments, the lateral walls (6, 7) of the frame (32) may comprise at least two set of at least two ports (11, 12, 13, 14). A first set of at least two ports (11, 12) may be arranged in the frame (32) to be in fluid communication with the first zone (4) and allowing a circulation of a fluid from a first port to a second port. A second set of at least two ports (13, 14) may be arranged in the frame (32) to be in fluid communication with the second zone (5) and allowing a circulation of a fluid from a first port to a second port.

The first and/or the second set(s) of at least two ports (11, 12, 13, 14) may comprise at least one, or more, ports. The additional port(s) may be un fluid communication with the chamber (2) or may be used to connect with, or insert in, an instrument and the inside of the chamber (2).

In some embodiments, at least one port (11, 12, 13, 14, 18, 20, 21) of a microfluidic device may be connected with a microfluidic tube (22a,b, 23a,b, 24a,b).

As shown on FIG. 10, in some embodiments, at least one of the ports (11, 12, 13, 14) within the lateral walls (6, 7) of the frame (32), and, if present, one of the ports (18) of the closure(s) (8, 17) may be connected with a microfluidic tube (22a,b, 23a,b, 24a,b).

In some embodiments, each of the ports (11, 12, 13, 14) within the lateral walls (6, 7) of the frame (32), and, if present, of the closure(s) (8, 17) is connected with a microfluidic tube (22a,b, 23a,b, 24a,b).

Suitable microfluidic tube (22a,b, 23a,b, 24a,b) may be made of polyethylene (PE) microfluidic tubes. Microfluidic tubes (22a,b, 23a,b, 24a,b) are sealed to the frame (32) and/or the closure(s) (8, 17) to prevent any leakage of materials or fluids from the chamber or contamination with material exterior to the chamber.

Microfluidic tubes (22a,b, 23a,b, 24a,b) allow connecting the inside of chamber (2) of microfluidic device (1) with reservoir or instruments.

In some embodiments, at least one port of the at least first and/or second sets of ports (11, 12, 13, 14) may be connected to a sensor or an actuator.

In some embodiments, at least two ports of the at least irst and/or second sets of ports (11, 12, 13, 14) may be each connected to a microfluidic tube (22a,b, 23a,b, 24a,b) and at least one additional port of the at least a first and/or a second sets of ports and/or at least one port the top and/or bottom closure (8, 17) may be connected to a sensor or an actuator.

In some embodiments, the microfluidic tube (22a,b, 23a,b, 24a,b) allows connecting the first and/or second zone(s) (4,5) with reservoirs for feeding the chamber (2) of the microfluidic device (1) with suitable media for cell culture, gas, solutions containing drugs to be tested, and for extracting from the chamber (2), exhausted media or samples of media or gas or solutions for further analysis.

In some embodiments, instruments for physico-chemical analysis, such as pH, pO₂, metabolites, such as lactate, pressure, shear stress, may be connected either to the ports present in the lateral walls (6, 7) of the frame (32) or in the closure(s) (8, 17).

In some embodiments, the ports (11, 12, 13, 14, 18) and/or the microfluidic tubes (22a,b, 23a,b, 24a,b) connected to the ports may be used to inject liquid or gas to modulate, for example, increase, the pressure inside the chamber (2) of the microfluidic device (1).

In some embodiments, the microfluidic device (1) can be connected to pumps, valves (e.g., rotary or pneumatic), bubble traps, oxygenators, gas-exchangers (e.g., to remove carbon dioxide), and in-line microanalytical functions. The microfluidic device (1) therefore can provide perfusion control and real-time metabolic sensing functions (e.g., O₂, pH, glucose, lactate), as well as feedback control capabilities as required to adjust the physical and chemical conditions of cell cultures.

Suitable valves may be of any kind known in the art, such as pinch valve, solenoid valve, manual, electric. At least one of the valves may fluidically connected or fluidically connectable with at least one compartment of the devices.

A pump may comprise a protruding microfluidic tube open or connected on one side to a fluid source, for example dipped in a reservoir containing a cell culture media or connected to a syringe, and connected to the other side to a microfluidic tube connected to one port (11, 12, 13, 14, 18) of the chamber (2). A suitable pump may be any type know in the art, such as peristaltic pump, syringe pump, pressure pump, gear pump, piston pump, centrifugal pump, electroosmotic pump, or vibrating pump.

In some embodiments, at least one of the microfluidic tubes (22a,b, 23a,b, 24a,b) is connected with a fluid control element. A fluid control element may modulate a fluid flow from inside to outside the chamber (2) or from outside to inside the chamber (2). For instance, a pump or a valve microchannel may be used to control the fluid circulation through the chamber (2), between microfluidic devices mounted in series or in in parallel, or for sample extraction from the chamber for analysis.

In some embodiments, at least one of the microfluidic tubes (22a,b, 23a,b, 24a,b) is in fluid connection with reservoir for collecting fluids exiting the chamber (2) or for feeding the chamber with suitable media, such as buffer or cell culture media.

In some embodiments, a microfluidic tube (24) connected to a port (18) in the top closure (17) may be used to feed oxygen into the chamber (2) for ensuring suitable cell culture conditions.

In other embodiments, a microfluidic tube (24) connected to a port (18) in the top closure (17) may be used to extract gas, such CO₂, from the chamber (2) for ensuring suitable cell culture conditions. Alternatively, a microfluidic tube (24) connected to a port (18) in the top closure (17) may be used to extract gas for analysis.

Microfluidic tubes (22a,b, 23a,b, 24a,b) connected to the ports (11, 12, 13, 14, 18) of the lateral walls (6, 7) of the frame (32) or of the top or bottom closures (8, 17) may be used to circulate a cell culture medium and/or a medium for collecting metabolites from the cell culture accessible either from the first zone (4) or from the second zone (5), for example by diffusion of the metabolites or of the nutriments from the culture medium through the porous member positioned in the chamber

In some embodiments, a microfluidic tube connected to a port may further be connected to a means for monitoring an environmental variable and/or a response of cells cultured in the chamber (2) of a microfluidic device (1) to a change of the conditions of the cell culture.

In some embodiments, a port (11, 12, 13, 14, 18) may be connected with at least one optical assembly. An optical assembly may allow for observations by optical imaging or detection means, or photostimulation, inside the chamber or a compartment. Optical imaging may include fluorescence, luminescence, photostimulation, light absorption, spectroscopy, chemiluminescence, electrochemiluminescence, optically activated chemical or biological reactions, such as light-induced transconformation, uncaging, polymerization, degradation, optogenetics, electro-optic surface modifications, and the like.

In some embodiments, a port (11, 12, 13, 14, 18) may be connected with at least one electrically conducting elements in electric connectivity with the inside of the chamber (2), for example within the first and/or the second zone.

A conducting element may comprise, or be part of, or be connected to, any electronic device, or any device involving the measure of a current, or the measure of a potential. As a non-exhaustive list of examples, said conducting elements may comprise pH sensors, ion-sensitive sensors, biosensors, chemical sensors, electrochemically active electrodes, piezoelectric elements, deformation sensors, position sensors, components of an impedancemetric sensor, temperature sensors, field effect transistors, and the like.

In some embodiments, the electrically conducting element may be a heating element.

In some embodiments, the electrically conducting element may be a solenoid, or a spire, generating a magnetic field.

In some embodiments, a port (11, 12, 13, 14, 18) may be connected with at least a magnetic element. A magnetic element may be a wire of a magnetic material, a magnetic core of soft magnetic material, or a magnet.

In some embodiments, a port (11, 12, 13, 14, 18) may be connected with at least one mechanical component, for example a plunger (31) or a movable blade (30) as above detailed.

### Uses and Methods of Use

### Cell Culture

In one of its objects, the present disclosure relates to a use of a microfluidic device (1) as disclosed herein, for cell culture.

A microfluidic device (1) as disclosed herein comprising cultured cells may be referred to as a microphysiological system (MPS).

In one of its objects, the present disclosure relates to a microphysiological system comprising at least a microfluidic device (1) as disclosed herein and at least one cell type cultured in suitable conditions in the first and/or second zone (4, 5) of said microfluidic device (1).

In some embodiments, the microphysiological system may comprise at least one cell type cultured in suitable conditions in suspension in the first and/or in the second zones (4, 5).

A microphysiological system may comprise at least a microfluidic device (1) as disclosed herein and at least one cell type cultured in suitable conditions in a zone (4,5) of said microfluidic device (1).

In some embodiments, the at least one cell type may be cultured in suitable conditions on the first surface (9) of the first porous member (3).

In some embodiments, the at least one cell type cultured in suitable conditions on the inner face of the microchannel (15) extended within the porous member (3).

In some embodiments, the at least one cell type may be cultured in suitable conditions within the porous member (3).

The cell culture may be cells cultured in suspension in the first zone (4), and/or cells cultured on the first surface (9) of the porous member (3), and/or on the internal face of a microchannel (15) in a porous member (3), or in a cavity delimited by the second surface (10) of a porous member (3) and the frame (32).

Cultured cells may be adherent or non-adherent cells. In some embodiments, cultured cells are adherent cells.

A microphysiological system may comprise at least one cell type cultured in suitable conditions in suspension in the first and/or in the second zones (4, 5).

A microphysiological system may comprise at least one cell type cultured in suitable conditions on the first surface (9) of the porous member (3).

A microphysiological system may comprise at least one cell type cultured in suitable conditions on a surface of the inner face of a microchannel (15a,b) positioned in the porous member (3).

A microphysiological system may comprise at least one cell type cultured in suitable conditions within the porous member (3), for example inside a hydrogel matrix.

Cells culture cultured on a surface of a given support, for example a first surface (9) of a porous member (3) are usually adherent cells.

Cells that may be cultured in microfluidic device (1) as disclosed herein may be any cells known in the art of cell culture.

Cells may be eukaryotes or prokaryotes. Cells may be bacteria, algae, yeasts, or mammal cells.

A microphysiological system of the disclosure may also be used to culture cells intended to reproduce the physiological environment of viruses.

Cells may be adherent or non-adherent cells.

Cells may be primary cultured cells or established cell lines, from human or animal tissue. Primary cultured cells may be obtained from biopsies, explants, or tissues samples.

The cells may be isolated from a tissue or a fluid using any methods known in the art, or differentiated from stems cells, e.g., embryonic stem cells, or induced pluripotent stem cells (iPSC), or directly differentiated from somatic cells.

Alternatively, the cells may be obtained from commercial sources, e.g., Cellular Dynamics International, Axiogenesis, Gigacyte, Biopredic, InVitrogen, Lonza, Clonetics, CD I, and Millipore, etc.).

In some embodiments, the cells may be from the “established” cell lines, e.g., A549, CaCo2, HT29, etc.

Suitable cells may be endothelial cells, alveolar epithelial cells, heart muscle cells, hepatocytes, Kupffer cells, airway smooth muscle cells, Sertoli and Leydig cells, astrocytes, fibroblasts, mucus cells, glandular cells, basal and goblet cells, cells of collecting tubules, proximal and distal tubular cells, cells of the epidermis, dermal endothelial cells, melanocytes, cells of the sweat glands, and cells of the hair root, follicular epithelial cells and parafollicular cells, hematopoietic stem cells, progenitor cells, reticular cells and blood cells (or precursors to blood cells) such as lymphocytes, monocytes, plasma cells and macrophages, zymogenic cells, centroacinar cells, and basal or basket cells and cells within the islets of Langerhans such as alpha and beta cells, stems cells, and/or induced pluripotent stem cells.

In some embodiments, the cells may be human CaCo2 cells.

In some embodiments, a microphysiological system may comprise at least one, or at least two, or at least three, or more, types of cultured cells.

In some embodiments, a microphysiological system may comprise co-cultured cells. Co-cultured cells may comprise at least two, or at least three, or more, types of cultured cells.

A microphysiological system of the disclosure may also be useable for the development and maintenance of small organism, such as zebra fish or Caenorhabditis elegans.

A microphysiological system may also be useable for the development and maintenance of biofilm produced by bacteria or yeasts.

The cultured cells may be seeded as suspension of cells or as pre-cultured organoids or spheroids. Spheroids are simple tissue or organ models that do not self-organize or have polarity and are thus not always representative of the more complex in vivo environment. Organoids are complex clusters of organ-specific cells designed to mimic the original tissue such as the skin, stomach, liver, or bladder.

The cells may be cultured in at least the first zone (4) of a microphysiological system as disclosed herein, either in suspension, on the first surface (9) of a porous member (3), and/or in the second zone (5), for example inside the porous member (3), in suspension in a microchannel (15a,b), as adherent cells on an internal face of a microchannel (15a,b), or in suspension in a cavity (19) delimited by the second surface (10) of the porous member (3) and the frame (32).

In some embodiments, the cells may be cultured inside the porous member (3), for example on reliefs present in said volume.

In some embodiments, the cells may be adherent cells cultured on the first surface (9) of a porous member (3).

In some embodiments, the cells may be cultured as adherent cells on reliefs (25) on the first surface (9) of the porous member (3).

The reliefs (25) may mimic colonic crypts of a colon, for example a human colon.

In some embodiments, a microphysiological system may comprise cells of a colonic tissue or human CaCo2 cells cultured reliefs (25) mimicking colonic crypts of a colon.

A culture media may comprise the nutrients and chemicals required for the maintenance and growth of the cultured cells.

Further a culture media may comprise chemicals or reactants for measuring or modulating the cell culture environment factors, such as pH, pO₂, CO₂, metabolites (lactate, . . . ). A cell culture media may contain markers, fluorescent or bioluminescent, for measuring cell growth, cell-cell interactions, cell-matrix interactions.

A cell culture may be supplemented with different factors suitable for modulating the environment of the cultured cells, such as microorganisms, such as bacteria or yeast; exosomes; proteins; growth factors, or nanoparticles.

### Applications

In one of its objects, the present disclosure relates to a use of a microfluidic device (1) or an MPS as disclosed herein, for cell culture.

In one of its objects, the present disclosure relates to a microphysiological system comprising at least a microfluidic device (1) as disclosed herein and at least one cell type cultured in suitable conditions.

The cells may be cultured in a zone (4,5), e.g., the first zone (4) of a microfluidic device (1), either in suspension or on the first surface (9) of the porous member (3).

In some embodiments, the cells may be cultured inside the porous member (3).

The cells may be cultured in a microfluidic device disclosed herein as above detailed. The cultured cells may be as above detailed.

A microphysiological system of the disclosure may be suitable for modelling and analyzing biological surfaces and biological tissue of a physiological interface. As example of biological surface or biological tissue of a physiological interface which can be modelled with a microfluidic device (1) as disclosed herein, one may mention the lining of the gastro-intestinal tractus, the lung, the skin, the mucosa, such as oral mucosa, vaginal mucosa, or ophthalmic mucosa.

In one of its objects, the present disclosure relates to a use of a microfluidic device (1) or an MPS as disclosed herein, for modelling a biological surface or a or biological tissue interface.

In one of its objects, the present disclosure relates to a method for culturing isolated cells, modelling a cell tissue, or modelling a biological tissue of a physiological interface, for example a colon tissue or a skin tissue, the method comprising at least the steps of:
providing at least one microfluidic device (1) as disclosed herein,
culturing, in suitable conditions, at least one cell type in at least one of at least first and/or second zones (4,5), the first and/or second zones (4,5) containing a cell culture medium in circulation through at least a set of at least two ports from the at least first set and/or second set of ports (11, 12, 13, 14).

In some embodiments, the cells may be cultured on the first surface (9) of the porous member (3).

In some embodiments, a first flux of a first cell culture medium is generated in the first zone (4) by circulating said first medium through at least a first set of at least two ports (11, 12) and/or a second flux of a second cell culture medium is generated in the second zone by circulating said second medium through at least a second set of at least two ports (13, 14), the at least two ports of the first and/or second set of ports (11, 12, 13, 14) being arranged for fluid circulation in the first and/or second zone (4,5).

In one of its objects, the present disclosure relates to a microphysiological system for modelling a biological tissue of a physiological interface, for example a tissue colon, the MPS comprising:
at least one microfluidic device (1) as disclosed herein,
wherein
the first zone (4) contains a cell culture medium in fluid communication with at least two ports (11, 12) of the at least first set of ports in the frame (32),
the microchannel (15) contains a physiologically acceptable medium in fluid communication with at least two ports (13, 14) of the at least second set of ports in the frame (32), and
at least one cell-type is cultured on the first surface (9) of the porous member (3).

In some embodiments, cells may be cultured on reliefs (25) present on a first surface (9) of the porous member (3).

In some embodiments, the reliefs (25) may represent crypt-like structures of a colonic tissue, and the cells may be cells from a colonic tissue, either primary cultured cells or established cell lines, for example human CaCo2 cells.

In some embodiments, a microphysiological system disclosed herein may be a model of a colon 64issue.

In some embodiments, a microphysiological system modeling a microenvironment of a colon may comprise a microfluidic device (1) comprising a two opposite walls (16a,b) and a frame (32) defining a chamber (2) comprising at least a first porous member (3) and at least a first (4) and a second (5) zones:
the frame (32) comprising at least a first (11, 12) and a second (13, 14) sets of ports, the first and second sets of ports comprising at least two ports (11, 12, 13, 14) arranged in the frame (32) for fluid circulation within, respectively, the first (4) and the second (5) zone,
the first porous member (3) extending partly in the chamber (2) and comprising a first surface (9) and a second surface (10) opposite to the first surface, the first surface (9) defining at least partly an interface between the first zone (4) and the second zone (5) of the chamber (2),
the first zone (4) being delimited in the chamber (2) at least between the first set of ports (11, 12) and the first surface (9) of the first porous member (3), and
the second zone (5) comprising the first porous member (3) and
   a microchannel (15) in the first porous member (3), the microchannel extending between the ports (13, 14) of the second set of ports, and/or
a portion of the chamber (2) delimited at least between the second set of ports (13, 14) and the second surface (10) of the porous member (3).

The chamber (2) of the microfluidic device (3) comprises a frame (32) which may comprise at least two lateral walls (6, 7) arranged between the opposite walls (16a,b).

A relief (25) may be present on the first surface (9) of the porous member (3).

The relief (25) may represent crypt-like structures.

At least one colonic cell type may be cultured on the reliefs (25) present on the first surface (9) of the porous member (3).

The first zone (4) may delimit a luminal compartment of a colon-like system.

The ports (11, 12) of the first zone (4) may be used to circulate a medium and control the environment of the luminal compartment.

The second zone (5) may delimit a stromal compartment of a colon-like system. The ports (13, 14) of the second zone (5) may be used to circulate a medium and control the environment of the stromal compartment.

The circulated media may be a chemically defined media allowing to control input factors for modulating or modifying the environment of the first and second zones (4,5) and extract output factors from the first and second zones (4,5) for further analysis.

A microphysiological system modeling a microenvironment of a colon may be referred to as a colon-like system.

A colon-like system as disclosed herein may advantageously reproduce a crypt topography.

A colon-like system as disclosed herein may advantageously mimic a colon extracellular matrix stiffness.

A colon-like system as disclosed herein may advantageously reproduce a fibroblastic niche.

A colon-like system as disclosed herein may advantageously allow recovering cells secretions.

A colon-like system as disclosed herein may advantageously allow using imaging approaches.

In some embodiments, a microfluidic device (1) of the disclosure may comprise a porous member (3) comprising at least one microchannel (15). The microchannel (15) may be used to perfuse liquid in the porous member, as previously described, or may be used to circulate objects for imparting dynamic deformation to the porous member.

The circulating object may be of metallic composition. A metallic object may be actuated by placing the microfluidic device containing such object in an alternating magnetic field or in moving magnetic field. Alternating and moving magnetic field may be generated by any known methods.

for example, a magnetic field may be generated with a Neodymium magnet Q-40-40-20 (Supermagnet)

To obtain deformation of the porous member, the movable object has size, for example a diameter in case of a bead, slightly greater than the width or diameter of the microchannel so as to deform the porous member (3) while it is moved, but to not be blocked or alter the structural integrity of the microchannel (15) and of the porous member (3).

A suitable object to be circulated may be a metallic bead. Suitable metallic bead may be Beads of 0.5 to 1 mm diameter, made of Steel 420c.

More than one circulating object may be used. For example, two circulating metallic beads may be used.

In some embodiments, a movable object, such as a metallic bead, placed in the microchannel (15) of the porous member (3) may be used to impart deformation to the porous member mimicking physiological or pathological deformation of a biological tissue, such as intestinal peristalsis.

For example, to model intestinal peristalsis, suitable cells may be cultured in suitable condition on the first surface (9) of the porous member (3), with the first surface (9) being imprinted with crypt-like reliefs (25). A metallic bead positioned within the microchannel (15) of the porous member (3) is actuated with an alternating magnetic field or a moving magnetic field to move the metallic bead back and forth along the microchannel (15) to obtain waves of deformation mimicking peristalsis.

In one of its objects, the present disclosure relates to an assembly comprising at least two microfluidic devices (1a,b,c,d, . . . ) as disclosed herein or at least two microphysiological system as disclosed herein, the at least two microfluidic devices or at least two microphysiological system being connected in series or in parallel.

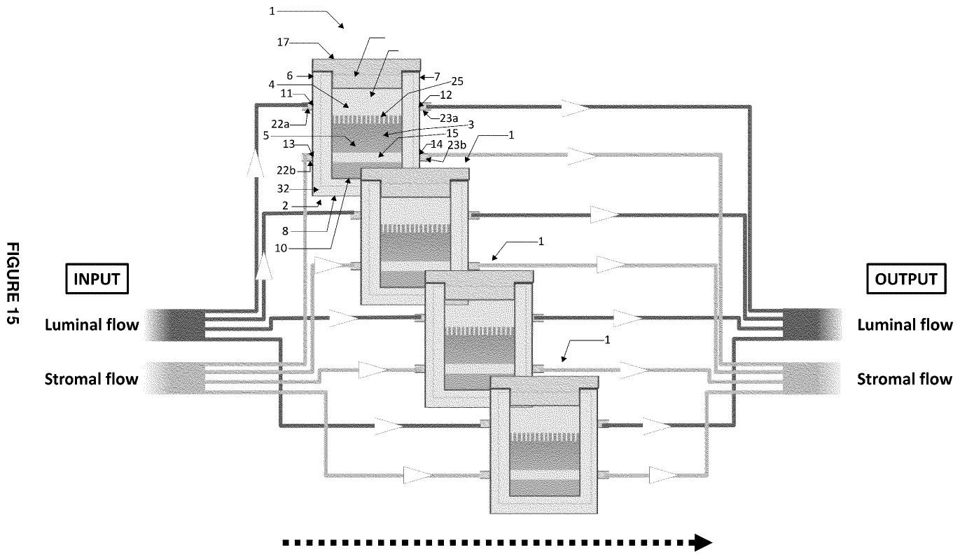

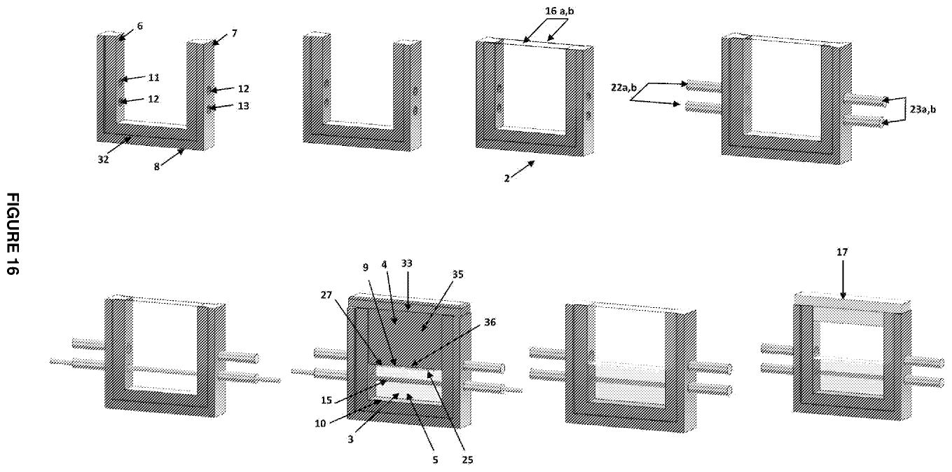

As shown in FIGS. 14 and 15, an assembly (26a,b) of the disclosure may comprise at least two, at least three, at least four, at least five, at least six, or more, microfluidic device (1a,b,c,d, . . . ) or microphysiological systems as disclosed herein connected in series (FIG. 14) or in connected in parallel (FIG. 15).

In some embodiments, in a configuration in series comprising at least two microfluidic devices (1ab), the first zone (4a) of a first microfluidic device (1a), or a first MPS (a), may be connected to the first zone (4b) of a second microfluidic device (4b), or a second MPS (b), by means of a microfluidic tube connecting a first port (12a), in fluid communication with the first zone (4a), and a first port (11b), in fluid communication with the first zone (4b). Also, or alternatively, the second zone (5a) of a first microfluidic device (1a), or a first MPS (a), may be connected to the second zone (5b) of a second microfluidic device (1b), or a second MPS (b), by means of a microfluidic tube connecting a second port (14a), in fluid communication with the second zone (4a), and a second port (13b), in fluid communication with the second zone (5b).

In some embodiments, in a configuration in parallel comprising at least two microfluidic devices (1a,b, . . . ), at least two ports (11a, 12a), in fluid communication with the first zone (4a), of the first set of at least two ports of a first microfluidic device (1a) are each connected with a microfluidic tube and at least two ports (11b, 12b) of the first set of at least two ports, in fluid communication with the first zone (4b), of a second microfluidic device (1b) are each connected with a microfluidic tube. A first series of microfluidic tubes connecting a first series of ports (11a,b, . . . ) may be connected to a same reservoir or to a principal tube connected to a reservoir and a second series of microfluidic tubes connecting a second series of ports (12a,b, . . . ) may be connected to a same reservoir or to a principal tube connected to a reservoir. The configuration may be reproduced at the second zone level. In a configuration in parallel comprising at least two microfluidic devices (1a,b, . . . ), at least two ports (13a, 14a), in fluid communication with the second zone (5a), of the first set of at least two ports of a first microfluidic device (1a) are each connected with a microfluidic tube and at least two ports (13b, 14b) of the first set of at least two ports, in fluid communication with the second zone (5b), of a second microfluidic device (1b) are each connected with a microfluidic tube. A first series of microfluidic tubes connecting a first series of ports (11a,b, . . . ) may be connected to a same reservoir or to a principal tube connected to a reservoir and a second series of microfluidic tubes connecting a second series of ports (12a,b, . . . ) may be connected to a same reservoir or to a principal tube connected to a reservoir

In some embodiments, in a configuration in parallel comprising at least two microfluidic devices (1ab), a first microfluidic tube comprising at least two branches, e.g., a Y-microfluidic tube, may be connected by a first branch (a) to a first port (11a), in fluid communication with a first zone (4a) of a first microfluidic device (1a), or a first MPS (a), and by a second branch (b) to a first port (11b), in fluid communication with a first zone (4b) of a second microfluidic device (1b), or a second MPS (b), A second microfluidic tube comprising at least two branches, e.g., a Y-microfluidic tube, may be connected by a first branch (a) to a second port (12a), in fluid communication with the first zone (4a) of the first microfluidic device (1a), or a first MPS (a), and by a second branch (b) to a second port (12b), in fluid communication with the first zone (4b) of a second microfluidic device (1b), or a second MPS (b). The first and second ports (11, 12a) and the first and second ports (11, 12b) are arranged for a fluid circulation in the first zone (4a) and in the first zone (4b), respectively. By multiplying the branches of the first and second microfluidic tubes it may be possible to add further microfluidic device or MPS. Also, or alternatively, the configuration may be repeated with the second zones (5). A first microfluidic tube comprising at least two branches, e.g., a Y-microfluidic tube, may be connected by a first branch (a) to a first port (13a), in fluid communication with a second zone (5a) of a first microfluidic device (1a), or a first MPS (a), and by a second branch (b) to a first port (13b), in fluid communication with a second zone (5b) of a second microfluidic device (1b), or a second MPS (b), A second microfluidic tube comprising at least two branches, e.g., a Y-microfluidic tube, may be connected by a first branch (a) to a second port (14a), in fluid communication with the second zone (5a) of the first microfluidic device (1a), or a first MPS (a), and by a second branch (b) to a second port (14b), in fluid communication with the second zone (5b) of a second microfluidic device (1b), or a second MPS (b). The first and second ports (13, 14a) and the first and second ports (13, 14b) are arranged for a fluid circulation in the second zone (5a) and in the second zone (5b), respectively.

The reservoirs connecting the first zones (4) and the second zones (5) may be the same or may be different. For example, they may be different. The reservoirs may contain different media or buffers for generating different environments in the first zones and in the second zones, for example, for mimicking a luminal compartment (first zones) and a stromal (second zones) compartment.

As detailed, in some embodiments, at least one of the ports arranged in the frame (32) of a microphysiological system of an assembly as disclosed herein, and optionally, or at least one of the ports of the closure(s) may be connected with a microfluidic tube. The microfluidic tubes allow connecting the microphysiological systems with reservoirs or instruments. The connection, reservoirs, and instruments may be as above detailed.

A set of microphysiological systems connected in series or in parallel may be used to model the different compartments of an organ.

When assembled in series, a set of microphysiological systems as disclosed herein may be used model the different organs of a given tractus. For example, a set of microphysiological systems may be assembled in series to model the gastro-intestinal tractus with one microphysiological system representing the oral cavity, one microphysiological system representing the esophagus, one microphysiological system representing the stomach, one microphysiological system representing small intestine, one microphysiological system representing the large intestine, one microphysiological system representing the colon, and one microphysiological system representing the rectum.

A microphysiological system as disclosed herein may be used for modelling and analysis cell-cell interactions, cell-extracellular matrix (ECM) interactions, cell-biological fluid interactions, cell migration, cell invasion, chemotaxis, patterning effect, bioprinting, or injection effect.

A microphysiological system as disclosed herein may be used in pre-clinical studies for modelling and analysis cancer conditions, diabetes, physiopathological metabolism, irritable bowel disease, effect of the nutrition on the microbiome of the gastro-intestinal tractus, etc.

A microphysiological system as disclosed herein may be used as imaging chamber with controlled and conditioned environment. Thanks to the lateral walls as above detailed, a microphysiological system, or a microfluidic device, as disclosed herein may be used with any suitable microscopy instrument, such as broad field microscopy, confocal microscopy, bi-photonic microscopy, multiphoton microscopy, selective plane illumination microscopy (SPIM).

### Manufacturing Methods

In one of its objects, the present disclosure relates to a method for manufacturing a microfluidic device (1) as disclosed herein.

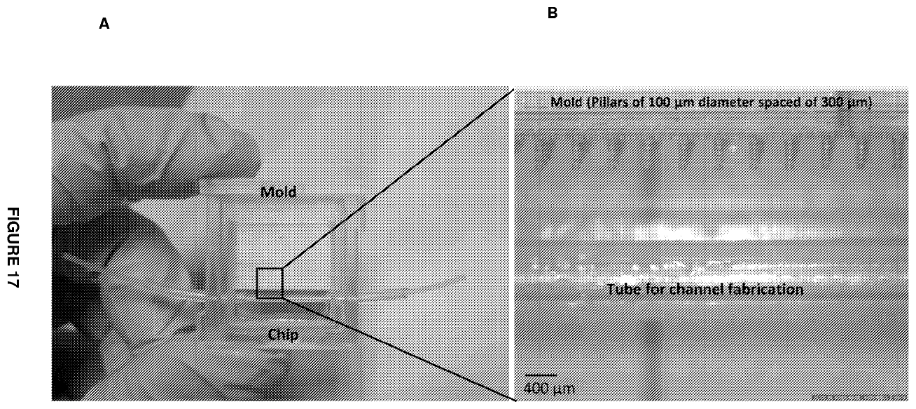

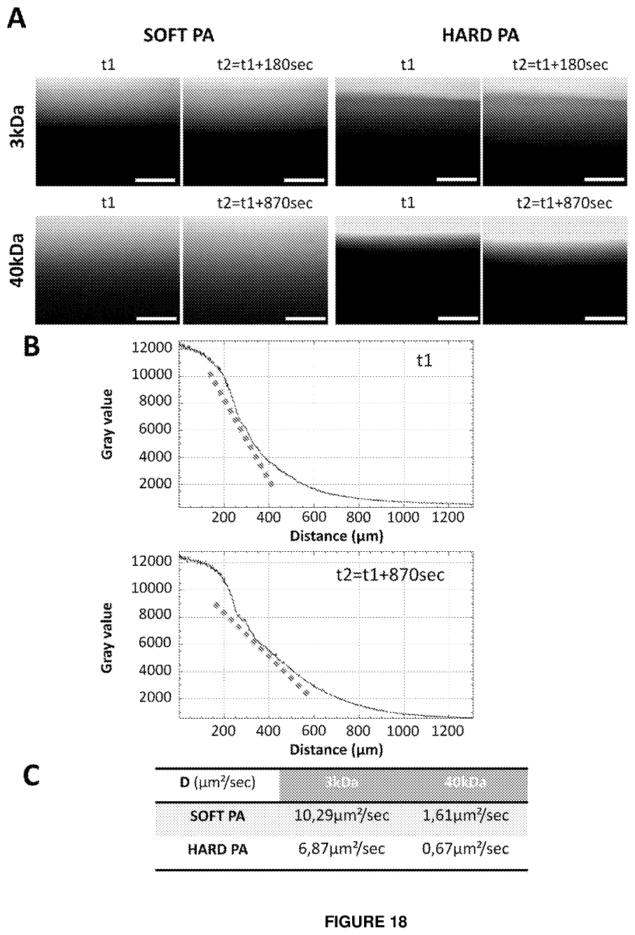

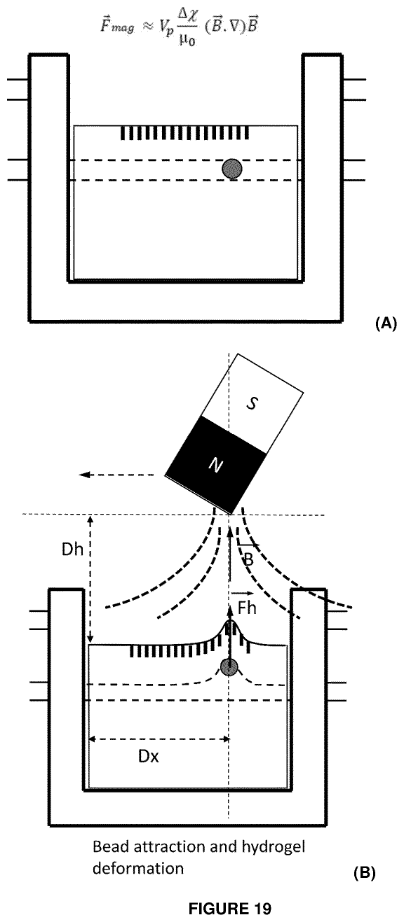

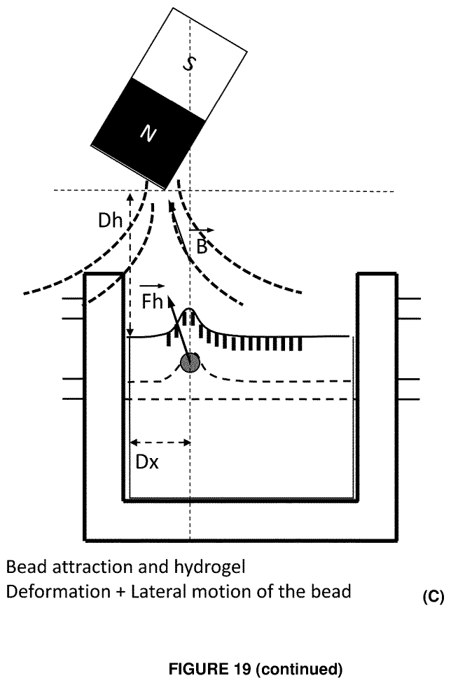

FIG. 16 illustrates the steps of the manufacture of a microfluidic device (1) disclosed herein.

A method of the disclosure may comprise at least the steps of:
providing a chamber (2) comprising two opposite walls (16a,b) and a frame (32), the frame comprising at least a first (11, 12) and a second (13, 14) sets of ports, each of the first and second sets of ports comprising at least two ports arranged in the frame for fluid circulation within the chamber (2),
forming at least a first porous member (3) in the chamber (2), for obtaining at least a first and a second zones (4,5), the first porous member (3) comprising a first surface (9) defining at least partly an interface between the first and second zones (4,5) and a second surface (10) opposite to the first surface,
the first zone (4) being delimited in the chamber (2) at least being between the first set of ports (11, 12) and the first surface (9) of the first porous member (3), and
the second zone (5) comprising the first porous member (3) and
a microchannel (15) in the first porous member (3), the microchannel extending between the ports (13, 14) of the second set of ports, and/or
a portion of the chamber (2) delimited at least between the second set of ports (13, 14) and the second surface of the porous member (10).

The present disclosure relates to a method for manufacturing a microfluidic device (1) as disclosed herein, the method comprising at least the steps of:
providing a chamber (2) delimited by a frame (32) and two opposite walls (16a,b),
forming a porous member (3) in the chamber (2), the porous member (3) comprising a first surface (9) and a second surface (10), the first surface (9) separating the chamber (2) in a first and a second zones (4,5), wherein
the frame (32) comprises at least a first and a second sets of ports (11, 12, 13, 14), the first set of ports (11, 12) comprising at least two ports arranged in the frame (32) for fluid circulation within the first zone (4) and the second set of ports comprising at least two ports (13, 14) arranged in the frame (32) for fluid circulation within the second zone (5), and the two ports (13, 14) of the second set of ports being open
(i) a microchannel (15) extending through the porous member (3), or
(ii) in a cavity (19) arranged between the second surface (10) of the porous member (3) and the frame (32) of the chamber (2).

A microchannel (15) in the first porous member (3) may be obtained by:
positioning within the chamber (2) at least one elongated member extending through the ports (13, 14) of the second set of ports,
casting in the chamber (2) at least one material suitable to embed said elongated member and to form a porous member (3).

Further to the casting of the material suitable to embed the elongated member, the material may undergo a step of curing, polymerization, or hardening to form a porous member.

After obtaining the porous member, the elongated member may be removed to obtain a microchannel (15) in the porous member (3). The elongated member may have any size and shape provided that it can be extended through ports of the second set of ports and may be withdrawing after obtaining the porous member.

For example, an elongated member may be a tube, a wire, a thread.

In some embodiments, a porous member may comprise a network of microchannels. A network of microchannels may be obtained either by positioning an elongated member with multiple branches or multiple elongated members.

In some embodiments, an elongated member with multiple branches may be a sugar-based elongated member which may be dissolved in water after obtaining the porous member.

In some embodiments, the porous member may be a hydrogel matrix. For example as above disclosed.

A material suitable to form a hydrogel matrix may be as above disclosed. For example, it may be carboxymethyl cellulose, a modified starch, alginate, pectin, hyaluronic acid, collagen, matrigel, decellularized matrix, gelatin, polyacrylamide, polyethylene glycol, hydrogel of poly (2-hydroxyethyl methacrylate) (pHEMA), GELMA, PDMS, Nylon, Matrigel, Cultrex, and combination thereof.

A cavity (19) may be obtained may be obtained by any suitable methods.

To obtain a cavity (19), it may be useful to use for the chamber (2), a frame (32) comprising a movable bottom closure (8).

For example, a cavity (19) may be obtained by forming a porous member (3) maintaining an empty space in a part of the second zone (5) of the chamber (2), this part being positioned between the second surface (10) of this porous member and the frame (32). To obtain such empty space, one may use a bottom closure (8) with an inward face protruding inside the chamber (2), within a part of the second zone (5), at the time of forming of the porous member (3) inside the chamber (2). Once the porous member (3) is formed, the bottom closure with an inward face protruding inside the chamber (2) is replaced with a bottom closure (8) with an inward face not protruding inside the chamber (2) or protruding with a less extent compared to the bottom closure with a protruding inward face, so that an empty space is obtained between the second surface (10) of the porous member and the frame (32).

Alternatively, a cavity (19) may be obtained by first placing a movable item intend to partially filled the zone of the chamber (2) intended to be the second zone (5), casting the material suitable for obtaining the porous member (3), opening the bottom of the frame (32) by removing the bottom closure (8) so as to remove the previously placed item, and then closing the bottom of the frame (32) with the bottom closure (8).

The step of providing the chamber may comprise a step of bonding two opposite walls (16a,b) to a frame (32), as disclosed herein.

In some embodiments, a manufacturing method may further comprise a step of connecting, at least, a first microfluidic tube to a first port (11 and/or 13) and a second microfluidic tube to a second port (12 and/or 14), the first and second ports (11, 12 and/or 13, 14) being from the at least first or second sets of ports and being arranged in the frame for fluid circulation within the first or the second zone.

In some embodiments, the manufacturing method may further comprise a step of forming reliefs on the first surface (9) of the porous member (3) by contacting the first surface with a movable closure (33) for imprinting as disclosed herein. Such a with a movable closure (33) may comprise an outward face and an inward face, the inward face being an imprinting face arranged to extend and contact with the first surface (9) of the porous member (3) and comprising a plurality of reliefs. The reliefs may be, as disclosed herein, the inverted reliefs to be imprinted.

In some embodiments, reliefs may be provided within a volume of the porous member.

In some embodiments, the opposite walls (16a,b) walls may be glued to the frame (32), for example to the lateral walls of the frame (32). Gluing may be implemented with any suitable gluing material known in the art. For example, a double-sided adhesive may be used.

In some embodiments, the opposite walls (16a,b) may be silanized glass walls Silanization may be carried out according the APTES/glutaraldehyde protocol described by Seed B. in Curr Protoc Mol Biol. 2001.

In some embodiment, a frame (32) may be obtained by molding or 3D printing. For example, a frame (32) may be printed by stereolithography.

In some embodiments, the manufacturing method may further comprise a step of connecting a microfluidic tube to at least two ports of at least the first (11, 12) and/or the second (13, 14) set of ports. Suitable microfluidic tubes may be as disclosed herein, for example they be microfluidics PTFE tubings. The microfluidic tubes may be hermetically sealed to the ports to prevent leakage of inwards contamination.

In some embodiments, the manufacturing method may further comprise a step of imprinting reliefs (25) on the first surface (9) of the porous member (3).

The imprinting or embossing of a pattern may be carried by a mold, or a movable closure (33) for imprinting as disclosed herein.

In some embodiments, the reliefs may delimit an inverted crypt-like pattern.

A movable closure (8, 17, 33) may be obtained by molding or 3D printing. In some embodiments, a movable closure (8, 17, 33) may be printed by stereolithography.

In some embodiments, the inward face comprising at least partly an imprinting face of a movable closure (33) for imprinting may further be chemically treated.

A chemical treatment may be carried to improve the contact between imprinting face and the first surface of the porous member, and improve the imprinting or the embossing of the reliefs.

As example of chemical treatment, one may cite treatment with an organic solvent, such as acetone, self-assembled monolayer grafting in a gaseous phase with SiO₂, and FDTS (1H,1H,2H,2H-Perfluorodecyltrichlorosilane).

In one of its objects, the present disclosure relates to a method for manufacturing a microphysiological system as disclosed herein. The method comprises the steps of manufacturing a microfluidic device (1) as disclosed herein and further a step of culturing, in suitable conditions, cells in the first and/or second zones (4,5), for example as disclosed herein, and for example on the first surface (9) of a porous member (3).

FIG. 17A represents a photography of a microfluidic device as disclosed herein. FIG. 17 B is a photography of reliefs, presenting a crypts-like structure imprinted on the first surface (9) of the porous member (3).

It is to be understood that the disclosure encompasses all variations, combinations, and permutations in which at least one limitation, element, clause, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The following examples are provided for purpose of illustration and not limitation.

### EXAMPLES

### Example 1: Materials & Methods

### Collagen Preparation

To prepare 1 mL of a neutralized collagen type 1 (Coll 1) solution (at a final concentration of 1 mg/mL), 265 μL of collagen (Corning 354236; rat tail collagen type 1, 3.77 mg/mL in 20 mmol/L acetic acid) were mixed with 6.1 μL of sodium hydroxide (NaOH; Sigma S2770; stock concentration of 1 mol/L, working concentration of 30 mmol/L), 20 μL of HEPES (Sigma 83264; stock concentration of 1 mol/L, working concentration of 20 mmol/L), 77.4 μL of sodium bicarbonate (NaHCO₃; Sigma S5761; stock concentration of 7.5% wt/vol, working concentration of 0.58% wt/vol), 100 μL of 10× phosphate-buffered saline (PBS; Sigma D1408; working concentration of 1×), and 531.2 μL of deionized water on ice. This solution was mixed by pipetting. To prepare the same collagen solution at final concentration 5 mg/mL, 455 μL of collagen (Corning 354249; rat tail collagen type 1, 10.98 mg/mL in 20 mmol/L acetic acid) were mixed with 10.4 μL NaOH, 20 μL HEPES, 132.6 μL NaHCO₃, 100 μL of 10×PBS and 282 μL of deionized water on ice.

The collagen was homogenized by pipetting and then left polymerized for 30 min. at 37° C.

To stain the collagen network, 0.3 μL AlexaFluor 647 NHS Ester (Life Technologies, A37573, stock concentration 0.1 mg/mL) was added to collagen solution prior reticulation.

### Polyacrylamide (PA) Hydrogel Preparation

To prepare 1 mL of PA solution, 250 μL, for hard PA gel, or 100 μL, for soft PA gel (Sigma A3553; stock concentration of 40%, working concentration of 10% for hard PA gel or 4% for soft PA gel) of acrylamide were mixed with 112 μL of bis-acrylamide for both rigidity gels (N,N′Methylenebisacrylamide; Sigma M7279; stock concentration of 2%, working concentration of 0.22%) and 627 μL of deionized water for hard gel or 777 μL for soft gel. For PA hydrogel staining, 10 μL of fluorescein dimethacrylate (Sigma 250249; stock concentration of 1 mg/mL, working concentration of 10 μg/mL) were added prior reticulation. Reticulation reaction does not start until addition of APS and TEMED, this is why theses catalyzers should be mix extemporaneously with PA: 10 μL APS (Ammonium persulfate; Sigma 248614; stock concentration of 10%, working concentration of 0.1%) and 1 μL TEMED (Tetramethylethylenediamine; Sigma T7024; working concentration of 0.1%) were added to acrylamide/bis-acrylamide mixture, then the preparation was vortexed and seeded in microdevice chamber.

Soft and Hard PA gels correspond respectively to a stiffness (Young's modulus value) of 4.03 kPa and 13.88 kPa (Measures obtained using atomic force microscopy (AFM)).

Depending on the experiments, different types of hydrogels were prepared: PA 10%+Collagen coating 1 mg/mL, PA 10%+Collagen coating 5 mg/mL, or PA 4%+Collagen coating 1 mg/mL.

### Atomic Force Microscopy (AFM)

AFM measurements were performed using Borosilicate Glass Particle (10 μm) on silicon nitride cantilevers (PT.BORO.SN.10, Novascan Technologies) with a nominal spring constant of 0.06 N/m mounted on a NanoWizard Ill AFM (JPK Instruments) coupled to an inverted optical microscope (Axiovert 200, Carl Zeiss).

To ensure reproducibility in force application and measurement, the cantilever sensitivity and spring constant were calibrated before each experiment using the JPK Instrument software using the thermal noise method. Force-distance curves were recorded in contact mode in liquid at 2 Hz (1 second per approach-retract force curve) with an applied force of 1 nN.

Each hydrogel was tested at 4 locations with about 250 force curves acquired per location and 512 data points per curve. The measured area was 150 μm×150 μm per location.

### 3D Design & Printing of the Microfluidic Device

A mold was designed to contain pillar patterns whose dimensions correspond to the average dimension of human colonic crypts (100 μm diameter and 400 μm height). These inverted patterns (pillars) allow obtaining invaginations when replicated into a hydrogel scaffold. The dimensions of the crypts patterns on the 3D mold have been validated (98 μm-diameter and 417 μm-height).

Briefly, the mold and chamber's window design (chamber's frame) of the microfluidic device were made with CAO software (Fusion 360). Each design was exported to a STL file. This file was printed by stereolithography using DWS29J+system and DS-00 biocompatible resin for DWS company (Italy). The design was sliced in 50 μm thick layers (except for colonic crypt-like structures on the mold which were sliced in 10 μm) and printed with a 17 μm spot size (405 nm) at 5800 mm/s speed.

After printing, chamber's window was removed from the building table, developed in a bath of ethanol for 10 min, and then post-polymerized in a UV chamber (32 mW) during 30 min. It was then post-polymerized in a UV chamber (32 mW) during 30 min

After printing and removing from building table, the mold was developed in a bath of acetone for 10 min and treated by SAM (Self Assembled Monolayer) grafting in a gaseous phase with SiO₂ (5 min) and FDTS (1H,1H,2H,2H-Perfluorodecyltrichlorosilane) (5 min). This post-development steps helps molding hydrogel with the inverted crypt-like structure.

The microfluidic device chamber's lid was made by PDMS (polydimethylsiloxane) molded on a 3D printed mold. The lid's mold was designed and printed as described above but with a slicing of 30 μm.

After printing, the 3D mold was developed by an acetone bath for 10 min, and then treated with the SAM method as described above, and then the PDMS chamber's lid was molded on it.

### Glass Silanization

To create covalent bonds between the polyacrylamide hydrogel and the glass coverslips of the microfluidic device, a glass coverslip treatment was performed according the APTES/glutaraldehyde protocol described by Seed B. in Curr Protoc Mol Biol. 2001.

Glass coverslips (20 mm×20 mm) were washed with acetone, ethanol and deionized water and then dried. Then, coverslips were treated with oxygen plasma for 5 minutes (100% generator power & 0.5 mbar pressure, Plasma Pico system, Diener GmbH). Plasma treated coverslip side was bathed in pure APTES ((3-Aminopropyl)triethoxysilane; Sigma, 440410) solution for 15 minutes at room temperature and then washed three times with deionized water. Coverslips were then bathed for at least 30 minutes (up to a maximum 1 hour) in glutaraldehyde 0.5% solution (Sigma, 340855 reconstituted in deionized water), protected from light and washed three times with 50 mL of deionized water before drying.

Silanized glass coverslips were stored on PARAFILM© at 4° C. in hermetic Petri dish up to 3 weeks until further use.

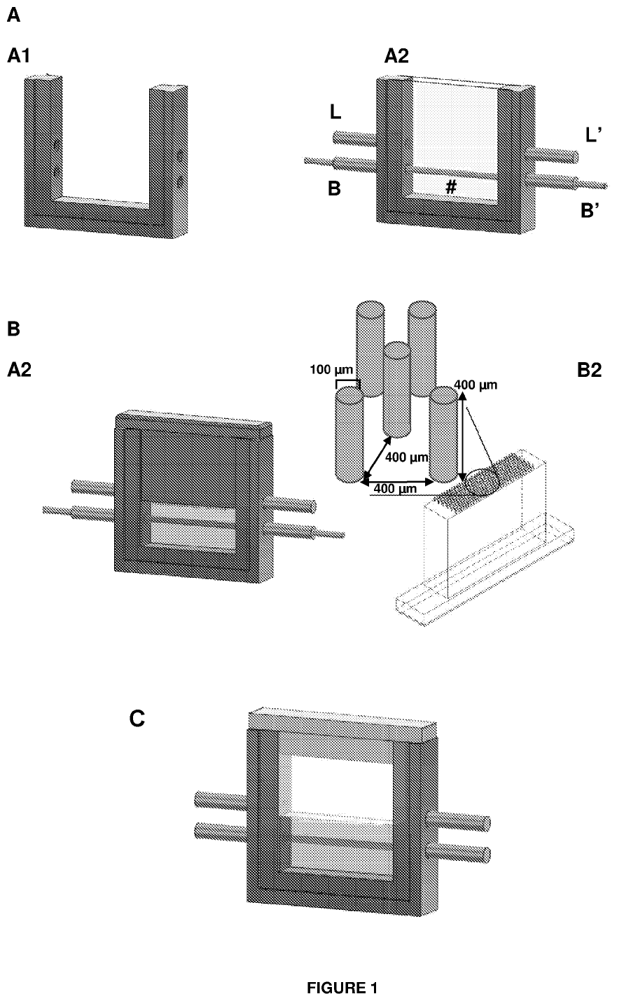

### Microphysiological System (MPS) Assembly—FIG. 1

Microfluidics PTFE tubings (Polytetrafluoroethylene, 1.6 mm diameter) were inserted in each inlet and outlet ports of the 3D printed microfluidic device (FIG. 1A). In order to prevent leakage, a drop of DS3000 photoresist was inserted at the tubing-MPS interface and exposed to UV light for 5 minutes. An internal tube (#) (900 μm diameter) passing though basal connectors (B, B′) was added to create basal fluidic microchannel and left out after PA hydrogel reticulation (FIG. 1A).

Double-sided adhesives (100 μm thickness) were pasted on each side of the frame of the chamber to bond the silanized glass coverslips (silanized face inwardly oriented) (FIG. 1A).

A volume of 600 μL of PA solution mixed with APS and TEMED as above-indicated was poured into the chamber of the device made with the U-shaped frame enclosed with the silanized glass coverslips (FIG. 1B).

The mold bearing pillar patterns was first pre-coated with 10 μL of collagen I, left incubated 30 min at 37° C., and then inserted into the microfluidic device to be brought in contact with the PA surface (FIG. 1B).

After 30 min of reticulation, as previously indicated, at room temperature, the mold was gently vertically removed, the internal tube (#) was withdrawn, and PBS was added above the PA hydrogel to fill in the remaining volume (luminal compartment) inside the MPS (FIG. 1C).

The device was stored in PBS at 4° C. until use.

### Glass Substrates Preparation

For molding characterization, a simplified model of the MPS was used.

Eighty μL of a solution collagen I at 1 or 5 mg/mL were deposited on a mold bearing inverted crypt-like structures and left polymerized for 30 minutes at 37° C. Then 100 μL of PA solution (for Soft or Hard PA hydrogel) were added to the mold coated with the polymerized collagen and a silanized glass coverslip was placed on the mold. After 30 minutes of crosslinking of the PA, the mold was removed and the coverslip containing the molded hydrogel was rinsed in PBS and stored at 4° C. until use.

The collagen I at 1 or 5 mg/mL and the PA solutions for Soft and Hard PA hydrogels and the silanized glass coverslips were produced as described in the previous paragraphs.

### Cell Culture

Cell culture experiments were performed the day following casting the PA hydrogels (10% or 4%) coated with Collagen 1 (1 mg/mL or 5 mg/mL) and embossing with mold bearing inverted crypt-like structures (at least an overnight incubation in PBS was carried out before cell culture). Before cells plating in the MPS, the latter was exposed to UV light (254 nm) for ensuring sterility and incubated for at least 1 hour at 37° C., 5% CO₂ in culture media.

Caco-2 cells (ATCC, htb-37) were cultivated in DMEM 4.5 g/L glucose (ThermoFischer, 31966-021) supplemented with 10% Fetal Bovine Serum (FBS, ThermoFischer, 10270), 1% non-essential amino acids (NEAA, ThermoFischer, 11140-050) and 2 μg/mL penicillin/streptomycin (ThermoFischer, 15140-022). Cells were plated at 10 000 cells per cm² into the MPS and the medium was renewed every 3 days.

### Immunofluorescence

Cells cultured in the MPS were rinsed with PBS and fixed with 3.7% PFA (paraformaldehyde, Sigma, 252549) for 15 min. Fixed-cells were permeabilized with PBS-0.1% Triton X-100 (Sigma, T8787) for 15 min, then washed three times in PBS (Eurobio, CS1PBSO1-01). Putative non-specific sites were blocked with PBS-5% SVF for 1 hour. Samples were incubated with primary antibodies ZO-1 (1:100, Invitrogen, 33-9100, Mouse) and K167 (1:100, Cell Signaling, 9129, Rabbit) diluted in PBS-5% SVF overnight à 4° C. After three washes with PBS, cells were incubated for 1 hour with secondary antibodies labeled with a fluorescent dye (1:500, AlexaFluor 488 goat anti-mouse (Invitrogen, A11001) and AlexaFluor 568 donkey anti-rabbit (Invitrogen, A10042)). For F-actin co-staining,phalloïdin 568 (1:100, Invitrogen, A12380) was added to secondary antibody solution. Cells were then rinsed three times in PBS, the second wash containing DAPI (1 μg/mL, Invitrogen, 62248) for nuclear DNA staining.

### Image Acquisition and Analysis

Brightfield images used for pillars structures profilometry on the mold were acquired with the 3D digital Hirox microscope (HRX-01). Brightfield imaging was performed along cell culture with a Nikon Eclipse Ts2 microscope (10× or 20× objectives) using a Nikon DS-Fi3 camera and DS-L4 control unit.

Confocal images for hydrogel characterization and after immunostaining were acquired on an upright Leica SP8 confocal microscope equipped with 2 PMT detectors, 488 nm, 552 nm and 638 nm diodes and a 25× physio objective.

Second harmonic generation microscopy was performed to confirm collagen fibrillar structure using an upright multiphoton Zeiss LSM 7 MP microscope equipped with a pulsed laser adjustable from 690 to 1080 nm and a 40× water immersion objective. Images were treated and analyzed with ImageJ 1.53.

### Statistical Analyses

Statistical analyses were performed, and graphs were generated using the GraphPad Prism 9.2 Software. Data are presented as mean±SD.

### Example 2: Results

### Replication of PA Crypt-Like Structures

First experiments were devoted to assessing the molding of colon-like structures into PA hydrogel materials casted directly onto the mold bearing the inverted crypt-like structure.

First the casting of both Soft and Hard PA materials deposited as a layer onto glass substrates and further embossed with a 3D printed mold was investigated. Soft and Hard PA gels correspond respectively to a stiffness of 4.03 kPa and 13.88 kPa (Measures obtained using atomic force microscopy (AFM)).

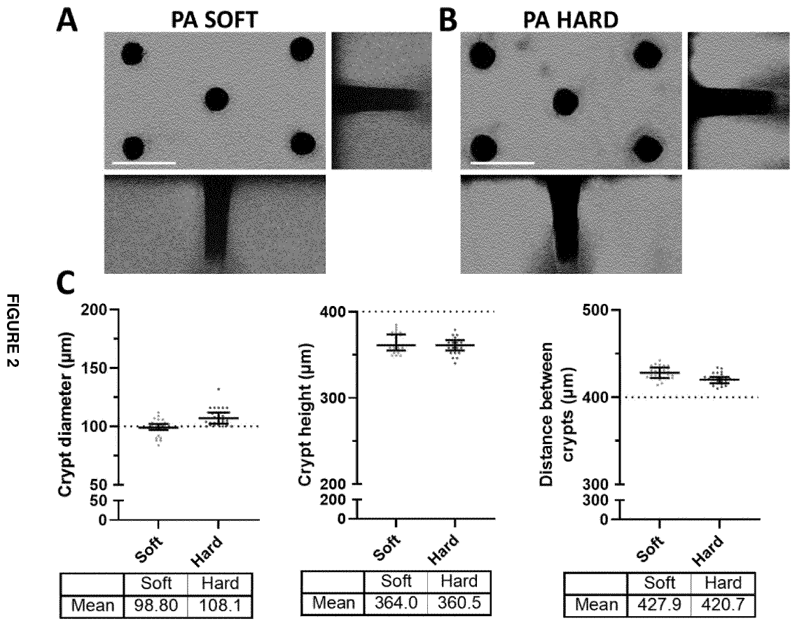

Preliminary experiments were performed by depositing the liquid PA formulation on the 3D printed mold, then covered with a glass slide, to replicate the pillar structures into the PA hydrogel. As shown in FIGS. 2A and 2B, both PA formulations, hard and soft, were successfully molded with a resolution that matches the expectations regarding the reproduction of crypt-like structures.

Dimensional analysis (FIG. 2C) of both height and diameter of the molded structures showed similar mean diameter values of about 98 μm and about 108 μm for soft and hard PA materials respectively. The mean height of the structures was determined at about 364 and about 360 μm, respectively. These values were in good agreement with the expected mold dimensions corresponding to average human colon crypt heights. They exhibited a 10% variation around the theoretical value which could result from the slight swelling that may be associated with the PA hydrogel reticulation process during molding. In line with these first observations, the swelling would likely induce a slight increase in the observed gap between pillars, determined respectively at about 427 and about 420 μm for the soft and hard materials. This result was also consistent with the 10% variation observed for the pattern dimensions.

### Replication of Collagen I/PA Human Colon-Like Topologies

From the establishment of this first proof of concept, the replication of crypt-like structures into dual Collagen I (Coll I)/PA hydrogel networks was further investigated.

Indeed, PA scaffolds are not compatible with cell culture as the PA gel cannot promote the adhesion of cells at its surface. An additional step is thus required to functionalize PA hydrogel with proteins allowing the cells adhesion on the scaffold. Here, Collagen I, a main component of colon extracellular matrix, was used. In vitro reticulation of Collagen I allowed to form fibrillar structures as observed in vivo in the tissues.

As mentioned above, molds bearing inverted crypt-like structures were coated with Collagen I solutions at 1 or 5 mg/mL prior to their insertion in the MPS chambers filled with PA solutions (Soft or Hard PA) in the following combinations: Soft PA+Coll I 1 mg/mL, Hard PA+Coll I 1 mg/mL, and Hard PA+5 mg/mL.

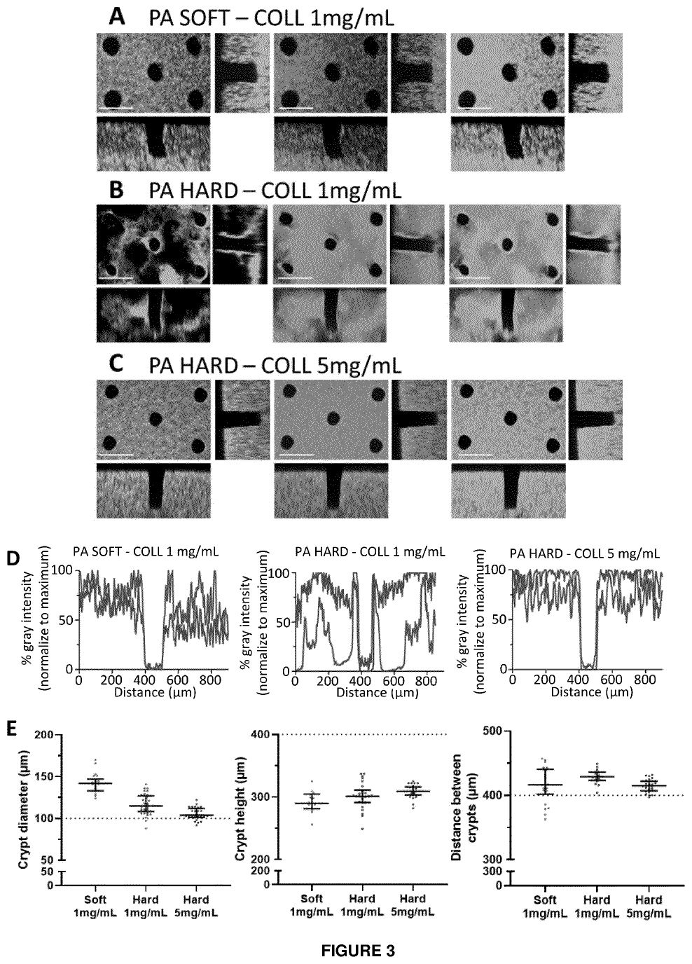

FIG. 3 (A-C) shows the morphology of the replicated structures. While regular arrays of pillars could be successfully obtained with the three conditions, the intensity profiles obtained from crosscut images show a clear difference in the spatial distribution of the materials (FIG. 3D). Hard PA combined with 5 mg/mL Collagen I exhibited very similar distribution, suggesting an intimately intricate network of both materials. While combination of Collagen I at 1 mg/mL with Soft PA gave comparable results, increasing the Collagen 1 at a concentration up to 5 mg/mL resulted in a less reproducible homogeneous structure (FIG. 3D).

A clear correlation was observed between the material composition and the structure replication accuracy (FIG. 3E). Soft PA/Collagen I (1 mg/mL) were providing the largest deviations of structure diameters and height as compared to the original mold dimensions, while combination of hard PA with Collagen I at 5 mg/mL provided better agreement of the replicated structure dimensions as compared to the mold design. To note, the three formulations provided lower structure height than PA only structures.

These observations suggest that the replication accuracy may be modulated by the composition and mechanical properties of the hydrogel material. It was first hypothesized that the addition of Collagen I on the mold favors the creation of an intricate network. This was confirmed by the image analysis of the material distribution that shows the co-localization of both material in the area of interest (FIG. 3D). It was believed that the replication accuracy is improved while material stiffness is increasing. It is known from literature (Joshi et al. Biotechnol Bioeng. 2018) that Collagen I stiffness, in a 1 to 5 mg/mL concentration range, exhibits Young's modulus values varying from 100 Pa to 1.4 kPa respectively. It can reasonably be estimated that the stiffness of Collagen I/PA hydrogel network is thus influenced by the properties of PA hydrogel, then giving rise to a scaffold material with rigidities below the ones of Collagen hydrogels. This observation is converging with the observed evolution of the structures' dimensions which suggest a better replication accuracy for highest PA (Hard PA) and/or Collagen I concentrations. This tendency was confirmed by the analysis of the structure diameter that shows best accuracy for Hard PA/Collagen I 5 mg/mL combination (FIG. 3E). This phenomenon matched as-well the evolution of the structure height even though a lower impact of the material composition on the structure height was noticed.

Colorectal Culture into the Human Colon-Like Microphysiological System

A crucial point of this study was the ability to establish cell culture conditions allowing to obtain an epithelium lining from the bottom of the crypt at the surface of the upper flat compartment.

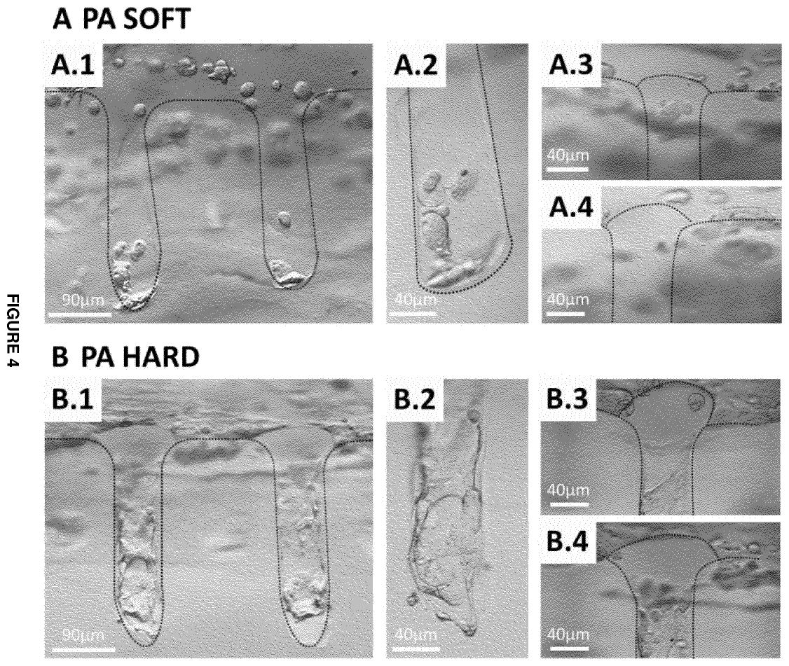

Caco-2 cells were thus plated into the MPS containing either a Soft or a Hard PA hydrogel combined with Coll I (1 mg/mL). The day after plating, cells were able to adhere on the hard PA gel but not on the soft one (FIG. 4). On hard PA hydrogel, epithelial cells were able to colonize both the crypts and the flat compartment of the scaffold forming a continuous epithelial monolayer, while the cells in the MPS containing the soft PA gel remained round, non-adherent and not being able to grow into a continuous epithelial layer (FIG. 4A1-A4 and FIG. 4B1-B4). Caco-2 cells cultures in the MPS could last up for at least 21 days. The culture was stopped at this stage in order to perform a characterization using immunofluorescence staining on the epithelial lining.

The cell culture of a MPS loaded with a hard PA gel and coated with Collagen I (1 or 5 mg/mL) was observed by confocal imaging. Onto the flat surface of the hydrogel, it was demonstrated that cells displayed strong tight junctions (ZO-1 staining) suggesting the formation of an intestinal epithelial barrier. Within the crypts (depth: 30 μm) the visualization of the cortical actin ring and basal nuclei supported the establishment of a polarized epithelium on the device. Moreover, at both levels, collagen staining with the fluorescent dye allowed to verify the presence of the collagen network after 21 days culture in the ‘stromal’ compartment.

### CONCLUSION

The data presented here demonstrates a simple and efficient method for the fabrication of a microphysiological system (MPS) combining the topology of a colon-like structure, with the creation of a lumen and basal compartments, and a microfluidic circuit to control the mass transport into the system.

A first proof of concept was validated using polyacrylamide hydrogels as a base material for the scaffold. In addition, the possibility to integrate additional layer of natural hydrogel materials such as collagen in order to favor the establishment of an epithelium lining, while preserving the resolution and accuracy in the human colon-like tissue architecture, was also successfully demonstrated. This method clearly opens the ways towards the creation of heterogeneous matrices reproducing different tissue architectures and gradients of stiffness found in vivo. Indeed, the design of the MPS makes possible the integration and structuration of a large variety of both synthetic and natural hydrogel materials (e.g., collagen, gelatin, laminin). The system is also compatible with current bioprinting systems to create heterogeneous models of ECM.

Important criteria for designing this MPS was the possibility to study the epithelium by several complementary means. A specific attention was paid to the ease of observation and imaging of the content and, in particular, of the epithelial tissue. Thus, glass slides were integrated on each side of the MPS. The design of the mold of imprinting crypt-like structures was also optimized for providing optimal working distances allowing wide field and confocal imaging approaches, including high-resolution analysis.

In this regard, the microfluidic coupling, allowing injection into and recovery from the system, can be of great help to perform staining on each side (luminal/apical and stromal/basal compartments) of the cultured tissue/cells. Beyond cellular staining, characterization of active or passive molecules diffusions through the hydrogel scaffold is possible. The microfluidic set-up also makes possible the real-time control and monitoring of the culture medium composition in both compartments via injection and the recovery of factors, markers, etc, secreted by the cultured cells. This later aspect is particularly interesting for the follow-up of the cell secretome and metabolism. Getting access to the different MPS compartments allows to explore interactions between the luminal content, which could include microbiota, nutrients, food additive or contaminants, and the colorectal epithelium. In order to better characterize the impact of luminal contents on the tissue, it is possible to include sensors to study parameters such as Trans Epithelial Electrical Resistances (TEER) or oxygen concentration.

Another important feature of the proposed MPS relies in the possibility to create a large epithelial surface area of approximately 1 cm² which is wider than most of the current systems described above. This notion is crucial as it may allow recovering enough material to perform either transcriptomic or proteomic approaches.

Moreover, we designed a human colon-like MPS which, being associated with colon organoids established from patients suffering of pathologies such as inflammatory bowel disease, metabolic diseases, familial adenomatous polyposis, could be used to improve the efficiency of screenings (drugs, nutrients, alimentary contaminants, microbiota studies) and tests the delivery of vectorized drugs, but also could be used for in vitro diagnostic or prognostic.

Finally, this new tool is in the line with the European policy promoting the development of strategic tools to reduce the animal experiment.

### Example 3—Diffusion of Molecules in the Porous Member

### Materials and Methods

In order to evaluate the diffusion properties of chemical species of different sizes depending on the desired polyacrylamide (PA) formulation (soft or hard stiffness), we first fabricated the chamber (2) of the microfluidic device (1) and then prepared two polyacrylamide solutions as described below:

| | |
|---|---|
| SOFT | HARD |

| | | | | |
|---|---|---|---|---|
| Acrylamide (40%) | 100 | μL | 250 | μL |
| Bis-acrylamide (2%) | 112 | μL | 112 | μL |
| APS (10%) | 10 | μL | 10 | μL |
| TEMED | 1 | μL | 1 | μL |
| H2O | 777 | μL | 627 | μL |
| Final volume | 1 | mL | 1 | mL |

500 μL of these were introduced solutions into separate MPSs and left the hydrogels to crosslink for 15 min at room temperature. After crosslinking the hydrogels, they are rinsed in a PBS solution. The luminal (first zone (4)) fluidic inlet of the microfluidic device is then connected, using fluidic tubing (Fisher Scientific, 12326899), to a syringe (BD, 307736) containing either a 3 kDa (Fisher Scientific, D3306) or 40 kDa (Fisher Scientific, D1845) FITC-dextran solution used to a concentration of 10 μM. The luminal (first zone (4)) fluid outlet of the microfluidic device is connected to a liquid waste bin. The syringe containing the FITC-dextran solution is placed on a syringe pump (KdScientific) and the dye is injected into the system at a flow rate of 10 μL/min. Monitoring of the FITC-dextran migration front over time is carried out using a wide field microscope (Apotome, Zeiss) and images are acquired at the hydrogel-liquid interface (surface (9)) of the luminal compartment (first zone (4)) of the microfluidic device every 30 seconds.

Fluorescence intensity profiles at the hydrogel-liquid interface (surface (9)) of the microfluidic device luminal compartment (first zone (4)) are extracted using Image J software. The comparison of the fluorescence intensity profiles obtained at this interface in a given time makes possible to determine a diffusion coefficient expressed in μm²/second according to Fick's law.

### Results

The results from the diffusion experiment presented above allow to validate that chemical entities can diffuse in polyacrylamide hydrogels with a stiffness between 3 and 16 kPa (i.e soft or hard PA hydrogel). The diffusion coefficients obtained are inversely proportional to the molecular weights of dextran polymers used, as expected.

This approach confirmed that the chemical entities, all sizes combined, diffuse more quickly in the soft polyacrylamide hydrogel compared to hard formulation. It is shown here that the microfluidic device (1) made it possible to experimentally determine the mechanical properties of materials (here a hydrogel) thanks to the imaging chamber modality and its ability to be coupled with a fluidic system.

### Example 4—Magnetic Peristaltism

### Materials and Methods & Results

First a chamber (2) of the microfluidic device (1) fabricated and then 500 μL of a polyacrylamide solution is poured over an elongated member and left to crosslink for 15 min at room temperature for obtaining a porous member with a microchannel. After crosslinking the hydrogel is rinsed with a PBS solution.

The following polyacrylamide solution was used:

| | HARD | |
|---|---|---|
| Acrylamide (40%) | 250 | μL |
| Bis-acrylamide (2%) | 112 | μL |
| APS (10%) | 10 | μL |
| TEMED | 1 | μL |
| H2O | 627 | μL |
| Final volume | 1 | mL |

A magnetized bead of 1 mm diameter made of steel 420c is introduced within the microchannel.

A permanent magnet (Neodymium magnet Q-40-40-20—Provider: Supermagnete) is used to exert an attractive force on a magnetized bead. The magnet is oriented at 45° above the device so that one of the vertices is closest to the ball to reach maximum gradient conditions. The distance between the magnet and the hydrogel surface may vary from 0 to 2 cm. The magnetic gradient induces at the vicinity of the device creates an attractive force on the bead that deforms the inner surface of the channel towards the magnet. This results in a compression of the hydrogel material in between the bead and the top surface.

The force is proportional to the volume of the baII, to the gradient, to the magnetization of the bead in the magnetic field B induced by the magnet. The magnetic field value changes according to the distance Dh from the magnet. As the distance decreases, the value of the field and the field gradient increase. The vertical forces generated vary from 0.1 to 30 mM over distances from 1 to 13 mm (experimental data).

Adding two magnetic beads instead of one, increases the deformation amplitude by doubling the magnetic force. In this case, the two particles aggregate spontaneously by forming doublets due to dipole-dipole interaction but not clog the channel.

The use of two balls with a magnet placed at 1 cm allows to generate a vertical force up to 2 mN corresponding to a deformation up to 15% in “hard” PAA (20 kPa).

Finally, the magnet can be moved horizontally to induce a lateral force causing a translation of the bead in the channel following the movement of the magnet. At a given z position of the magnet, the beads slide laterally in the channel while exerting a constant compression force on the material.

The lateral force varies from 0.1 to 1.5 mN.

This mechanism creates a peristaltic deformation of the porous member.

### REFERENCES

Creff J, Courson R, Mangeat T, et al. Fabrication of 3D scaffolds reproducing intestinal epithelium topography by high-resolution 3D stereolithography. Biomaterials. 2019; 221:119404. doi:10.1016/j.biomaterials.2019.119404.
Dong Y, Wang S, Ke Y, Ding L, Zeng X, Magdassi S, Long Y, 4D Printed Hydrogels: Fabrication, Materials, and Applications, Advanced Materials Technologies, 2020, 5(6)—https://doi.org/10.1002/admt.202000034
Fabre K, Berridge B, Proctor W R, et al. Introduction to a manuscript series on the characterization and use of microphysiological systems (MPS) in pharmaceutical safety and ADME applications. Lab Chip. 2020; 20(6):1049-1057. doi:10.1039/c91c01168d.
González-Díaz EC, Varghese S. Hydrogels as Extracellular Matrix Analogs. Gels. 2016; 2(3):20. Published 2016 Aug. 3. doi:10.3390/gels2030020
Helander., et al., Surface area of the digestive tract—revisited. Scand J Gastroenterol, 2014. 49(6): p. 681-689
Hennink W E, van Nostrum C F. Novel crosslinking methods to design hydrogels. Adv Drug Deliv Rev. 2002 Jan. 17; 54(1):13-36. doi: 10.1016/s0169-409-x(01)00240-x. PMID: 11755704
Joshi J, Mahajan G, Kothapalli C R. Three-dimensional collagenous niche and azacytidine selectively promote time-dependent cardiomyogenesis from human bone marrow-derived MSC spheroids. Biotechnol Bioeng. 2018; 115(8):2013-2026. doi:10.1002/bit.26714
Kim H J, Huh D, Hamilton G, Ingber D E. Human gut-on-a-chip inhabited by microbial flora that experiences intestinal peristalsis-like motions and flow. Lab Chip. 2012; 12(12):2165-2174. doi:10.1039/c21c40074j.
Kim H J, Ingber D E. Gut-on-a-Chip microenvironment induces human intestinal cells to undergo villus differentiation. Integr Biol (Camb). 2013; 5(9):1130-1140. doi:10.1039/c3ib40126j.
Nikolaev M, Mitrofanova O, Broguiere N, et al. Homeostatic mini-intestines through scaffold-guided organoid morphogenesis. Nature. 2020; 585(7826):574-578. doi:10.1038/s41586-020-2724-8.
Onfroy-Roy L, Hamel D, Foncy J, Malaquin L, Ferrand A. Extracellular Matrix Mechanical Properties and Regulation of the Intestinal Stem Cells: When Mechanics Control Fate. Cells. 2020; 9(12):2629. Published 2020 Dec. 7. doi:10.3390/cells9122629
Rosiak, J. M. and Yoshii, F. (1999) Hydrogels and Their Medical Applications. Nuclear Instrument and Methods in Physics Research Section B, 151, 56-64. https://doi.org/10.1016/SO168-583X(99)00118-4
Seed B. Silanizing glassware. Curr Protoc Mol Biol. 2001; Appendix 3:. doi:10.1002/0471142727.mba03bs28
US 2018/0200714 A1—Composite Woven Fluidic Device
Verhulsel M, Simon A, Bernheim-Dennery M, et al. Developing an advanced gut on chip model enabling the study of epithelial cell/fibroblast interactions. Lab Chip. 2021; 21(2):365-377. doi:10.1039/d01c00672f.
Wang Y, Gunasekara D B, Reed Ml, et al. A microengineered collagen scaffold for generating a polarized crypt-villus architecture of human small intestinal epithelium. Biomaterials. 2017; 128:44-55. doi:10.1016/j.biomaterials.2017.03.005
Wang Y, Kim R, Gunasekara D B, et al. Formation of Human Colonic Crypt Array by Application of Chemical Gradients Across a Shaped Epithelial Monolayer. Cell Mol Gastroenterol Hepatol. 2017; 5(2):113-130. Published 2017 Nov. 3. doi:10.1016/j.jcmgh.2017.10.007
Yu, L., et al., Microbiota dysbiosis and barrier dysfunction in inflammatory bowel disease and colorectal cancers: exploring a common ground hypothesis. J Biomed science, 2018. 25(1).

1. A microfluidic device (1) comprising a frame (32) and two opposite walls (16a,b), the two opposite walls (16a,b) and the frame (32) delimiting together a chamber (2):
the chamber (2) comprising a first zone (4) and a second zone (5),
the second zone (5) comprising a porous member (3) extending in the chamber (2) and comprising a first surface (9) and a second surface (10) opposite to the first surface (9), the first surface (9) separating the chamber (2) in the first zone (4) and in the second zone (5),
the frame (32) comprising at least a first and a second sets of ports (11, 12, 13, 14), the first set of port comprising at least two ports (11, 12) arranged in the frame (32) for fluid circulation within in the first zone (4) and the second set of ports comprising at least two ports (13, 14) arranged in the frame (32) for fluid circulation within the second zone (5), and the two ports (13, 14) of the second set of ports being open
(i) in a microchannel (15) extending through the porous member (3), or
(ii) in a cavity (19) arranged between the second surface (10) of the porous member (3) and the frame (32) of the chamber (2).

2. A microfluidic device (1) comprising a frame (32) and two opposite walls (16a,b), the two opposite walls (16a,b) and the frame (32) delimiting together a chamber (2):
the chamber (2) comprising a first zone (4) and a second zone (5),
the frame (32) comprising a movable closure (33) and at least a first and a second sets of ports (11, 12, 13, 14),
the first set of ports comprising at least two ports arranged (11, 12) in the frame (32) for fluid circulation within the first zone (4) and the second set of ports comprising at least two ports (13, 14) arranged in the frame (32) for fluid circulation within the second zone (5), and
the movable closure (33) comprising an outward face (34) and an inward face (35), the inward face (35) comprising an imprinting face (36), and the movable closure (33) extending within the chamber (2) through the first zone (4) up to an interface between the first zone (4) and the second zone (5).

3. The microfluidic device according to claim 2, wherein the second zone (5) of the chamber (2) comprises a porous member extending in the chamber (2) and comprising a first surface (9) and a second surface (10) opposite to first surface, the first surface (9) delimiting the interface between the first zone (4) and the second zone (5) of the chamber (2), and the two ports (13, 14) of the second set of ports being open
(i) in a microchannel (15) extending through the porous member (3), or
(ii) in a cavity (19) arranged between the second surface (10) of the porous member (3) and the frame (32) of the chamber (2).

4. The microfluidic device (1) according to claim 1, wherein the frame (32) comprises at least two lateral walls (6, 7) arranged between the opposite walls (16a,b).

5. The microfluidic device (1) according to claim 1, wherein the frame (32) comprises a top closure (17) and a bottom closure (8) and at least one of the top and bottom closure (17, 18) is a movable closure.

6. The microfluidic device (1) according to claim 1, wherein the frame (32) comprises a bottom closure (8), the bottom closure (8) being a fixed closure integral with the lateral walls (6, 7).

7. The microfluidic device (1) according to claim 1, wherein one or both of the opposite walls (16a,b) is or are made, in whole or in part, of a material transparent to at least one wavelength in the range from infrared to UV wavelengths.

8. The microfluidic device (1) according to claim 1, wherein
the second zone (5) comprises the porous member (3) comprising a microchannel (15a) and an additional microchannel (15b), or
the second zone (5) comprises (a) the porous member (3) comprising the microchannel (15) and (b) the cavity (19),
and the frame (32) comprises an additional set of ports comprising at least two ports (20, 21) arranged in the frame (32) for fluid circulation within the second zone (5) and being open in the additional microchannel (15a) or in the cavity (19).

9. The microfluidic device (1) according to claim 1, wherein the first surface (9) of the porous member (3) is a cell culturing surface.

10. The microfluidic device (1) according to claim 9, wherein the cell culturing surface comprises reliefs (25).

11. A microphysiological system comprising at least a microfluidic device (1) according to claim 1 and at least one cell type cultured in suitable conditions in the first and/or second zone (4, 5) of said microfluidic device (1).

12. An assembly comprising at least two microfluidic devices (1) according to claim 1, wherein the at least two microfluidic devices (1) or at least two microphysiological system being connected in series or in parallel.

13. A method for manufacturing a microfluidic device (1) according to claim 1, comprising at least the steps of:
providing a chamber (2) delimited by a frame (32) and two opposite walls (16a,b),
forming a porous member (3) in the chamber (2), the porous member (3) comprising a first surface (9) and a second surface (10), the first surface (9) separating the chamber (2) in a first and a second zones (4,5), wherein
the frame (32) comprises at least a first and a second sets of ports (11, 12, 13, 14), the first set of ports (11, 12) comprising at least two ports arranged in the frame (32) for fluid circulation within the first zone (4) and the second set of ports comprising at least two ports (13, 14) arranged in the frame (32) for fluid circulation within the second zone (5), and the two ports (13, 14) of the second set of ports being open
(i) a microchannel (15) extending through the porous member (3), or
(ii) in a cavity (19) arranged between the second surface (10) of the porous member (3) and the frame (32) of the chamber (2).

14. The manufacturing method according to claim 13, wherein the microchannel (15) in the porous member (3) is obtained by:
positioning within the chamber (2) at least one elongated member extending through the ports of the second set of ports (13, 14), and
casting in the chamber (2) at least one material suitable to embed said elongated member and to form a porous member (3) and a microchannel (15) extending through said first porous member (3).

15. The manufacturing method according to claim 13, further comprising a step of forming reliefs (25) on the first surface (9) of the porous member (3) by contacting the first surface (9) with a movable closure (33) comprising an outward face (34) and an inward face (35), the inward face comprising an imprinting face (36) for imprinting reliefs (25) on the surface (9), and the movable closure (33) being arranged to extend within the chamber (2) through the first zone (4) up to the interface with the second zone (5) to contact with the first surface (9) of the porous member (3).

16. (canceled)

17. (canceled)

18. Method for culturing isolated cells, modelling a cell tissue, or modelling a biological surface comprising at least the steps of:
providing at least one microfluidic device (1) according to claim 1,
culturing, in suitable conditions, at least one cell type in at least one of at least first and second zones (4,5), the first and/or second zones (4,5) containing a cell culture medium in circulation through at least a set of at least two ports (11, 12, 13, 14).

19. The method according to claim 18, wherein the cells are cultured on the first surface (9) of the porous member (3).

20. The microfluidic device (1) according to claim 2, wherein the frame (32) comprises at least two lateral walls (6, 7) arranged between the opposite walls (16a,b).

21. The microfluidic device (1) according to claim 20, wherein the frame (32) comprises a top closure (17) and a bottom closure (8) and at least one of the top and bottom closure (17, 18) is a movable closure.

22. The microfluidic device (1) according to claim 2, wherein the frame (32) comprises a bottom closure (8), the bottom closure (8) being a fixed closure integral with the lateral walls (6, 7).

23. The microfluidic device (1) according to claim 2, wherein one or both of the opposite walls (16a,b) is or are made, in whole or in part, of a material transparent to at least one wavelength in the range from infrared to UV wavelengths.

24. The microfluidic device (1) according to claim 3, wherein
the second zone (5) comprises the porous member (3) comprising a microchannel (15a) and an additional microchannel (15b), or
the second zone (5) comprises (a) the porous member (3) comprising the microchannel (15) and (b) the cavity (19),
and the frame (32) comprises an additional set of ports comprising at least two ports (20, 21) arranged in the frame (32) for fluid circulation within the second zone (5) and being open in the additional microchannel (15a) or in the cavity (19).

25. The microfluidic device (1) according to claim 3, wherein the first surface (9) of the porous member (3) is a cell culturing surface.

26. An assembly comprising at least two microphysiological system according to claim 11, wherein the at least two microfluidic devices (1) or at least two microphysiological system being connected in series or in parallel.

27. (canceled)

28. (canceled)
